(19) United States
(12) Patent Application Publication
(10) Pub. No.: US 2024/0228553 A1
(43) Pub. Date: Jul. 11, 2024

(54) FUSION PROTEINS COMPRISING GG REPEAT SEQUENCES

(71) Applicant: Numaferm GmbH, Düsseldorf (DE)

(72) Inventors: Christian Schwarz, Düsseldorf (DE); Janpeter Stock, Düsseldorf (DE); Jose Montoya, Düsseldorf (DE)

(21) Appl. No.: 18/550,967

(22) PCT Filed: Sep. 21, 2021
(86) PCT No.: PCT/EP2021/075891

§ 371 Date: Sep. 17, 2023

(30) Foreign Application Priority Data
Mar. 18, 2021 (WO) PCT/EP2021/056947

(51) Int. Cl. C07K 14/245; C12N 15/62

(52) U.S. Cl. CPC C07K 14/245; C12N 15/62; C07K 2319/00

(57) ABSTRACT

The present invention relates to polypeptides comprising a first amino acid sequence comprising one or more GG repeat sequences and a peptide or polypeptide of interest in form of a fusion protein that exhibits increased renaturation efficiency and optionally also improved expression. Also encompassed are nucleic acids encoding these polypeptides, host cells that comprise said nucleic acids, and methods for protein expression and renaturation using said nucleic acids, host cells and polypeptides.

### CROSS REFERENCE TO RELATED APPLICATION

This application is a National Stage of International Application No. PCT/EP2021/075891, filed Sep. 21, 2021; which claims the benefit of International Application No. PCT/EP2021/056947, filed Mar. 18, 2021, under 35 U.S.C. 119(a); the disclosure of each of which is incorporated herein by reference in its entirety.

### REFERENCE TO A SEQUENCE LISTING

The present specification is being filed with a Sequence Listing in Computer Readable Form (CRF), which is entitled SEQ_LISTING_ST25.txt of 270,480 bytes in size; the content of which is incorporated herein by reference in its entirety.

### FIELD OF THE INVENTION

The present invention lies in the field of molecular biology, recombinant peptide and protein expression and relates to amino acid sequences that support the refolding and renaturation of a peptide or protein of interest recombinantly expressed as a fusion protein.

### BACKGROUND OF THE INVENTION

To date, recombinant protein/enzyme production for use in industrial processes is widely established. It is expected that in the future more and more industrial processes that are currently based on traditional chemistry will be adapted to involve recombinant technologies.

The type 1 secretion system (T1SS) is a relatively simple yet highly conserved secretion strategy used throughout Gram-negative bacteria to translocate small substrates as well as extremely large proteins to the extracellular environment. Much of our current knowledge of T1SS has been gathered over the last several decades from studies of the T1SS toxins HlyA and CyaA of Escherichia coli and Bordetella pertussis, respectively. These toxins, and the majority of type 1 secreted proteins, belong to the repeats-in-toxin (RTX) family (Welch (1991), Mol Microbiol 5:512-528).

While the technologies based on the T1SS, in particular fusion proteins of peptides and proteins to be expressed with HlyA are known in the art, for example from the international patent publications WO 2013/057312 A1 and WO 2014/170430 A1, and are commercially available, it has been found that in various instances high yields are prevented by suboptimal solubility or susceptibility to proteolysis of the fusion constructs. Thus, there still exists need for further optimized methods that allow more efficient production of peptides and proteins that overcome some of the drawbacks of existing methods.

### SUMMARY OF THE INVENTION

The present invention is based on the inventor's surprising finding that certain amino acid sequences derived from RTX proteins as defined herein when fused to a peptide or protein of interest that is to be recombinantly produced by expression of a fusion protein in a host cell provide for increased renaturation efficiency, while at the same time providing for good stability and/or solubility as well as similar or even increased expression rates relative to longer sequences and thus allow higher yields of the desired (fused) product. Specifically, this means that it allows for the production of high titer inclusion bodies with high initial purities after extraction while at the same time protecting fused peptides/proteins from degradation, e.g. proteolysis, desamidation, truncation, oxidation and the like and allowing their renaturation from a denatured into a properly folded functional state by means of the RTX-derived fusion tag.

In a first aspect, the present invention is therefore directed to an isolated polypeptide comprising a first and a second amino acid sequence, wherein the first amino acid sequence is 30 to 200 amino acids in length, and comprises at least one GG repeat sequences of the general consensus sequence GGxGxDxUx, wherein x can be any amino acid and U is a hydrophobic, large amino acid selected from F, I, L, M, W and Y, and wherein the second amino acid sequence is the at least one peptide or polypeptide of interest, and wherein the first and second amino acid sequence are heterologous to each other.

In various embodiments, the first amino acid sequence comprises at least 2, at least 3, at least 4, at least 5 or at least 6 GG repeat sequence of the general consensus sequence GGxGxDxUx. It may be preferred, in various embodiments, that the first amino acid sequence comprises at least 2 or 3 GG repeat sequences. It may also be preferred that if the first amino acid sequence comprises two or more GG repeat sequences, at least two thereof are directly adjacent to each other, i.e. the C-terminal end of the first one is linked to the N-terminal end of the second one, etc. In various embodiments, the number of GG repeats in the first amino acid sequence may also be 4, 5, 6 or even more. It has been found, for example, that in some embodiments constructs where the first amino acid sequence comprises 6 GG repeat sequences perform particularly well.

In various embodiments, the first amino acid sequence comprises the general structure, from N- to C-terminus,
(i) GGR1-Linker-GGR2, or
(ii) GGR1-Linker-GGR2-Linker-GGR3; or
(iii) GGR1-Linker-GGR2-Linker-GGR3-Linker-GGR4; or
(iv) GGR1-Linker-GGR2-Linker-GGR3-Linker-GGR4-Linker-GGR5; or
(v) GGR1-Linker-GGR2-Linker-GGR3-Linker-GGR4-Linker-GGR5-Linker-GGR6
(vi) GGR1-Linker-GGR2-Linker-GGR3-Linker-GGR4-Linker-GGR5-Linker-GGR6-Linker-GGR7
(vii) GGR1-Linker-GGR2-Linker-GGR3-Linker-GGR4-Linker-GGR5-Linker-GGR6-Linker-GGR7-Linker-GGR8
   wherein GGR1, GGR2, GGR3, GGR4, GGR5, GGR6, GGR7 and GGR8 are GG repeats of the consensus sequence GGxGxDxUx, wherein x can be any amino acid and U is a hydrophobic, large amino acid selected from F, W, Y, I, L and M, and wherein each “Linker” is independently either a peptide bond or an amino acid sequence of 1 to 25 amino acids, preferably 1 to 20 or 1 to 15 amino acids in length. Said general structure given above as (i)-(v) may be flanked by additional N- and/or C-terminal amino acid sequences that may be 1 to 100 amino acids in length, preferably 1 to 80, 1 to 70, 1 to 60, 1 to 50, 1 to 40, 1 to 30, 1 to 20, 1 to 10 or 1 to 5 amino acids in length.

In various embodiments, the U in the consensus sequence is selected from F, I, L, M and Y.

In various embodiments, the consensus sequence is GGX¹GX²DX³UX⁴, wherein X¹, X², X³ and X⁴ may be any amino acid with the exception of P. In various embodiments, X¹ is selected from G, A, L, V, I, M, F, S, T, Q; Y, K, R, D, E, N, and Y, for example from G, A, E, S, T, Q, L, R and D, or from G, A, E, S and T; and/or X² is selected from N, D, A, G, S, H, T, E, M, R and H, for example from N, D, A, S, and H, or from N, D, A, and S; and/or X³ is selected from A, K, V, L, I, F, M, R, Y, S, L, T, V, Q, N, D, E, and H, for example from T, R, V, L, S, I, A, Y, Q, D and H, or from T, R, V, L, S and I, or from T, R and V; and/or X⁴ is selected from W, K, R, Y, V, L, T, H, D, A, M, E, F, I, S, N and Q, for example from V, L, I, F, S, R, N, Y and T.

In various embodiments, the at least one GG repeat sequence is selected from any one of GGKGNDKLY (SEQ ID NO:1), GGEGDDLLK (SEQ ID NO:2), GGYGNDIYR (SEQ ID NO:3), GGTGNDRLW (SEQ ID NO:4), GGTGADIFV (SEQ ID NO:5), GGGGDDIIV (SEQ ID NO:6), GGKGDDYLE (SEQ ID NO:7), GGAGNDSYF (SEQ ID NO:8), GGSGNDLLI (SEQ ID NO:9), GGAGNDIIY (SEQ ID NO:10), GGGGGDTLW (SEQ ID NO:11), GGAGADTFV (SEQ ID NO:12), GGQGNDVFV (SEQ ID NO:13), GGAGNDLME (SEQ ID NO:14), GGLGDDHLV (SEQ ID NO:15), GGRGSDLLI (SEQ ID NO:16), GGDGADRIS (SEQ ID NO:17), GGAGNDIIR (SEQ ID NO:18), GGGGHDRMQ (SEQ ID NO:19), GGAGDDTFV (SEQ ID NO:20), GGNGDDQLY (SEQ ID NO:21), GGDGNDKLI (SEQ ID NO:22), GGAGNDYLN (SEQ ID NO:23), GGDGDDELQ (SEQ ID NO:24), and GGAGADVLS (SEQ ID NO:63). If two or more GG repeat sequences are comprised in the first amino acid sequence, each may be selected independently from those listed above.

In various embodiments, the at least one GG repeat sequence is selected from any one of GGAGNDSYF (SEQ ID NO:8), GGAGNDIIY (SEQ ID NO:10), GGAGADTFV (SEQ ID NO:12), GGAGNDLME (SEQ ID NO:14), GGAGNDIIR (SEQ ID NO:18), GGAGDDTFV (SEQ ID NO:20), GGAGNDYLN (SEQ ID NO:23), GGAGADVLS (SEQ ID NO:63). GGAGSDYLS (SEQ ID NO:165), GGAGADQLF (SEQ ID NO:166), GGAGDDTTV (SEQ ID NO:167), GGAGADDLT (SEQ ID NO:168), GGAGADNFI (SEQ ID NO:169), GGAGNDEVH (SEQ ID NO:170), GGAGNDYLS (SEQ ID NO:171), GGAGNDSLF (SEQ ID NO:172), GGAGFDILI (SEQ ID NO:173), GGAGNDVAF (SEQ ID NO:174), GGAGNNDIYH (SEQ ID NO:175), GGAGADSFV (SEQ ID NO:176), GGAGADALY (SEQ ID NO:177), GGAGSDAIV (SEQ ID NO:178), GGAGEDTFR (SEQ ID NO:179), GGAGHDRLA (SEQ ID NO:180), GGAGADTFV (SEQ ID NO:181), GGAGDDQLS (SEQ ID NO:182), GGAGDDVLE (SEQ ID NO:183), GGAGTDHLD (SEQ ID NO:184), GGAGNDRID (SEQ ID NO:185), GGAGADQLW (SEQ ID NO:186), GGAGNDTFV (SEQ ID NO:187), GGAGGDLLD (SEQ ID NO:188), GGAGEDSFR (SEQ ID NO:189), GGAGNDLME (SEQ ID NO:190), GGAGMDALH (SEQ ID NO:191), GGAGTDTLV (SEQ ID NO:192), GGAGADTLY (SEQ ID NO:193), GGAGADELT (SEQ ID NO:194), GGDGADRIS (SEQ ID NO:17), GGAGADRLD (SEQ ID NO:368), GGDGNDKLI (SEQ ID NO:22), GGDGDDELQ (SEQ ID NO:24), GGDGNDVLL (SEQ ID NO:195), GGDGNDSLV (SEQ ID NO:367), GGDGADLLF (SEQ ID NO:196), GGDGTDFLL (SEQ ID NO:197), GGEGDDLLK (SEQ ID NO:2), GGEGHDFVS (SEQ ID NO:198), GGEGDDRVY (SEQ ID NO:199), GGEGADLLF (SEQ ID NO:200), GGEGRDSLY (SEQ ID NO:227), GGEGNDHLR (SEQ ID NO:201), GGEGADRLI (SEQ ID NO:202), GGFGNDEVN (SEQ ID NO:203), GGGGDDIIV (SEQ ID NO:6), GGGGGDTLW (SEQ ID NO:11), GGGGHDRMQ (SEQ ID NO:19), GGGGSDIMR (SEQ ID NO:204), GGGGNDILI (SEQ ID NO:205), GGGGNDRLE (SEQ ID NO:206), GGGGSDMFV (SEQ ID NO:207), GGKGNDKLY (SEQ ID NO:1), GGKGDDYLE (SEQ ID NO:7), GGLGDDHLV (SEQ ID NO:15), GGLGSDVLD (SEQ ID NO:208), GGLGSDQLF (SEQ ID NO:209), GGLGADTLI (SEQ ID NO:210), GGLGSDAFA (SEQ ID NO:358), GGMGADELT (SEQ ID NO:211), GGNGDDQLY (SEQ ID NO:21), GGNGVDLAN (SEQ ID NO:369), GGQGNDVFV (SEQ ID NO:13), GGQGRDQLH (SEQ ID NO:212), GGRGSDLLI (SEQ ID NO:16), GGRGSDIFA (SEQ ID NO:213), GGRGSDLLD (SEQ ID NO:214), GGSGNDLLI (SEQ ID NO:9), GGSGNDRLI (SEQ ID NO:215), GGSGNDRLD (SEQ ID NO:216), GGSGNDDLS (SEQ ID NO:217), GGSGDDRYQ (SEQ ID NO:218), GGSGSDTFV (SEQ ID NO:219), GGTGNDRLW (SEQ ID NO:4), GGTGADIFV (SEQ ID NO:5), GGTGNDLVS (SEQ ID NO:220), GGTGGDTLS (SEQ ID NO:221), GGTGHDTLI (SEQ ID NO:222), GGTGSDRLV (SEQ ID NO:223), GGTGNDTYI (SEQ ID NO:224), GGTGRDVFL (SEQ ID NO:225), GGVGADTMT (SEQ ID NO:226), and GGYGNDIYR (SEQ ID NO:3). If two or more GG repeat sequences are comprised in the first amino acid sequence, each may be selected independently from those listed above.

In various embodiments, the at least one GG repeat sequence is selected from any one of GGAGNDIIR (SEQ ID NO:18), GGAGDDTFV (SEQ ID NO:20), GGAGSDYLS (SEQ ID NO:165), GGAGADQLF (SEQ ID NO:166), GGAGADDLT (SEQ ID NO:168), GGAGADNFI (SEQ ID NO:169), GGAGNDEVH (SEQ ID NO:170), GGAGNDYLS (SEQ ID NO:171), GGDGADRIS (SEQ ID NO:17), GGDGNDVLL (SEQ ID NO:195), GGDGADLLF (SEQ ID NO:196), GGEGHDFVS (SEQ ID NO:198), GGEGDDRVY (SEQ ID NO:199), GGEGADLLF (SEQ ID NO:200), GGEGRDSLY (SEQ ID NO:227), GGFGNDEVN (SEQ ID NO:203), GGGGHDRMQ (SEQ ID NO:19), GGLGDDHLV (SEQ ID NO:15), GGLGSDAFA (SEQ ID NO:358), GGLGSDVLD (SEQ ID NO:208), GGLGSDQLF (SEQ ID NO:209), GGRGSDLLI (SEQ ID NO:16), GGRGSDIFA (SEQ ID NO:213), GGTGNDLVS (SEQ ID NO:220), GGTGGDTLS (SEQ ID NO:221), and GGVGADTMT (SEQ ID NO:226). If two or more GG repeat sequences are comprised in the first amino acid sequence, each may be selected independently from those listed above.

In various embodiments, the isolated polypeptide comprises the general structure of any one of (i)-(v) above and:
a) GGR1 is GGKGNDKLY (SEQ ID NO:1) and/or GGR2 is GGEGDDLLK (SEQ ID NO:2) and/or GGR3 is GGYGNDIYR (SEQ ID NO:3);
b) GGR1 is GGTGNDRLW (SEQ ID NO:4) and/or GGR2 is GGAGADVLS (SEQ ID NO:63) and/or GGR3 is GGTGADIFV (SEQ ID NO:5);
c) GGR1 is GGGGDDIIV (SEQ ID NO:6) and/or GGR2 is GGKGDDYLE (SEQ ID NO:7) and/or GGR3 is GGAGNDSYF (SEQ ID NO:8);
d) GGR1 is GGSGNDLLI (SEQ ID NO:9) and/or GGR2 is GGAGNDIIY (SEQ ID NO:10) and/or GGR3 is GGGGGDTLW (SEQ ID NO:11) and/or GGR4 is GGAGADTFV (SEQ ID NO:12);
e) GGR1 is GGQGNDVFV (SEQ ID NO:13) and/or GGR2 is GGAGNDLME (SEQ ID NO:14); or
f) GGR1 is GGLGDDHLV (SEQ ID NO:15) and/or GGR2 is GGRGSDLLI (SEQ ID NO: 16) and/or GGR3 is GGDGADRIS (SEQ ID NO:17) and/or GGR4 is GGAGNDIIR (SEQ ID NO:18) and/or GGR5 is GGGGHDRMQ (SEQ ID NO:19) and/or GGR6 is GGAGDDTFV (SEQ ID NO:20); or
g) GGR1 is SEQ ID NO:21 and/or GGR2 is SEQ ID NO:22 and/or GGR3 is SEQ ID NO:24;
h) GGR1 is GGKGNDKLY (SEQ ID NO:1) and GGR2 is GGEGDDLLK (SEQ ID NO:2) and GGR3 is GGYGNDIYR (SEQ ID NO:3);
i) GGR1 is GGTGNDRLW (SEQ ID NO:4) and GGR2 is GGAGADVLS (SEQ ID NO:63) and GGR3 is GGTGADIFV (SEQ ID NO:5);
j) GGR1 is GGGGDDIIV (SEQ ID NO:6) and GGR2 is GGKGDDYLE (SEQ ID NO:7) and GGR3 is GGAGNDSYF (SEQ ID NO:8);
k) GGR1 is GGSGNDLLI (SEQ ID NO:9) and GGR2 is GGAGNDIIY (SEQ ID NO:10) and GGR3 is GGGGGDTLW (SEQ ID NO:11) and GGR4 is GGAGADTFV (SEQ ID NO:12);
l) GGR1 is GGQGNDVFV (SEQ ID NO:13) and GGR2 is GGAGNDLME (SEQ ID NO:14); or
m) GGR1 is GGLGDDHLV (SEQ ID NO:15) and GGR2 is GGRGSDLLI (SEQ ID NO:16) and GGR3 is GGDGADRIS (SEQ ID NO:17) and GGR4 is GGAGNDIIR (SEQ ID NO:18) and GGR5 is GGGGHDRMQ (SEQ ID NO: 19) and GGR6 is GGAGDDTFV (SEQ ID NO:20); or
n) GGR1 is SEQ ID NO:21 and GGR2 is SEQ ID NO:22 and GGR3 is SEQ ID NO:24.
o) GGR1 is SEQ ID NO:221 and/or GGR2 is SEQ ID NO: 195 and/or GGR3 is SEQ ID NO:168 and/or GGR4 is SEQ ID NO:226 and/or GGR5 is SEQ ID NO: 169; or
p) GGR1 is SEQ ID NO:170 and/or GGR2 is SEQ ID NO:227 and/or GGR3 is SEQ ID NO:208 and/or GGR4 is SEQ ID NO:171 and/or GGR5 is SEQ ID NO:209 and/or GGR6 is SEQ ID NO: 196 and/or GGR7 is SEQ ID NO:213; or
q) GGR1 is SEQ ID NO: 198 and/or GGR2 is SEQ ID NO:203 and/or GGR3 is SEQ ID NO: 199 and/or GGR4 is SEQ ID NO:220 and/or GGR5 is SEQ ID NO: 165 and/or GGR6 is SEQ ID NO: 166 and/or GGR7 is SEQ ID NO:200 and/or GGR8 is SEQ ID NO: 358.

In various embodiments of a) to q), the isolated polypeptide may lack any one or more of GGR1 to GGR8 as long as at least 1, preferably at least 2 or at least 3 GG repeat sequences remain.

In various embodiments, the first amino acid sequence of the isolated polypeptide comprises at least two GG repeats and the at least two GG repeats are directly linked by a peptide bond. The isolated polypeptide may comprise at least three GG repeats and the at least three GG repeats may be directly linked to each other by a peptide bond. In such embodiments, there is no linker sequence present between the GG repeat sequence motifs, i.e. the “Linker” in general structures (i) to (vii) is a bond. In such embodiments, the first amino acid sequence can comprise additional GG repeats that are connected to the directly linked GG repeats by a “Linker” that is not a peptide bond. It may be preferred, in some embodiments, that the isolated polypeptide does not comprise GG repeat sequences that are not contained in the first and, optionally, the second amino acid sequence, for example does not comprise GG repeat sequences that are not contained in the first amino acid sequence.

In various embodiments, the first amino acid sequence is 30 to 150 amino acids, preferably 30 to 120 or 30 to 100 amino acids in length.

In various embodiments, the first amino acid sequence does not comprise a secretion signal. This may mean that the first amino acid sequence does not comprise the C-terminal sequence TTSA (SEQ ID NO:25).

In various embodiments, the first amino acid sequence may be a fragment of a naturally occurring RTX protein, such as those disclosed herein below. RTX (repeats-in-toxin) proteins are exoproteins of Gram-negative bacteria with diverse biological functions. Their common feature is the unique mode of export across the bacterial envelope via the type I secretion system (T1SS) and the presence of one or more GG repeat motifs. Such fragments are typically at least 30 amino acids in length and comprise at least one GG repeat sequence, preferably two or three GG repeat sequences, as defined herein. In various embodiments, said fragments do not retain the functional C-terminal secretion signal, i.e. at least the C-terminal 40, preferably 50 or 60 amino acids are not part of the fragment. The fragments are thus typically N-terminal fragments that additionally lack most of the N-terminus and retain only the amino acid sequences in direct vicinity of the retained GG repeats.

These RTX (repeats-in-toxin) proteins may be allocrits of a T1SS (type I secretion system) and may be selected from the group consisting of, without limitation, HlyA, CyaA, EhxA, LktA, PILktA, PasA, PvxA, MmxA, LtxA, ApxIA, ApxIIA, ApxIIIA, ApxIVA, ApxI, Apx11, AqxA, VcRtxA, VvRtxA, MbxA, RTX cytotoxin, RtxL1, RtxL2, FrhA, LipA, TliA, PrtA, PrtSM, PrtG, PrtB, PrtC, AprA, AprX, ZapA, ZapE, Sap, HasA, colicin V, LapA, ORF, RzcA, RtxA, XF2407, XF2759, RzcA, RsaA, Crs, CsxA, CsxB, SlaA, SwmA, S111951, NodO, PlyA, PlyB, FrpA, FrpC,
RTX1 (SCP-domain containing protein, Aromatoleum aromaticum strain EbN1; NCBI Reference WP_011239843.1; UniProt Accession No. Q5NXL6),
RTX3 (Serralysin (Caulobacter sp. strain K31); UniProt Accession No. BOT558),
RTX7 (uncharacterized protein of Pelobacter propionicus; UniProt Accession No. A1ARK7; NCBI Reference WP_011736233.1),
RTX8 (Metallopeptidase AprX (Pseudomonas fluorescens strain ATCC BAA-477/NRRL B-23932/Pf-5); UniProt Accession No. Q4KDU3; NCBI Reference WP_011060780.1),
RTX9 (glycosyl hydrolase family 16, hemolysin-type calcium-binding repeat protein (Rhodobacter sphaeroides strain ATCC 17023/DSM 158/JCM 6121); UniProt Accession No. Q3J0J9; NCBI Reference WP_011338282.1),
RTX12 (endonuclease/exonuclease/phosphatase (Caulobacter sp. strain K31); UniProt Accession No. B0T829; NCBI Reference WP_012287684.1),
RTX14 (Glycoside hydrolase family 16 (Caulobacter sp. strain K31); UniProt Accession No. B0T438),
RTX16 (lipase class 3 (Chlorobium phaeobacteroides strain DSM266); UniProt Accession No. A1BIU7; NCBI Reference WP_011746110.1),
RTX18 (animal haem peroxidase (Leptotrix cholodnii srain ATCC 51168/LMG 8142/SP-6); UniProt Accession No. B1Y442; NCBI Reference WP_012347322.1),
RTX19 (putative outer membrane adhesin like protein (Magnetococcus marinus strain ATCC BAA-1437/JCM 17883/MC-1); UniProt Accession No. A0L6L9; ENA Reference ABK43612.1),
RTX20 (putative outer membrane adhesin like protein (Magnetococcus marinus strain ATCC BAA-1437/JCM 17883/MC-1); UniProt Accession No. A0LDY6; NCBI Reference WP_011715232.1),
RTX21 (Glycerophosphoryl diester phosphodiesterase (Methylobacterium nodulans (strain LMG 21967/CNCM I-2342/ORS 2060); UniProt Accession No. B8ICD8; EMBL Reference ACL55526.1),
RTX22 (5′ nucleotidase domain protein (Methylobacterium sp. strain 4-64); UniProt Accession No. B0UQ63; NCBI Reference WP_012331605.1),
RTX24 (polyurethanase A (Pseudomonas fluorescens strain ATCC BAA-477/NRRL B 23932/Pf-5); UniProt Accession No. Q4KBS6; NCBI Reference WP_011061486.1),
RTX26 (extracellular alkaline metalloprotease AprA (Pseudomonas fluorescens strain ATCC BAA-477/NRRL B-23932/Pf-5); UniProt Accession No. Q4KBR8; NCBI Reference WP_011061494.1),
RTX28 (hemolysin type calcium-binding protein (Rhodobacter sphaeroides strain ATCC 17023/DSM 158/JCM 6121); UniProt Accession No. Q3IVG4; NCBI Reference WP_011331290.1),
RTX29 (neutral zinc metallopeptidase (Rhodobacter sphaeroides strain ATCC 17023/DSM 158/JCM 6121); UniProt Accession No. Q3J1D3; NCBI Reference WP_011338088.1),
RTX30 (glycoside hydrolase family 16 (Rhodobacter sphaeroides strain ATCC 17029/ATH 2.4.9); UniProt Accession No. A3PLQ2; NCBI Reference WP_011841485.1),
RTX31 (peptidase domain protein (Sinorhizobium medicae strain WSM419); UniProt Accession No. A6UK74; NCBI Reference WP_011970163.1),
RTX32 (Serralysin (Sinorhizobium medicae strain WSM419); UniProt Accession No. A6UK83; NCBI Reference WP_011970172.1), and
RTX33 (hemolysin type calcium-binding region (Verminephrobacter eiseniae strain EF01-2); UniProt Accession No. A1WL15; NCBI Reference WP_011810323.1).

As disclosed herein, these proteins are not used as such as the first amino acid sequence, as they are typically more than 200 amino acids in length, but rather fragments thereof that comprise 1 or more GG repeat sequences and, preferably, do not comprise the (C-terminal) secretion signal are used.

In various embodiments, such fragments may be derived from a known C-terminal fragment of HlyA, namely HlyA1 (SEQ ID NO:26). Said known fragment comprises 3 GG repeats having the amino acid sequences set forth in SEQ ID Nos. 1-3 in positions 11-19, 29-37, and 38-46 using the positional numbering of SEQ ID NO:26. These GG repeats, namely 1, 2 or all 3, are preferably retained in the fragments thereof that are used as the first amino acid sequence in accordance with the present invention. In such embodiments, the first amino acid sequence (and also the total isolated polypeptide) comprises less than 218 continuous amino acids of the amino acid sequence set forth in SEQ ID NO:26. In such embodiments, the first amino acid sequence is derived from the amino acid sequence set forth in SEQ ID NO:26 by any one or more of an N-terminal truncation, a C-terminal truncation or a deletion of one or more amino acids, in particular a C-terminal truncation. In such embodiments, the first amino acid sequence may be at least 50 amino acids in length, preferably at least 60, 70, 80, 90, 100, 110, 120, 130, 140, 150, 160, 170, 180, 190, 200, 210, 211, 212, 213, 214, 215, 216, or 217 amino acids long. In various embodiments, the fragment of SEQ ID NO:26 has a C-terminal truncation, for example up to 30, up to 35, up to 40, up to 45, up to 50, up to 55 or up to 60 amino acids, or may lack the amino acids corresponding to amino acids 135-218, 130-218, 125-218, 120-218, 115-218, 110-218, 105-218, 100-218, 95-218, 90-218, 85-218, 80-218, 75-218, 70-218, 65-218, 60-218, 55-218, 50-218, 45-218 or 40-218 of SEQ ID NO:26, the amino acid sequence of such fragments being set forth in SEQ ID Nos. 34-53, respectively. Also encompassed are all other deletions up to residue 40, preferably up to residue 46, using the positional numbering of SEQ ID NO:26. In various embodiments, any one or more of the amino acids up to the position corresponding to position 109 of SEQ ID NO:26 (starting from 218 and counting backwards) may be deleted. Such deletions may cover the amino acid residues at the positions corresponding to positions 185-218, positions 165-218, positions 135-218 or positions 110-218 of SEQ ID NO:26. Also encompassed are any deletions of ranges between those recited above, such as 184-218, 183-218, 182-218, 181-218, 180-218, 179-218, 178-218, 177-218, 176-218, 175-218, 174-218, 173-218, 172-218, 171-218, 170-218, 169-218, 168-218, 167-218, 166-218, 164-218, 163-218, 162-218, 161-218, 160-218, 159-218, 158-218, 157-218, 156-218, 155-218, 154-218; 153-218; 152-218; 151-218; 150-218; 149-218; 148-218; 147-218; 146-218; 145-218; 144-218; 143-218; 142-218; 141-218; 140-218; 139-218; 138-218; 137-218; 136-218; 134-218; 133-218; 132-218; 131-218; 130-218; 129-218; 128-218; 127-218; 126-218; 125-218; 124-218; 123-218; 122-218; 121-218; 120-218; 119-218; 118-218; 117-218; 116-218; 115-218; 114-218; 113-218; 112-218; and 111-218. Furthermore, deletions of the amino acids at the positions corresponding to positions 215-218 of SEQ ID NO:26 may be combined with deletions at the positions corresponding to positions 165-182, such as 165-168, 165-169, 165-170, 165-171, 165-172, 165-173, 165-174, 165-175, 165-176, 165-177, 165-178, 165-179, 165-180, and 165-181. In various embodiments, the fragments of the amino acid sequence set forth in SEQ ID NO:26 include at least the amino acids corresponding to positions 11-19, 29-37, and/or 38-46 of SEQ ID NO: 1, preferably 2 or all three of these amino acid stretches, since these represent the GG repeat sequences. In various embodiments, the fragments of SEQ ID NO:26 thus comprise at least amino acids 10-46, 10-50, 10-55, 1-46, 1-50, 1-55, 1-60, 1-65, or 1-68, for example 1-75, 1-90, 1-100, 1-105 or 1-109 of SEQ ID NO:26. In various embodiments, the first amino acid sequence is thus a fragment of SEQ ID NO:26 that has a C-terminal truncation, for example of amino acids in the positions corresponding to positions 215-218, 214-218, 185-218, 165-218, 135-218, or 111-218 of SEQ ID NO:26. In various embodiments the C-terminal end up to amino acid 110 (not including 110) in the numbering of SEQ ID NO:26 may be truncated. In various other embodiments, the amino acids in positions corresponding to positions 215-218 or 214-218 of SEQ ID NO:26 and additionally in the positions corresponding to positions 164-168, 165-168, 164-173, 165-173, 164-183, 165-183 or 165-182 of SEQ ID NO:26 may be deleted. In various embodiments any continuous 4 or more amino acids in the region corresponding to positions 164-183 of SEQ ID NO:26, in particular starting from position 164 or 165 and up to position 182 or 183 may be deleted. Exemplary embodiments of such truncations are set forth in SEQ ID Nos. 27-33. Further embodiments of such fragments of HlyA1 are set forth in SEQ ID Nos. 54-56. In various embodiments, such fragments of HlyA1 may, N-terminally to the sequences recited above, comprise any one of the amino acid sequences set forth in SEQ ID Nos. 21-24 and 102-105. These represent the GG repeat sequences or GG repeat-containing sequences of HlyA that are not included in the known fragment HlyA1 but are located N-terminal to the HlyA1 fragment in the native HlyA sequence. Exemplary fragments of HlyA that may be used as first amino acid sequences of the present invention are set forth in SEQ ID Nos. 34-56 and 117-155.

Examples of other fragments of RTX proteins are set forth in SEQ ID Nos. 57-62 and 65-105. Fragments of RTX1 are set forth in SEQ ID Nos. 57-62 and 364. These comprise the GG repeat sequences set forth in SEQ ID Nos. 4, 5 and 63. Fragments of RTX7 are set forth in SEQ ID Nos. 65-69, 371. These comprise GG repeat sequences set forth in SEQ ID Nos. 6-8. Fragments of RTX8 are set forth in SEQ ID Nos. 70-76. These comprise GG repeat sequences set forth in SEQ ID Nos. 9-12. Fragments of RTX24 are set forth in SEQ ID Nos. 77-80. These comprise GG repeat sequences set forth in SEQ ID Nos. 13-14. Fragments of RTX12 are set forth in SEQ ID Nos. 81-101. These comprise GG repeat sequences set forth in SEQ ID Nos. 15-20. The GG repeat core sequences of RTX1, RTX9, RTX12, RTX18, RTX21, RTX22, RTX30, RTX33, RTX28, RTX24, RTX19, RTX16, RTX26, RTX3, RTX20, RTX31, RTX14, RTX32 and RTX29 are set forth in SEQ ID Nos. 235, 241, 246, 251, 252, 256, 261, 269, 274, 278, 282, 289, 291, 293, 295, 297, 298, 300, 359, 360 and 364. In various embodiments, the first amino acid sequence comprises, consists essentially of or consists of any of these sequences, in particular those derived from RTX1, RTX12, RTX16, RTX18, RTX22, RTX21, RTX26, RTX30 and RTX33, for example RTX1, RTX12, RTX16 and RTX18. Further suitable first amino acid sequences derived from RTX proteins are generally set forth in SEQ ID Nos. 229-282 and 288-300 and 346-351.

Further fragments of HlyA are set forth in SEQ ID Nos. 102-105 and 283-287. These comprise GG repeat sequences set forth in SEQ ID Nos. 1-3 and 21, 22 and 24.

All the afore-mentioned fragments may be used as first amino acid sequences in the sense of the present invention. All fragments set forth in SEQ ID Nos. 57-62 and 65-105, as well as 229-282 and 288-300 may be further truncated on their N- and/or C-terminal ends as long as at least one, at least two, three, four, five or all the GG repeats that are comprised in these fragments are retained. As disclosed above, it may be preferred that the sequences flanking the most N-terminal and most C-terminal GG repeat sequences are independently up to 60, up to 50, up to 40, up to 30 or up to 20 or up to 19, up to 18, up to 17, up to 16, up to 15, up to 14, up to 13, up to 12, up to 11, up to 10, up to 0, up to 8, up to 7, up to 6 or up to 5 amino acids in length. In view of the yields of the fused peptide or protein, it is generally advantageous to make the first amino acid sequence as short as possible without significantly impairing its beneficial influence on expression, stability and/or renaturation.

The invention further comprises, as first amino acid sequences, variants of all the afore-mentioned fragments of RTX proteins, including HlyA. These variants may have over their entire length, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99% or 99.5% sequence identity with the respective amino acid sequence as set forth in SEQ ID Nos. 34-53, 57-62, 65-105 and 117-159, with the proviso that the GG consensus sequences (i.e. the invariable residues of GGxGxDxUx, but not the variable x and U residues) comprised therein are invariable. In various embodiments, the exact GG repeat sequences comprised therein are invariable. In various embodiments and as explained above, such variants may be truncated versions of the amino acid sequences as set forth in SEQ ID Nos. 34-53, 57-62, 65-105, 117-159, 229-300, and 346-351 as long as at least one, at least 2, at least 3, at least 4, at least 5, at least 6 or all GG repeat sequences comprised in the respective sequence are retained.

The isolated polypeptide is typically a fusion protein. The second amino acid sequence that encodes the peptide or polypeptide of interest may be 10 to 1000 amino acids in length, for example 15 to 400 amino acids in length. In various embodiments, the second amino acid sequence is 10 to 500 amino acids in length, preferably 10 to 400, 300, or 200 amino acids in length. The lower limit may also be 12 or 15 amino acids and the upper limit, independently thereof, also 180 or 150 or 100 amino acids.

In various embodiments, the second amino acid sequence is N-terminal or C-terminal to the first amino acid sequence. In some embodiments, it may be C-terminal to the first amino acid sequence. The second amino acid sequence may be linked directly (via a peptide bond) or via a linker (amino acid; peptide) sequence to the N- or C-terminal end of the first amino acid sequence. If a linker sequence linking the two is present, the linker sequence is typically a peptide sequence of 1 to 30 amino acids in length. Said peptide linker sequence may have additional functionality and may, for example, comprise or consist of a protease recognition and cleavage site. Exemplary embodiments are described below.

In various embodiments, the isolated polypeptide has a total length of up to 700 amino acids, preferably up to 500 amino acids, more preferably up to 350 amino acids, most preferably up to 200 amino acids.

In some embodiments, the isolated polypeptide further comprises at least one third amino acid sequence, optionally at least one affinity tag.

In various embodiments, the isolated polypeptide has relative to a polypeptide comprising a first amino acid sequence not covered by the definition given herein, such as SEQ ID NO:26, an increased renaturation efficiency. In various embodiments, the isolated polypeptide has relative to a polypeptide comprising a first amino acid sequence comprising a C-terminal secretion signal, an increased renaturation efficiency.

In another aspect, the invention also relates to a nucleic acid, nucleic acid molecule or isolated nucleic acid molecule encoding the isolated polypeptide as described herein. In one aspect, said nucleic acid is part of a vector. One aspect thus features a (nucleic acid) vector comprising a nucleic acid molecule according to the invention. The vector may be an expression vector and may comprise additional nucleic acid sequences necessary to facilitate its function in a host cell. In various embodiments, the vector may be a plasmid.

One further aspect of the invention relates to a host cell comprising a nucleic acid molecule according to the invention or a vector according to the invention. The host cell may be a prokaryotic host cell, for example an E. coli cell.

In a still further aspect, the invention is directed to a method for the production of a polypeptide (isolated polypeptide) as described herein, comprising
(1) cultivating the host cell described herein under conditions that allow the expression of the polypeptide; and
(2) isolating the expressed polypeptide from the host cell.

The method may, in various embodiments, further comprise recovering the expressed peptide or protein from the host cell and/or the culture medium.

The method may in various embodiments also comprise recovering the expressed peptide or protein from the host cell in form of insoluble protein aggregates, in particular inclusion bodies (IBs). Such embodiments, in which the fusion protein is accumulated within the host cells, optionally in insoluble/denatured form, may be particularly advantageous alternatives of such methods.

In various embodiments, the methods may comprise a step of re-solubilizing the peptide/protein and/or reconstituting/refolding it under suitable conditions. Said step is also referred to herein as “renaturing” step. Such a step is described in more detail below in relation to methods for renaturing the isolated polypeptide.

In still another aspect, the present invention is therefore also directed to methods for renaturing the isolated polypeptide of the invention, wherein the method may comprise contacting, for example in form of re-solubilizing, the isolated polypeptide in a suitable medium, typically an aqueous medium comprising earth alkaline metal ions, including but not limited to calcium ions. It has been found that such a medium may trigger conformational changes in the GG repeats (comprised in the first amino acid sequence) that in turn then facilitate refolding/renaturation of the fused heterologous peptide or polypeptide (second amino acid sequence). This effect is independent of the type of fused peptide or polypeptide in that the conformational change of the first amino acid sequence effects the renaturation of the second amino acid sequence.

In various embodiments of the methods of the invention, the host cell is a prokaryotic cell, for example an E. coli cell. In various embodiments, the expression is performed in minimal culture medium. In various embodiments, the recombinant peptide or protein is purified using a method selected from affinity chromatography, ion exchange chromatography, reverse phase chromatography, size exclusion chromatography, and combinations thereof; and/or the method comprises treatment of the recombinant peptide or protein with a protease suitable for cleavage of a protease cleavage site within the recombinant peptide or protein and, optionally, said treatment with a protease is followed by purification of the recombinant peptide or protein.

In still another aspect, the present invention also relates to the use of an isolated polypeptide of the invention for facilitating the production of a recombinant peptide or protein. Said recombinant peptide of protein may be part of the isolated polypeptide, which is expressed as a fusion protein. In such embodiments, the first amino acid sequences disclosed herein may be used to facilitate renaturation/refolding of said recombinant peptide or polypeptide. Said recombinant peptide or protein is identical with the peptide or polypeptide of interest as referred to herein and may thus consist of the second amino acid sequence disclosed herein. As stated above, said use is preferably directed to recombinant polypeptides expressed as fusion proteins in a suitable expression system.

It is understood that all combinations of the above disclosed embodiments are also intended to fall within the scope of the present invention.

### BRIEF DESCRIPTION OF THE DRAWINGS

FIG. 1 shows renaturation efficiency (A) and amount of peptide formed (B). The renaturation efficiency (A) was determined from the set and the determined protein concentration after renaturation using the absorption at 280 nm and the corresponding extinction coefficient (Nanodrop). The integrated areas from HPLC/MS measurements at 205 nm reflect the amount of peptide formed after TEV-cleavage (B). Error bars indicate the deviation for n=2 or 3. On the X axis the different fragments are shown by indication of the deleted amino acids relative to SEQ ID NO:26. “wt” indicates that starting fragment of SEQ IDS NO:26 (HlyA1), used as first amino acid sequence. The second amino acid sequence and linker was identical for all constructs.

### DETAILED DESCRIPTION OF THE INVENTION

The terms used herein have, unless explicitly stated otherwise, the meanings as commonly understood in the art.

“At least one”, as used herein, relates to one or more, in particular 1, 2, 3, 4, 5, 6, 7, 8, 9, 10 or more.

“Isolated” as used herein in relation to a molecule means that said molecule has been at least partially separated from other molecules it naturally associates with or other cellular components. “Isolated” may mean that the molecule has been purified to separate it from other molecules and components, such as other proteins and nucleic acids and cellular debris, in particular those that accompany it due to its recombinant production in host cells.

“Nucleic acid” as used herein includes all natural forms of nucleic acids, such as DNA and RNA. Preferably, the nucleic acid molecules of the invention are DNA.

The term “peptide” is used throughout the specification to designate a polymer of amino acid residues connected to each other by peptide bonds. A peptide according to the present invention may have 2-100 amino acid residues. The terms “protein” and “polypeptide” are used interchangeably throughout the specification to designate a polymer of amino acid residues connected to each other by peptide bonds. A protein or polypeptide according to the present invention has preferably 100 or more amino acid residues.

The term “an N-terminal fragment” relates to a peptide or protein sequence which is in comparison to a reference peptide or protein sequence C-terminally truncated, such that a contiguous amino acid polymer starting from the N-terminus of the peptide or protein remains. In some embodiments, such fragments may have a length of at least 30 amino acids, at least 50 amino acids or at least 70 amino acids.

The term “a C-terminal fragment” relates to a peptide or protein sequence which is in comparison to a reference peptide or protein sequence N-terminally truncated, such that a contiguous amino acid polymer starting from the C-terminus of the peptide or protein remains. In some embodiments, such fragments may have a length of at least 30 amino acids, at least 50 amino acids or at least 70 amino acids.

The term “fusion protein” as used herein concerns two or more peptides and proteins which are N- or C-terminally connected to each other, typically by peptide bonds, including via an amino acid/peptide linker sequence. Such fusion proteins may be encoded by two or more nucleic acid sequences which are operably fused to each other. In certain embodiments, a fusion protein refers to at least one peptide or protein of interest C-terminally or N-terminally fused to a first amino acid sequence according to the invention. In various embodiments, the peptide or protein of interest is fused to the C-terminus of the first amino acid sequence, optionally via a linker sequence.

“Stability”, as used herein in relation to the polypeptides of the invention, primarily relates to resistance to proteolytic degradation, which is a commonly encountered issue, in particular in conditions of high cell density and/or high protein yields.

“Solubility”, as used herein in relation to the polypeptides of the invention, primarily relates to solubility in the cytoplasm and/or culture medium so that the polypeptide can be successfully secreted and purified from the cultivation medium.

“Renaturation” and “renaturation efficiency”, as used herein in relation to the polypeptides of the invention, primarily relates to the possibility to isolate the polypeptide in denatured/unfolded form, for example from insoluble protein aggregates, such as inclusion bodies, and induce its proper folding to the active, three-dimensional conformation. The efficiency may be given as the mass of successfully renatured protein relative to the total mass of all expressed protein, including insoluble protein, in percent. The refolding may be effected in special refolding buffers including earth alkaline metal ions, in particular calcium ions, to induce refolding of the first amino acid sequence, which in turn facilitates renaturation of the complete fusion construct into its functional conformation. “Renaturing” and “refolding” are used interchangeably herein and generally relate to the process of folding a given protein that is unfolded or denatured such that it adopts its functional three-dimensional conformation.

Generally, the skilled person understands that for putting the present invention into practice any nucleotide sequence described herein may comprise an additional start and/or stop codon or that a start and/or stop codon included in any of the sequences described herein may be deleted, depending on the nucleic acid construct used. The skilled person will base this decision, e.g., on whether a nucleic acid sequence comprised in the nucleic acid molecule of the present invention is to be translated and/or is to be translated as a fusion protein. In various embodiments, the isolated polypeptides of the invention additionally comprise the amino acid M on the N-terminus.

Determination of the sequence identity of nucleic acid or amino acid sequences can be done by a sequence alignment based on well-established and commonly used BLAST algorithms (See, e.g. Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, S.3389-3402). Such an alignment is based on aligning similar nucleotide or amino acid sequences stretches with each other. Another algorithm known in the art for said purpose is the FASTA algorithm. Alignments, in particular multiple sequence comparisons, are typically done by using computer programs. Commonly used are the Clustal series (See, e.g., Chenna et al. (2003): Multiple sequence alignment with the Clustal series of programs. Nucleic Acid Research 31, 3497-3500), T-Coffee (See, e.g., Notredame et al. (2000): T-Coffee: A novel method for multiple sequence alignments. J. Mol. Biol. 302, 205-217) or programs based on these known programs or algorithms.

Also possible are sequence alignments using the computer program Vector NTI® Suite 10.3 (Invitrogen Corporation, 1600 Faraday Avenue, Carlsbad, CA, USA) with the set standard parameters, with the AlignX module for sequence comparisons being based on the ClustalW. If not indicated otherwise, the sequence identity is determined using the BLAST algorithm.

Such a comparison also allows determination of the similarity of the compared sequences. Said similarity is typically expressed in percent identify, i.e. the portion of identical nucleotides/amino acids at the same or corresponding (in an alignment) sequence positions relative to the total number of the aligned nucleotides/amino acids. For example, if in an alignment 90 amino acids of a 100 aa long query sequence are identical to the amino acids in corresponding positions of a template sequence, the sequence identity is 90%. The broader term “homology” additionally considers conserved amino acid substitutions, i.e. amino acids that are similar in regard to their chemical properties, since those typically have similar chemical properties in a protein. Accordingly, such homology can be expressed in percent homology. If not indicated otherwise, sequence identity and sequence homology relate to the entire length of the aligned sequence.

In the context of the present invention, the feature that an amino acid position corresponds to a numerically defined position in a reference sequence means that the respective position correlates to the numerically defined position in said reference sequence in an alignment obtained as described above.

The hemolysin (Hly) secretion system is a protein secretion system which mostly occurs in gram-negative bacteria. This secretion system belongs to the family of type I secretion systems which transport their substrates in an ATP driven manner in a single step from the cytosol to the extracellular space without an intermediate station in the periplasm. The Hly secretion system comprises hemolysin B (HlyB) which represents an ATP-binding cassette (ABC) transporter, the membrane fusion protein hemolysin D (HlyD), and the universal outer membrane protein TolC. The 110 kDa hemolytic toxin hemolysin A (HlyA) is a transport substrate of the Hly secretion system. On genetic level, the components necessary for hemolysin A-specific secretion are organized in an operon structure. The nucleic acid sequence encoding for hemolysin C (HlyC) also forms part of this operon but is not required for HlyA secretion through the Hly secretion system. HlyC catalyzes acylation of HlyA which renders HlyA hemolytic. HlyA is a protein which consists of 1024 amino acid residues and requires for its export via the Hly secretion system its C-terminus, comprising about 40-60 amino acids. Furthermore, HlyA is characterized in that it comprises N-terminally to the 40-60 C-terminal amino acids a domain comprising several glycine rich (GG) repeats (GGXGXDXXX, wherein X can be any amino acid), with the amino acid sequences thereof set forth in SEQ ID Nos. 1-3, 21, 22 and 24. The Hly secretion system and its T1 SS allocrit HlyA are used as an exemplary embodiment herein to demonstrate the functionality of the inventive concept. However, it is to be understood that the invention is not limited to these constructs derived from HlyA, since similar results have also be obtained with the GG repeat-containing sequences of other RTX proteins.

“GG repeat”, “GG repeat sequence” and “glycine rich repeat”, as used herein interchangeably, are the characteristic of the repeats in toxin (RTX) toxin family and have the consensus sequence disclosed herein. The glycine rich repeats bind Ca²⁺ which induces their folding. Hence, in absence of earth alkaline metal ions, in particular Ca²⁺, the domain comprising the glycine rich repeats is unstructured.

The present invention is based on the inventor's surprising finding that certain variants and fragments of RTX proteins and derived amino acid sequences that comprise at least one or more GG repeats may be fused to a peptide/protein of interest with the result that the obtained fusion protein can efficiently be renaturated due to the presence of the GG repeats, with the renaturation efficiency being significantly higher than that obtained with the full RTX protein, longer fragments thereof, fragments that lack the GG repeats, or fragments thereof that comprise the secretion signal on the C-terminus. In particular the finding that the GG repeats as such are sufficient for the desired effect and that the secretion signal is disadvantageous for the desired properties have important impact on the design of fusion proteins for heterologous expression of peptides and polypeptides.

The present invention is directed to such fusion proteins that comprise a variant or fragment of an RTX protein that comprises at least one GG repeat as a functional tag that imparts the desired properties of increased expression, stability and/or renaturation efficiency to the fusion protein as a first amino acid sequence, while the peptide(s) and/or protein(s) of interest, i.e. the cargo that is to be produced, is the second amino acid sequence. These fusion proteins are also more generally referred to herein as polypeptides that are isolated after production. These fusion proteins and isolated polypeptides are artificial constructs, since the first and second amino acid sequences are typically heterologous to each other and/or the fusion construct has been genetically engineered.

As it is desirable to have maximum yields of the peptide or protein of interest, the first amino acid sequences of the invention are preferably as short as possible, generally with a maximum length of about 250 amino acids, typically about 200 amino acids. Together with optional linker and protease cleavage sites, the total length of the part of the fusion protein not being the peptide or protein of interest is typically limited to about 250 amino acids or less. In various embodiments, it may be desirable to use first amino acid sequence significantly short than the upper limit of 200 amino acids, such as up to 190, up to 180, up to 170, up to 160, up to 150, up to 140, up to 130, up to 120, up to 110, up to 100, up to 90, up to 85, up to 80, up to 75, up to 70, up to 65, up to 60, up to 55, up to 45, or up to 40 amino acids in length. Since the GG repeat consensus sequence is 9 amino acids in length, even a first amino acid sequence of only 30 amino acids could still accommodate 3 GG repeat sequences. In various embodiments, the first amino acid sequence is long enough to accommodate 4, 5, or 6 GG repeats, i.e. is at least 36, at least 45 or at least 54 amino acids in length.

The at least one GG repeat sequence has the general consensus sequence GGxGxDxUx, wherein x can be any amino acid and U is a hydrophobic, large amino acid selected from F, I, L, M, W and Y. It may be preferred that the first amino acid sequence comprises at least 2, at least 3, at least 4, at least 5 or at least 6 GG repeat sequences of this general consensus sequence GGxGxDxUx, since it has been found that the desirable properties are particularly pronounced if two or three GG repeat sequences are present. These may be arranged directly adjacent to each other, i.e. linked directly by a peptide bond or connected by short linker sequences. These linker sequences may be amino acid sequences of 1 to 25 amino acids, preferably 1 to 20 or 1 to 15 or 1 to 10 or 1 to 9 or 1 to 8 or 1 to 7 or 1 to 6 or 1 to 5 or 1 to 4 or 1 to 3 or 1-2 amino acids in length. These are not limited with respect to their sequences, but, in various embodiments, it may be preferred to keep these linking sequences short, for example of only 12, 11, 10, 9, 8, 7, 6, 5, 4, 3, 2 or 1 amino acid in length. If two or more GG repeat sequences are contained in the first amino acid sequence it may be preferred that at least two, or depending on the number present, at least three, at least 4, at least 5, at least 6 or all of them are directly linked to each other by a peptide bond. It has been found, for example, that in some embodiments constructs where the first amino acid sequence comprises 3, 4, 5 or 6 GG repeat sequences, with these sequences directly linked to each other, perform particularly well.

In various embodiments, the U in the consensus sequence is selected from F, I, L, M and Y.

In various embodiments, x is typically selected from the 20 proteinogenic amino acids G, A, V, L, I, F, C, M, P, R, K, H, N, Q, D, E, S, T, W, and Y. If not indicated otherwise, it is generally preferred that any amino acid not specifically defined is selected from these 20 proteinogenic amino acids. In various embodiments, x may be any one of the above amino acids with the exception of proline. In even more specific embodiments, the consensus sequence may be GGX¹GX²DX³UX⁴, wherein X¹, X², X³ and X⁴ may be any amino acid with the exception of P.

In various embodiments, X¹ is selected from G, A, L, V, I, M, F, S, T, Q; Y, K, R, D, E, N, and Y, for example from G, A, E, S, T, Q, L, R and D, or from G, A, E, S and T. In various embodiments, X² is selected from N, D, A, G, S, H, T, E, M, R and H, for example from N, D, A, S, and H, or from N, D, A, and S. In various embodiments, X³ is selected from A, K, V, L, I, F, M, R, Y, S, L, T, V, Q, N, D, E, and H, for example from T, R, V, L, S, I, A, Y, Q, D and H, or from T, R, V, L, S and I, or from T, R and V. In various embodiments, X⁴ is selected from W, K, R, Y, V, L, T, H, D, A, M, E, F, I, S, N and Q, for example from V, L, I, F, S, R, N, Y and T. These embodiments of X¹-X⁴ may be combined with each other.

The first amino acid sequence may comprise the general structure, from N- to C-terminus,
(i) GGR1-Linker-GGR2, or
(ii) GGR1-Linker-GGR2-Linker-GGR3; or
(iii) GGR1-Linker-GGR2-Linker-GGR3-Linker-GGR4; or
(iv) GGR1-Linker-GGR2-Linker-GGR3-Linker-GGR4-Linker-GGR5; or
(v) GGR1-Linker-GGR2-Linker-GGR3-Linker-GGR4-Linker-GGR5-Linker-GGR6
(vi) GGR1-Linker-GGR2-Linker-GGR3-Linker-GGR4-Linker-GGR5-Linker-GGR6-Linker-GGR7
(vii) GGR1-Linker-GGR2-Linker-GGR3-Linker-GGR4-Linker-GGR5-Linker-GGR6-Linker-GGR7-Linker-GGR8
   wherein GGR1, GGR2, GGR3, GGR4, GGR5, GGR6, GGR7 and GGR8 are GG repeats of the consensus sequence GGxGxDxUx and wherein each “Linker” is independently either a peptide bond or an amino acid sequence of 1 to 25 amino acids, preferably 1 to 20 or 1 to 15 amino acids in length. In various preferred embodiments, the Linker does not comprise a GG repeat sequence. As disclosed above, it may be preferred that at least one Linker is a peptide bond. It is understood that also constructs with more than 8 GG repeats are covered, with the ninth and further GG repeat also linked via a “Linker” as herein defined.

The GG repeat sequences of the first amino acid sequence and more particularly the general structures given above as (i)-(v) may be flanked by additional N- and/or C-terminal amino acid sequences that may be 1 to 100 amino acids in length, preferably 1 to 80, 1 to 70, 1 to 60, 1 to 50, 1 to 40, 1 to 30, 1 to 20, 1 to 10 or 1 to 5 amino acids in length. As disclosed above, it may be preferred that the sequences flanking the most N-terminal GG repeat sequence on the N-terminus are up to 60, up to 50, up to 40, up to 30 or up to 20, up to 19, up to 18, up to 17, up to 16, up to 15, up to 14, up to 13, up to 12, up to 11, up to 10, up to 9, up to 8, up to 7, up to 6, up to 5 or 1, 2, 3, or 4 amino acids in length. The same applies to the amino acids flanking the most C-terminal GG repeat on the C-terminus. In view of the yields of the fused peptide or protein, it is generally advantageous to make the first amino acid sequence as short as possible without significantly impairing its beneficial influence on expression, stability and/or renaturation.

Exemplary GG repeats that may be used in the first amino acid sequences of the invention in any combination or order are the following: GGAGNDSYF (SEQ ID NO:8), GGAGNDIIY (SEQ ID NO:10), GGAGADTFV (SEQ ID NO:12), GGAGNDLME (SEQ ID NO:14), GGAGNDIIR (SEQ ID NO:18), GGAGDDTFV (SEQ ID NO:20), GGAGNDYLN (SEQ ID NO:23), GGAGADVLS (SEQ ID NO:63). GGAGSDYLS (SEQ ID NO:165), GGAGADQLF (SEQ ID NO:166), GGAGDDTTV (SEQ ID NO:167), GGAGADDLT (SEQ ID NO:168), GGAGADNFI (SEQ ID NO:169), GGAGNDEVH (SEQ ID NO:170), GGAGNDYLS (SEQ ID NO:171), GGAGNDSLF (SEQ ID NO:172), GGAGFDILI (SEQ ID NO:173), GGAGNDVAF (SEQ ID NO:174), GGAGNNDIYH (SEQ ID NO:175), GGAGADSFV (SEQ ID NO:176), GGAGADALY (SEQ ID NO:177), GGAGSDAIV (SEQ ID NO:178), GGAGEDTFR (SEQ ID NO:179), GGAGHDRLA (SEQ ID NO:180), GGAGADTFV (SEQ ID NO:181), GGAGDDQLS (SEQ ID NO:182), GGAGDDVLE (SEQ ID NO:183), GGAGTDHLD (SEQ ID NO:184), GGAGNDRID (SEQ ID NO:185), GGAGADQLW (SEQ ID NO:186), GGAGNDTFV (SEQ ID NO:187), GGAGGDLLD (SEQ ID NO:188), GGAGEDSFR (SEQ ID NO:189), GGAGNDLME (SEQ ID NO:190), GGAGMDALH (SEQ ID NO:191), GGAGTDTLV (SEQ ID NO:192), GGAGADTLY (SEQ ID NO:193), GGAGADELT (SEQ ID NO:194), GGDGADRIS (SEQ ID NO:17), GGAGADRLD (SEQ ID NO:368), GGDGNDKLI (SEQ ID NO:22), GGDGDDELQ (SEQ ID NO:24), GGDGNDVLL (SEQ ID NO:195), GGDGNDSLV (SEQ ID NO:367), GGDGADLLF (SEQ ID NO:196), GGDGTDFLL (SEQ ID NO:197), GGEGDDLLK (SEQ ID NO:2), GGEGHDFVS (SEQ ID NO:198), GGEGDDRVY (SEQ ID NO:199), GGEGADLLF (SEQ ID NO:200), GGEGRDSLY (SEQ ID NO:227), GGEGNDHLR (SEQ ID NO:201), GGEGADRLI (SEQ ID NO:202), GGFGNDEVN (SEQ ID NO:203), GGGGDDIIV (SEQ ID NO:6), GGGGGDTLW (SEQ ID NO:11), GGGGHDRMQ (SEQ ID NO:19), GGGGSDIMR (SEQ ID NO:204), GGGGNDILI (SEQ ID NO:205), GGGGNDRLE (SEQ ID NO:206), GGGGSDMFV (SEQ ID NO:207), GGKGNDKLY (SEQ ID NO:1), GGKGDDYLE (SEQ ID NO:7), GGLGDDHLV (SEQ ID NO:15), GGLGSDVLD (SEQ ID NO:208), GGLGSDQLF (SEQ ID NO:209), GGLGADTLI (SEQ ID NO:210), GGLGSDAFA (SEQ ID NO:358), GGMGADELT (SEQ ID NO:211), GGNGDDQLY (SEQ ID NO:21), GGNGVDLAN (SEQ ID NO:369), GGQGNDVFV (SEQ ID NO:13), GGQGRDQLH (SEQ ID NO:212), GGRGSDLLI (SEQ ID NO:16), GGRGSDIFA (SEQ ID NO:213), GGRGSDLLD (SEQ ID NO:214), GGSGNDLLI (SEQ ID NO:9), GGSGNDRLI (SEQ ID NO:215), GGSGNDRLD (SEQ ID NO:216), GGSGNDDLS (SEQ ID NO:217), GGSGDDRYQ (SEQ ID NO:218), GGSGSDTFV (SEQ ID NO:219), GGTGNDRLW (SEQ ID NO:4), GGTGADIFV (SEQ ID NO:5), GGTGNDLVS (SEQ ID NO:220), GGTGGDTLS (SEQ ID NO:221), GGTGHDTLI (SEQ ID NO:222), GGTGSDRLV (SEQ ID NO:223), GGTGNDTYI (SEQ ID NO:224), GGTGRDVFL (SEQ ID NO:225), GGVGADTMT (SEQ ID NO:226), and GGYGNDIYR (SEQ ID NO:3). This means that if a given first amino acid sequence comprises 3 GG repeats, these may be, from N- to C-terminus, SEQ ID Nos:1, 2 and 3 or SEQ ID Nos: 2, 3 and 1 or SEQ ID Nos: 3, 4 and 5 etc. In any case, if two or more GG repeat sequences are comprised in the first amino acid sequence, each may be selected independently from those listed above.

In various embodiments, the at least one GG repeat sequence is selected from any one of GGAGNDIIR (SEQ ID NO:18), GGAGDDTFV (SEQ ID NO:20), GGAGSDYLS (SEQ ID NO:165), GGAGADQLF (SEQ ID NO:166), GGAGADDLT (SEQ ID NO:168), GGAGADNFI (SEQ ID NO:169), GGAGNDEVH (SEQ ID NO:170), GGAGNDYLS (SEQ ID NO:171), GGDGADRIS (SEQ ID NO:17), GGDGNDVLL (SEQ ID NO:195), GGDGADLLF (SEQ ID NO:196), GGEGHDFVS (SEQ ID NO:198), GGEGDDRVY (SEQ ID NO:199), GGEGADLLF (SEQ ID NO:200), GGEGRDSLY (SEQ ID NO:227), GGFGNDEVN (SEQ ID NO:203), GGGGHDRMQ (SEQ ID NO:19), GGLGDDHLV (SEQ ID NO:15), GGLGSDAFA (SEQ ID NO:358), GGLGSDVLD (SEQ ID NO:208), GGLGSDQLF (SEQ ID NO:209), GGRGSDLLI (SEQ ID NO:16), GGRGSDIFA (SEQ ID NO:213), GGTGNDLVS (SEQ ID NO:220), GGTGGDTLS (SEQ ID NO:221), and GGVGADTMT (SEQ ID NO:226). If two or more GG repeat sequences are comprised in the first amino acid sequence, each may be selected independently from those listed above.

In various embodiments, the first amino acid sequence comprises any one, two, three, four, five, six, seven eight, nine or more of the above-listed GG repeat sequences that may be arranged in any order. In various embodiments such an arrangement is artificial in that the combined GG repeats do not naturally occur in combination. Furthermore, in various embodiments, the GG repeats are arranged as detailed above in that they are directly linked to each other by a peptide bond and/or a “Linker”.

In some embodiments, the first amino acid sequence is a fragment derived from a naturally occurring protein. In such embodiments, where the isolated polypeptide comprises, i.e. the first amino acid sequence comprised therein, the general structure of any one of (i)-(v) above, the arrangement of the GG repeat sequences may be as follows:
a) GGR1 is GGKGNDKLY (SEQ ID NO:1) and/or GGR2 is GGEGDDLLK (SEQ ID NO:2) and/or GGR3 is GGYGNDIYR (SEQ ID NO:3);
b) GGR1 is GGTGNDRLW (SEQ ID NO:4) and/or GGR2 is GGAGADVLS (SEQ ID NO:63) and/or GGR3 is GGTGADIFV (SEQ ID NO:5);
c) GGR1 is GGGGDDIIV (SEQ ID NO:6) and/or GGR2 is GGKGDDYLE (SEQ ID NO:7) and/or GGR3 is GGAGNDSYF (SEQ ID NO:8);
d) GGR1 is GGSGNDLLI (SEQ ID NO:9) and/or GGR2 is GGAGNDIIY (SEQ ID NO:10) and/or GGR3 is GGGGGDTLW (SEQ ID NO:11) and/or GGR4 is GGAGADTFV (SEQ ID NO:12);
e) GGR1 is GGQGNDVFV (SEQ ID NO:13) and/or GGR2 is GGAGNDLME (SEQ ID NO:14); or
f) GGR1 is GGLGDDHLV (SEQ ID NO:15) and/or GGR2 is GGRGSDLLI (SEQ ID NO:16) and/or GGR3 is GGDGADRIS (SEQ ID NO: 17) and/or GGR4 is GGAGNDIIR (SEQ ID NO:18) and/or GGR5 is GGGGHDRMQ (SEQ ID NO: 19) and/or GGR6 is GGAGDDTFV (SEQ ID NO:20); or
g) GGR1 is SEQ ID NO:21 and/or GGR2 is SEQ ID NO:22 and/or GGR3 is SEQ ID NO:24;
h) GGR1 is GGKGNDKLY (SEQ ID NO:1) and GGR2 is GGEGDDLLK (SEQ ID NO:2) and GGR3 is GGYGNDIYR (SEQ ID NO:3);
i) GGR1 is GGTGNDRLW (SEQ ID NO:4) and GGR2 is GGAGADVLS (SEQ ID NO:63) and GGR3 is GGTGADIFV (SEQ ID NO:5);
j) GGR1 is GGGGDDIIV (SEQ ID NO:6) and GGR2 is GGKGDDYLE (SEQ ID NO:7) and GGR3 is GGAGNDSYF (SEQ ID NO:8);
k) GGR1 is GGSGNDLLI (SEQ ID NO:9) and GGR2 is GGAGNDIIY (SEQ ID NO:10) and GGR3 is GGGGGDTLW (SEQ ID NO:11) and GGR4 is GGAGADTFV (SEQ ID NO:12);
l) GGR1 is GGQGNDVFV (SEQ ID NO:13) and GGR2 is GGAGNDLME (SEQ ID NO:14); or
m) GGR1 is GGLGDDHLV (SEQ ID NO: 15) and GGR2 is GGRGSDLLI (SEQ ID NO:16) and GGR3 is GGDGADRIS (SEQ ID NO:17) and GGR4 is GGAGNDIIR (SEQ ID NO:18) and GGR5 is GGGGHDRMQ (SEQ ID NO:19) and GGR6 is GGAGDDTFV (SEQ ID NO:20); or
n) GGR1 is SEQ ID NO:21 and GGR2 is SEQ ID NO:22 and GGR3 is SEQ ID NO: 24.
o) GGR1 is SEQ ID NO:221 and/or GGR2 is SEQ ID NO: 195 and/or GGR3 is SEQ ID NO: 168 and/or GGR4 is SEQ ID NO:226 and/or GGR5 is SEQ ID NO: 169; or
p) GGR1 is SEQ ID NO: 170 and/or GGR2 is SEQ ID NO:227 and/or GGR3 is SEQ ID NO:208 and/or GGR4 is SEQ ID NO: 171 and/or GGR5 is SEQ ID NO:209 and/or GGR6 is SEQ ID NO: 196 and/or GGR7 is SEQ ID NO:213; or
q) GGR1 is SEQ ID NO: 198 and/or GGR2 is SEQ ID NO:203 and/or GGR3 is SEQ ID NO: 199 and/or GGR4 is SEQ ID NO:220 and/or GGR5 is SEQ ID NO: 165 and/or GGR6 is SEQ ID NO: 166 and/or GGR7 is SEQ ID NO:200 and/or GGR8 is SEQ ID NO:358.

In various embodiments of a) to q), the isolated polypeptide may lack any one or more of GGR1 to GGR6 as long as at least 1, preferably at least 2 or at least 3 GG repeat sequences remain. Furthermore, any two or more of a) to q) may be combined or repeated in the first amino acid sequence, as long as the size limitation is met.

In various embodiments, the first amino acid sequence corresponds to any of the GG repeat core sequences set forth in SEQ ID Nos. 235, 241, 246, 251, 252, 256, 261, 269, 274, 278, 282, 289, 291, 293, 295, 297, 298, 300, 359, 360 and 364. These are the GG repeat core sequences of RTX1, RTX9, RTX12, RTX18, RTX21, RTX22, RTX30, RTX33, RTX28, RTX24, RTX19, RTX16, RTX26, RTX3, RTX20, RTX31, RTX14, RTX32 and RTX29. In various embodiments, the first amino acid sequence comprises, consists essentially of or consists of any of these sequences, in particular those with the amino acid sequence set forth in SEQ ID Nos. 235, 278, 241, 246, 282, 251, 252, 256, 359, and 364. These are the GG core sequences of RTX1, RTX12, RTX16, RTX18, RTX22, RTX21, RTX26, RTX30 and RTX33. In some embodiments, the first amino acid sequence may comprise the GG repeat core sequences of, for example, RTX1, RTX12, RTX16, RTX18, RTX20, RTX21, RTX22, RTX24, RTX30 and RTX32. It has been found that the GG repeat containing core sequences and fragments of RTX 12 are particularly advantageous and can accommodate and effectively renature second amino acid sequences that are short peptides with a length of up to 100 amino acids, for example up to 80 or up to 70 amino acids, as well as longer polypeptides with a length of more than 100 amino acids. The fragments of RTX12 are thus highly versatile and can also be used to design artificial GG repeat sequences that function similarly well or better than their native templates. Further, it has been found that the GG repeat containing sequences and fragments of RTX1, RTX16, RTX 18 also show good performance with a variety of peptides and polypeptides of interest.

Also encompassed are fragments of the afore-mentioned sequences as long as they comprise at least one, preferably at least two GG repeat sequences. Also contemplated are variants where any GG repeat in one of these sequences is replaced by a GG repeat from another of these sequences.

In various embodiments, the first amino acid sequence does not comprise a secretion signal, i.e. an amino acid sequence that facilitates secretion from the host cell into the medium. This may mean that the first amino acid sequence does not comprise the C-terminal sequence TTSA (SEQ ID NO:25). It may also mean that the secretion signal is rendered non-functional by a truncation, deletion or amino acid substitution. As detailed below, this may mean that the first amino acid sequence lacks the C-terminal amino acids of the native protein it is derived from, typically lacks at least the 40 to 60 C-terminal amino acids. “Lacking at least 40 C-terminal amino acids” means that the amino acids 1-40, counting from the C-terminal end and the C-terminal amino acid being amino acid 1, are missing.

In various embodiments, the first amino acid sequence may be a fragment of a naturally occurring RTX protein, such as those disclosed herein below. Such fragments are typically at least 30 amino acids in length with a maximum length of 200 amino acids and comprise at least one GG repeat sequence, preferably two or three GG repeat sequences, as defined herein. In various embodiments, said fragments do not retain a functional, typical C-terminal, secretion signal, e.g. at least the C-terminal 40, preferably 50 or 60 amino acids (i.e. typically the 40 to 60 C-terminal amino acids of the native, naturally occurring protein) are not part of the fragment. The fragments are thus typically N-terminal fragments that may additionally lack most of the N-terminus and retain only the amino acid sequences in direct vicinity of the retained GG repeats. These retained sequences adjacent to the core GG repeat sequence are also referred to herein as “flanking sequences”.

These RTX proteins may be allocrits of a T1 SS and may be selected from the group consisting of, without limitation, HlyA, CyaA, EhxA, LktA, PILktA, PasA, PvxA, MmxA, LtxA, ApxIA, ApxIIA, ApxIIIA, ApxIVA, ApxI, Apx11, AqxA, VcRtxA, VvRtxA, MbxA, RTX cytotoxin, RtxL1, RtxL2, FrhA, LipA, TliA, PrtA, PrtSM, PrtG, PrtB, PrtC, AprA, AprX, ZapA, ZapE, Sap, HasA, colicin V, LapA, ORF, RzcA, RtxA, XF2407, XF2759, RzcA, RsaA, Crs, CsxA, CsxB, SlaA, SwmA, S111951, NodO, PlyA, PlyB, FrpA, FrpC, RTX1, RTX3, RTX7, RTX8, RTX9, RTX12, RTX14, RTX16, RTX18, RTX19, RTX20, RTX21, RTX22, RTX24, RTX26, RTX28, RTX29, RTX30, RTX31, RTX32, and RTX33. The amino acid sequences of the recited RTX proteins are available in the Uni Prot database under the following accession numbers:
RTX1 (SCP-domain containing protein, Aromatoleum aromaticum strain EbN1; NCBI Reference WP_011239843.1; UniProt Accession No. Q5NXL6),
RTX3 (Serralysin (Caulobacter sp. strain K31); UniProt Accession No. B0T558),
RTX7 (uncharacterized protein of Pelobacter propionicus; UniProt Accession No. A1ARK7; NCBI Reference WP_011736233.1),
RTX8 (Metallopeptidase AprX (Pseudomonas fluorescens strain ATCC BAA-477/NRRL B-23932/Pf-5); UniProt Accession No. Q4KDU3; NCBI Reference WP_011060780.1),
RTX9 (glycosyl hydrolase family 16, hemolysin-type calcium-binding repeat protein (Rhodobacter sphaeroides strain ATCC 17023/DSM 158/JCM 6121); UniProt Accession No. Q3J0J9; NCBI Reference WP_011338282.1),
RTX12 (endonuclease/exonuclease/phosphatase (Caulobacter sp. strain K31); UniProt Accession No. B0T829; NCBI Reference WP_012287684.1),
RTX14 (Glycoside hydrolase family 16 (Caulobacter sp. strain K31); UniProt Accession No. B0T438),
RTX16 (lipase class 3 (Chlorobium phaeobacteroides strain DSM266); UniProt Accession No. A1BIU7; NCBI Reference WP_011746110.1),
RTX18 (animal haem peroxidase (Leptotrix cholodnii srain ATCC 51168/LMG 8142/SP-6); UniProt Accession No. B1Y442; NCBI Reference WP_012347322.1),
RTX19 (putative outer membrane adhesin like protein (Magnetococcus marinus strain ATCC BAA-1437/JCM 17883/MC-1); UniProt Accession No. A0L6L9; ENA Reference ABK43612.1),
RTX20 (putative outer membrane adhesin like protein (Magnetococcus marinus strain ATCC BAA-1437/JCM 17883/MC-1); UniProt Accession No. A0LDY6; NCBI Reference WP_011715232.1),
RTX21 (Glycerophosphoryl diester phosphodiesterase (Methylobacterium nodulans (strain LMG 21967/CNCM I-2342/ORS 2060); UniProt Accession No. B8ICD8; EMBL Reference ACL55526.1),
RTX22 (5′ nucleotidase domain protein (Methylobacterium sp. strain 4-64); UniProt Accession No. B0UQ63; NCBI Reference WP_012331605.1),
RTX24 (polyurethanase A (Pseudomonas fluorescens strain ATCC BAA-477/NRRL B 23932/Pf-5); UniProt Accession No. Q4KBS6; NCBI Reference WP_011061486.1),
RTX26 (extracellular alkaline metalloprotease AprA (Pseudomonas fluorescens strain ATCC BAA-477/NRRL B-23932/Pf-5); UniProt Accession No. Q4KBR8; NCBI Reference WP_011061494.1),
RTX28 (hemolysin type calcium-binding protein (Rhodobacter sphaeroides strain ATCC 17023/DSM 158/JCM 6121); UniProt Accession No. Q3IVG4; NCBI Reference WP_011331290.1),
RTX29 (neutral zinc metallopeptidase (Rhodobacter sphaeroides strain ATCC 17023/DSM 158/JCM 6121); UniProt Accession No. Q3J1D3; NCBI Reference WP_011338088.1),
RTX30 (glycoside hydrolase family 16 (Rhodobacter sphaeroides strain ATCC 17029/ATH 2.4.9); UniProt Accession No. A3PLQ2; NCBI Reference WP_011841485.1),
RTX31 (peptidase domain protein (Sinorhizobium medicae strain WSM419); UniProt Accession No. A6UK74; NCBI Reference WP_011970163.1),
RTX32 (Serralysin (Sinorhizobium medicae strain WSM419); UniProt Accession No. A6UK83; NCBI Reference WP_011970172.1), and
RTX33 (hemolysin type calcium-binding region (Verminephrobacter eiseniae strain EF01-2); UniProt Accession No. A1WL15; NCBI Reference WP_011810323.1).

As disclosed herein, these proteins are not used as such as the first amino acid sequence, as they are typically more than 200 amino acids in length, but rather fragments thereof that comprise 1 or more, preferably at least two, GG repeat sequences and, preferably, do not comprise the (C-terminal) secretion signal are used. It is understood that if the first amino acid sequence is a fragment of any such polypeptide or protein, the isolated polypeptide does not comprise the missing parts or other parts of said polypeptide or protein, for example as linker or second amino acid sequence. This means that if it is herein disclosed that the first amino acid sequence has a given length or is a fragment of a natural polypeptide or protein, the isolated polypeptide of the invention does not comprise the missing parts or other parts of said polypeptide or protein that has been used as a source for the first amino acid sequence.

In various exemplary embodiments, such fragments may be derived from a known C-terminal fragment of HlyA, namely HlyA1 (SEQ ID NO:26). Said known fragment comprises 3 GG repeats having the amino acid sequences set forth in SEQ ID Nos. 1-3 in positions 11-19, 29-37, and 38-46 using the positional numbering of SEQ ID NO:26. These GG repeats, namely 1, 2 or all 3, are preferably retained in the fragments thereof that are used as the first amino acid sequence in accordance with the present invention. It has however been found that also fragments where only two of these GG repeats, in particular the first two of SEQ ID Nos. 1-2, are retained perform well or even better than longer fragments. In such embodiments, the first amino acid sequence (and also the total isolated polypeptide) comprises less than 218 continuous amino acids of the amino acid sequence set forth in SEQ ID NO:26. In such embodiments, the first amino acid sequence is derived from the amino acid sequence set forth in SEQ ID NO:26 by any one or more of an N-terminal truncation, a C-terminal truncation or a deletion of one or more amino acids, in particular a C-terminal truncation. In such embodiments, the first amino acid sequence may be at least 50 amino acids in length, preferably at least 60, 70, 80, 90, 100, 110, 120, 130, 140, 150, 160, 170, 180, 190, 200, 210, 211, 212, 213, 214, 215, 216, or 217 amino acids long. In various embodiments, the fragment of SEQ ID NO:26 has a C-terminal truncation, for example up to 30, up to 35, up to 40, up to 45, up to 50, up to 55 or up to 60 amino acids, or may lack the amino acids corresponding to amino acids 135-218, 130-218, 125-218, 120-218, 115-218, 110-218, 105-218, 100-218, 95-218, 90-218, 85-218, 80-218, 75-218, 70-218, 65-218, 60-218, 55-218, 50-218, 45-218 or 40-218 of SEQ ID NO:26, the amino acid sequence of such fragments being set forth in SEQ ID Nos. 34-53, respectively. Also encompassed are all other deletions up to residue 40, preferably up to residue 46, using the positional numbering of SEQ ID NO:26. In various embodiments, any one or more of the amino acids up to the position corresponding to position 109 of SEQ ID NO:26 (starting from 218 and counting backwards) may be deleted. Such deletions may cover the amino acid residues at the positions corresponding to positions 185-218, positions 165-218, positions 135-218 or positions 110-218 of SEQ ID NO:26. Also encompassed are any deletions of ranges between those recited above, such as 184-218, 183-218, 182-218, 181-218, 180-218, 179-218, 178-218, 177-218, 176-218, 175-218, 174-218, 173-218, 172-218, 171-218, 170-218, 169-218, 168-218, 167-218, 166-218, 165-218, 164-218, 163-218, 162-218, 161-218, 160-218, 159-218, 158-218, 157-218, 156-218, 155-218, 154-218; 153-218; 152-218; 151-218; 150-218; 149-218; 148-218; 147-218; 146-218; 145-218; 144-218; 143-218; 142-218; 141-218; 140-218; 139-218; 138-218; 137-218; 136-218; 134-218; 133-218; 132-218; 131-218; 130-218; 129-218; 128-218; 127-218; 126-218; 125-218; 124-218; 123-218; 122-218; 121-218; 120-218; 119-218; 118-218; 117-218; 116-218; 115-218; 114-218; 113-218; 112-218; and 111-218. Furthermore, deletions of the amino acids at the positions corresponding to positions 215-218 of SEQ ID NO:26 may be combined with deletions at the positions corresponding to positions 165-182, such as 165-168, 165-169, 165-170, 165-171, 165-172, 165-173, 165-174, 165-175, 165-176, 165-177, 165-178, 165-179, 165-180, and 165-181. In various embodiments, the fragments of the amino acid sequence set forth in SEQ ID NO:26 include at least the amino acids corresponding to positions 11-19, 29-37, and/or 38-46 of SEQ ID NO:1, preferably 2 or all three of these amino acid stretches, since these represent the GG repeat sequences. In various embodiments, the fragments of SEQ ID NO:26 thus comprise at least amino acids 10-46, 10-50, 10-55, 1-46, 1-50, 1-55, 1-60, or 1-65, for example 1-39, 1-44, 1-47, or 1-49 of SEQ ID NO:26. In various embodiments, the first amino acid sequence is thus a fragment of SEQ ID NO:26 that has a C-terminal truncation, for example of amino acids in the positions corresponding to positions 215-218, 214-218, 185-218, 165-218, 135-218, or 111-218 of SEQ ID NO:26. In various embodiments the C-terminal end up to amino acid 110 (not including 110) in the numbering of SEQ ID NO:26 may be truncated. In various other embodiments, the amino acids in positions corresponding to positions 215-218 or 214-218 of SEQ ID NO:26 and additionally in the positions corresponding to positions 164-168, 165-168, 164-173, 165-173, 164-183, 165-183 or 165-182 of SEQ ID NO:26 may be deleted. In various embodiments any continuous 4 or more amino acids in the region corresponding to positions 164-183 of SEQ ID NO:26, in particular starting from position 164 or 165 and up to position 182 or 183 may be deleted. Exemplary embodiments of such truncations are set forth in SEQ ID Nos. 27-33. Further embodiments of such fragments of HlyA1 are set forth in SEQ ID Nos. 54-56. In various embodiments, such fragments of HlyA1 may, N-terminally to the sequences recited above, comprise any one of the amino acid sequences set forth in SEQ ID Nos. 21-24 and 102-105 and 107. These represent the GG repeat sequences or GG repeat-containing sequences or G-rich sequences of HlyA that are not included in the known fragment HlyA1 but are located N-terminal to the HlyA1 fragment in the native HlyA sequence. In various embodiments, the first amino acid sequence may comprise or consist of amino acids 10-46 of SEQ ID NO:26, or may, for example, comprise or consist of amino acids 10 to 44 or 1 to 46 or 1 to 39 of SEQ ID NO:26. In various embodiments, the first amino acid sequence consists of an amino acid sequence that starts with any one of amino acids 1, 2, 3, 4, 5, 6, 7, 8, 9 or 10 of SEQ ID NO:26 and includes up to amino acid 37, 38, 39, 40, 41, 42, 43, 45, 46, 47, 48, 49, 50, 51, 52, 53, 54, 55, 56, 57, 58, 59, 60, 61, 62, 63, 64, or 65 of SEQ ID NO:26. Exemplary fragments of HlyA1 (SEQ ID NO:26) that may be used in accordance with the present invention as first amino acid sequences include, without limitation, the amino acid sequences set forth in SEQ ID Nos. 34-56 and 117-155 as well as variants thereof, as herein defined. In various embodiments, the fragments of HlyA1 (SEQ ID NO:26) comprise the GG repeat(s) with the amino acid sequence set forth in SEQ ID NO:1, SEQ ID Nos. 1 and 2 or SEQ ID Nos. 1, 2 and 3. It is further preferred in various embodiments, that these fragments lack the C-terminal amino acids of SEQ ID NO:26, in particular the 4 C-terminal amino acids TTSA (SEQ ID NO:25). Even more preferred are those fragments that lack more than these 4 amino acid on the C-terminus, as defined above. Further fragments of HlyA are set forth in SEQ ID Nos. 102-105 and 283-287. These comprise GG repeat sequences set forth in SEQ ID Nos. 1-3 and 21, 22 and 24.

Examples of other fragments of RTX proteins are set forth in SEQ ID Nos. 57-62 and 65-105, 229-300 and 346-351. Fragments of RTX1 are set forth in SEQ ID Nos. 57-62 and 364. These comprise the GG repeat sequences set forth in SEQ ID Nos. 4, 5 and 63. Fragments of RTX7 are set forth in SEQ ID Nos. 65-69. These comprise GG repeat sequences set forth in SEQ ID Nos. 6-8. Fragments of RTX8 are set forth in SEQ ID Nos. 70-76. These comprise GG repeat sequences set forth in SEQ ID Nos. 9-12. Fragments of RTX24 are set forth in SEQ ID Nos. 77-80. These comprise GG repeat sequences set forth in SEQ ID Nos. 13-14. Fragments of RTX12 are set forth in SEQ ID Nos. 81-101 and 229-235. These comprise GG repeat sequences set forth in SEQ ID Nos. 15-20. Fragments of RTX18 are set forth in SEQ ID Nos. 236-241. Fragments of RTX22 are set forth in SEQ ID Nos. 242-246. Fragments of RTX30 are set forth in SEQ ID Nos. 247-252. Fragments of RTX33 are set forth in SEQ ID Nos. 253-256. Fragments of RTX28 are set forth in SEQ ID Nos. 257-261. Fragments of RTX24 are set forth in SEQ ID Nos. 262-269. Fragments of RTX19 are set forth in SEQ ID Nos. 270-274. Fragments of RTX16 are set forth in SEQ ID Nos. 275-278. Fragments of RTX26 are set forth in SEQ ID Nos. 279-282. Fragments of RTX3 are set forth in SEQ ID Nos. 288 and 289. Fragments of RTX20 are set forth in SEQ ID Nos. 290-291. Fragments of RTX31 are set forth in SEQ ID Nos. 292-293. Fragments of RTX14 are set forth in SEQ ID Nos. 294-295. Fragments of RTX32 are set forth in SEQ ID Nos. 296-298. Fragments of RTX29 are set forth in SEQ ID Nos. 299-300. Fragments of RTX 9 are set forth in SEQ ID Nos. 346-348 and 360. Fragments of RTX21 are set forth in SEQ ID Nos. 349-351 and 359. In various embodiments, the first amino acid sequence comprises, consists essentially or consist of any one of these non-HlyA RTX-derived sequences or is a variant thereof that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99% or 99.5% sequence identity with the respective amino acid sequence over its entire length, with the proviso that the GG consensus sequences (i.e. the invariable residues of GGxGxDxUx, but not the variable x and U residues) comprised therein are invariable. In various embodiments, the exact GG repeat sequences comprised therein are invariable.

All the afore-mentioned fragments may be used as first amino acid sequences in the sense of the present invention.

The fragments set forth in SEQ ID Nos. 57-62 and 65-105 and 229-282 and 288-300 and 346-351 and 359-360, 364-366, and 370-371 may be further truncated on their N- and/or C-terminal ends as long as at least one, two or more, preferably all, the GG repeats that are comprised in these fragments are retained. As disclosed above, it may be preferred that the sequences flanking the most N-terminal and most C-terminal GG repeat sequences are up to 60, up to 50, up to 40, up to 30 or up to 20 amino acids in length. In view of the yields of the fused peptide or protein, it is generally advantageous to make the first amino acid sequence as short as possible without significantly impairing its beneficial influence on expression, stability and/or renaturation. This means that any of the fragments set forth in SEQ ID Nos. 57-62 and 65-105 and 229-282 and 288-300 and 346-351 and 359-360, 364-366, and 370-371 may be N- and/or C-terminally truncated to have a maximum length of 190, 180, 170, 160, 150, 140, 130, 120, 110, 100, 90, 80, 70, or 60 amino acids or less.

The invention further comprises, as first amino acid sequences, variants of all the afore-mentioned fragments of RTX proteins. These variants may have over their entire length, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99% or 99.5% sequence identity with the respective amino acid sequence as set forth in SEQ ID Nos. 34-62, 65-105, 117-159, 229-300, 346-351, 359-360, 364-366, and 370-371, with the proviso that the GG consensus sequences (i.e. the invariable residues of GGxGxDxUx, but not the variable x and U residues) comprised therein are invariable. In various embodiments, the exact GG repeat sequences comprised therein are invariable. In various embodiments and as explained above, such variants may be truncated versions of the amino acid sequences as set forth in SEQ ID Nos. 34-62, 65-105, 117-159, 229-300, 346-351, 359-360, 364-366, and 370-371 as long as at least one, at least 2, at least 3, at least 4, at least 5, at least 6 or all GG repeat sequences comprised in the respective sequence are retained. In various embodiments, the variant is of the same length as the respective reference sequence. In other embodiments, it is a further shortened fragment thereof that may be obtainable by deletions/truncations. In various embodiments, the sequence of these variants with respect to the GG repeats and any linker sequences between the GG repeats is invariable, such that the variability is only given for the so-called flanking sequences that are N-terminal to the most N-terminal GG repeat and C-terminal to the most C-terminal GG repeat.

The first amino acid sequence of the present invention is, in various embodiments, 30 to 199 amino acids in length. The lower limit may be 35, 40, 45, 50, 55, 60, 65, 70, 75, 80, 85, 90, 95 or 100 amino acids in length. As disclosed above, it may be preferred that the first amino acid sequence is as short as possible, as long as its beneficial influence on expression levels and/or yields is not impaired and it retains sufficiently high solubility and/or proteolytic stability. Generally, the skilled person will know how to find a balance between a sequence that is as short as possible while still providing for the desired expression levels/yields within the scope defined by the present invention.

While the first amino acid sequence may correspond to a continuous amino acid stretch of any one of the amino acid sequences set forth in SEQ ID Nos. 34-62, 65-105, 117-159, 229-300, 346-351, 359-360, 364-366, and 370-371 having the indicated length, it is similarly possible that the first amino acid sequence corresponds to discontinuous stretches of the amino acid sequence set forth in these SEQ ID Nos, for example if it corresponds to stretches of these sequences with certain amino acids or amino acid sequences being deleted therefrom.

In various embodiments, if the first amino acid sequence is derived from SEQ ID NO:26, the first amino acid sequence does not comprise any one of the full amino acid sequences of SEQ ID Nos. 46-50 as set forth in WO 2013/057312 A1. In various other embodiments, the first amino acid sequence does not comprise any one of the full amino acid sequences of SEQ ID Nos. 33, 34, 36 of EP 2 583 975 A1, as well as SEQ ID Nos. 4 and 7 of WO 2006/036406 A2 and EPOP: A12703 (200 aa long C-terminal fragment of HlyA) of WO 8706953 A1. In various embodiments, the isolated polypeptide is not that of SEQ ID NO: 28, 30, 32, 34 or 36 of EP 2792686 A1. In various embodiments, the first amino acid is not derived from SEQ ID NO:26 and/or not derived from HlyA. In various embodiments, the first amino acid sequence is not the amino acid sequence set forth in SEQ ID NO.228. In various embodiments, the first amino acid sequence does not comprise the first amino acid sequence as disclosed in PCT/EP2021/056948 or EP20163961 or EP20168779, such as any one of SEQ ID Nos. 1, 8-26, 44-63, and 65-71 of PCT/EP2021/056948. In various embodiments, if the first amino acid sequence is derived from HlyA, in particular a fragment of the amino acid sequence set forth in SEQ ID NO:26, it does not comprise the amino acid sequences and/or C-terminal sequence set forth in any one SEQ ID Nos. 06, 325, 352-357, i.e. only covers two or all three GG repeat sequences and the GG-repeat-like sequence present in SEQ ID NO:26, and lacks the amino acids of 60-67, 60-218, 55-218, 50-218, 45-218 or 40-218, using the positional numbering of SEQ ID NO:28. It has been found that in certain embodiments, these sequences lower the renaturation performance of the first amino acid sequence, if present.

The first amino acid sequence may further comprise, in addition to the GG repeats, further glycine-rich motifs. These may be similar to the GG repeats but differ in one or more of the conserved amino acids. Exemplary glycine-rich motifs that may be included in the first amino acid sequence include, without limitation:

(SEQ ID NO: 301)
AGEGNDFAS,
(SEQ ID NO: 302)
AGYGRDVIT,
(SEQ ID NO: 303)
GAAGDDQVY,
(SEQ ID NO: 304)
GAGGADQLW,
(SEQ ID NO: 305)
GDAGNDLLF,
(SEQ ID NO: 306)
GDDGDDAVS,
(SEQ ID NO: 307)
GDAGDDFLE,
(SEQ ID NO: 308)
GDAGDDLLA,
(SEQ ID NO: 309)
GDEGDDQLF,
(SEQ ID NO: 310)
GEAGADQVF,
(SEQ ID NO: 311)
GEAGDDRLE,
(SEQ ID NO: 312)
GEAGNDTLS,
(SEQ ID NO: 313)
GEAGRDRLV,
(SEQ ID NO: 314)
GEAGSDILH,
(SEQ ID NO: 315)
GEDGNDFLV,
(SEQ ID NO: 316)
GEEGEDILL,
(SEQ ID NO: 317)
GEQGDDVLM,
(SEQ ID NO: 318)
GEQGEDVLL,
(SEQ ID NO: 319)
GEQGNDFVG,
(SEQ ID NO: 320)
GESGDDYVF,
(SEQ ID NO: 321)
GEWGNDFVS,
(SEQ ID NO: 322)
GFGGVDYLE,
(SEQ ID NO: 323)
GGGAADRMI,
(SEQ ID NO: 324)
GGIDRDVLT,
(SEQ ID NO: 326)
GGLAADVLL,
(SEQ ID NO: 327)
GGLGSDAFA,
(SEQ ID NO: 328)
GLAGNDVLH,
(SEQ ID NO: 329)
GLAGNDVLN,
(SEQ ID NO: 330)
GLDGNDHLI,
(SEQ ID NO: 331)
GLGGADVLN,
(SEQ ID NO: 332)
GNAGADALT,
(SEQ ID NO: 333)
GNAGDDLLK,
(SEQ ID NO: 334)
GNAGNDSLS,
(SEQ ID NO: 335)
GRAGNDTLI,
(SEQ ID NO: 336)
GRDGSDVLN,
(SEQ ID NO: 337)
GRGGNDTLI,
(SEQ ID NO: 338)
GSEGADLLD,
(SEQ ID NO: 339)
GTSGTDAIH,
(SEQ ID NO: 340)
GVSGSDTLI,
and
(SEQ ID NO: 341)
RGAGADTIT.

The glycine-rich, GG repeat-like motifs listed above may also be included as “Linkers”, as defined above, and thus serve to connect two GG repeat motifs. In some embodiments, the first amino acid sequence may not contain such GG repeat-like motifs or may not contain more than one or more than two of such sequences.

The isolated polypeptide comprises a second amino acid sequence N-terminal or C-terminal to the first amino acid sequence, wherein the second amino acid sequence is at least one peptide or polypeptide of interest. The second amino acid sequence may be 5 to 1500 amino acids in length, preferably 10, 12 or 15 to 1000, to 800, to 700, to 600, to 500, to 400, to 300, to 200, to 180, to 150, to 120 or to 100 amino acids in length. The terms “second amino acid sequence” and the terms “peptide of interest” and “polypeptide/protein of interest” are used interchangeably herein. “Peptides of interest” are typically oligopeptides with a length of up to 100 amino acids, typically 3 to 80 or 5 to 70 amino acids. “Polypeptides/proteins of interest” typically comprise more than 100 amino acids, with the upper limit as defined above. It has been found that some of the first amino acid sequences, such as those derived from for example RTX12, are particularly suited for facilitating the renaturation of peptides and polypeptides of interest, while other first amino acid sequences, such as those derived from RTX1, RTX16 and RTX18 also show good performance. In various embodiments, the GG repeat core sequence and fragments of RTX1, RTX12, RTX16 and RTX18 are preferred, with those of RTX12 being particularly preferred.

In various embodiments, the peptide or protein of interest may comprise two or more naturally occurring peptides or proteins, the two or more peptides or proteins may be separated by protease cleavage sites. This also includes embodiments, where the same peptide or protein is included multiple times in said second amino acid sequence. This then allows production of higher amounts of the respective peptide or protein of interest. In other embodiments, the peptide or protein of interest is only a single peptide or protein.

Generally, any peptide or protein may be chosen as protein of interest. In certain embodiments, the protein of interest is a protein, which does not form a homo-dimer or homo-multimer. The protein of interest may also be a peptide or protein which is a subunit of a larger peptide or protein complex. Such a peptide or protein may be isolated after expression and be suitable for an in vitro reconstitution of the multi peptide or protein complex. In certain embodiments, the protein or peptide of interest is a protein or peptide having less than 1000, less than 800, less than 700, less than 600, less than 500, less than 400, less than 300 amino acid residues, for example less than 200 amino acids or less than 150 amino acids. If these peptides comprise pre- and/or pro-sequences in their native state after translation the nucleic acid sequence encoding for the peptide of interest may be engineered to be limited to the sequence encoding the mature peptide. One exemplary peptide is insulin, e.g., human insulin. The expression of over-expressed peptides and proteins as inclusion bodies is especially advantageous where the peptide or protein is harmful to the host cell. For this reason, the present invention is particularly advantageous for expression of lipases and proteases which are known to be toxic to the host cell and thus the expression of these proteins by the inventive systems and methods represents a specific embodiment of the present invention.

In various embodiments, the peptide or protein of interest is an enzyme. The International Union of Biochemistry and Molecular Biology has developed a nomenclature for enzymes, the EC numbers; each enzyme is described by a sequence of four numbers preceded by “EC”. The first number broadly classifies the enzyme based on its mechanism. The complete nomenclature can be browsed at http://www.chem.qmul.ac.uk/iubmb/enzyme/.

Accordingly, a peptide or protein of interest according to the present invention may be chosen from any of the classes EC 1 (Oxidoreductases), EC 2 (Transferases), EC 3 (Hydrolases), EC 4 (Lyases), EC 5 (Isomerases), and EC 6 (Ligases), and the subclasses thereof.

In certain embodiments, the peptide or protein of interest is cofactor dependent or harbors a prosthetic group. For expression of such peptides or proteins, in some embodiments, the corresponding cofactor or prosthetic group may be added to the culture medium during expression.

In certain cases, the peptide or protein of interest is a dehydrogenase or an oxidase. In case the peptide or protein of interest is a dehydrogenase, in some embodiments, the peptide or protein of interest is chosen from the group consisting of alcohol dehydrogenases, glutamate dehydrogenases, lactate dehydrogenases, cellobiose dehydrogenases, formate dehydrogenases, and aldehydes dehydrogenases. In case the peptide or protein of interest is an oxidase, in some embodiments, the peptide or protein of interest is chosen from the group consisting of cytochrome P450 oxidoreductases, in particular P450 BM3 and mutants thereof, peroxidases, monooxygenases, hydrogenases, monoamine oxidases, aldehydes oxidases, xanthin oxidases, amino acid oxidases, and NADH oxidases.

In further embodiments, the peptide or protein of interest is a transaminase or a kinase. In case the peptide or protein of interest is a transaminase, in some embodiments, the peptide or protein of interest is chosen from the group consisting of alanine aminotransferases, aspartate aminotransferases, glutamate-oxaloacetic transaminases, histidinol-phosphate transaminases, and histidinol-pyruvate transaminases. In various embodiments, if the peptide or protein of interest is a kinase, the peptide or protein of interest is chosen from the group consisting of nucleoside diphosphate kinases, nucleoside monophosphate kinases, pyruvate kinase, and glucokinases. In some embodiments, if the peptide or protein of interest is a hydrolase, the peptide or protein of interest is chosen from the group consisting of lipases, amylases, proteases, cellulases, nitrile hydrolases, halogenases, phospholipases, and esterases.

In certain embodiments, if the peptide or protein of interest is a lyase, the peptide or protein of interest is chosen from the group consisting of aldolases, e.g., hydroxynitrile lyases, thiamine-dependent enzymes, e.g., benzaldehyde lyases, and pyruvate decarboxylases. In various embodiments, if the peptide or protein of interest is an isomerase, the peptide or protein of interest is chosen from the group consisting of isomerases and mutases.

In some embodiments, if the peptide or protein of interest is a ligase, the peptide or protein of interest may be a DNA ligase.

In certain embodiments, the peptide or protein of interest may be an antibody. This may include a complete immunoglobulin or fragment thereof, which immunoglobulins include the various classes and isotypes, such as IgA, IgD, IgE, IgGI, IgG2a, IgG2b and IgG3, IgM, etc. Fragments thereof may include Fab, Fv and F(ab′)2, Fab′, the variable domain of the light chain (VL) or the variable domain of the heavy chain (VH) and related fragments, such as nanobodies, and the like.

Also contemplated herein are therapeutically active peptides and proteins of interest, e.g., cytokines.

Thus, in certain embodiments the peptide or protein of interest is selected from the group consisting cytokines, in particular human or murine interferons, interleukins (IL-1, IL-2, IL-3, IL-4; IL-5; IL-6; IL-7; IL-8; IL-9; IL-10; IL-11; IL-12; IL-13; IL-14; IL-15; IL-16; and IL-17), colony-stimulating factors, necrosis factors, e.g., tumor necrosis factor, such as TNF alpha, and growth factors, such as transforming growth factor beta family members, such as TGF-beta1; TGF-beta2 and TGF-beta3.

In some embodiments, if the peptide or protein of interest is an interferon, the peptide or protein of interest may be selected from the group consisting of interferon alpha, e.g., alpha-1, alpha-2, alpha-2a, and alpha-2b, alpha-2, alpha-16, alpha 21, beta, e.g., beta-1, beta-1a, and beta-1b, or gamma.

In further embodiments, the peptide or protein of interest is an antimicrobial peptide, in particular a peptide selected from the group consisting of bacteriocines and lantibiotics, e.g., nisin, cathelicidins, defensins, and saposins.

Also disclosed herein are peptides or proteins of interest which are therapeutically active peptides or proteins. In certain embodiments, the peptide or protein of interest is a therapeutically active peptide. In some embodiments, a therapeutically active peptide may be selected from the group consisting of Fuzeon/T20, human calcitonin, salmon calcitonin, human corticotropin release factor, Mab40, Mab42, peptides associated with Alzheimer's disease, exenatide, Tesamorelin, Teriparatide, BMP-2, Corticorelin ovine triflutate, Linaclotide, Nesiritide, Lucinactant, Bivalirudin, Lepirudin, Thymalfasin, Glatiramer, Glucagon, Aviptadil, Secretin, Thymosin-b4, Teduglutide, GLP-1, GLP-2 and analoga, Plecanatide, Ecallantide, Anakinra, Disiteritide, Lixisenatide, Liraglutide, Semaglutide, Abaloparatide, Goserelin, Lanreotide, Carfilzomib, Enfuvirtide, T-20, Terlipressin, Elcatonin, Afamelanotide, Oxodotreotide, Caspofungin, Colistin, Polymyxin E, Cyclosporine, Dactinomcyin, Lyovac-Cosmegen, Degarelix, Vancomycin, Secretin, Ziconotide, Gonadorelin, Somastatin, Sincalide, Eptifibatid, Vapreotide, Triptorelin, Desmopressin, Lypressin, Atosiban, Pramintide, Pasireotide, Sandostatin, and Icatibant. The afore-mentioned peptides may be of mammalian or human origin. Also encompassed are analogues of the afore-mentioned peptides that originate from other species, for example homologues from other animals, microorganisms, virus and others.

In certain embodiments, the peptide or protein of interest is a type I secretion substrate. More than 1000 proteins are annotated or have been described as type I secretion substrates in the literature. Many of them have interesting characteristics for the biotechnological usage, in particular proteases and lipases. Suitable proteases and lipases have been described by Baumann et al. (1993) EMBO J 12, 3357-3364; and Meier et al. (2007) J. BIOL. CHEM.: 282(43), pp. 31477-31483. The content of each of these documents is incorporated by reference herein in its entirety.

In certain embodiments, the second amino acid sequence is a peptide or protein of interest which is chosen from the group consisting of MBP, lipase CalB, protease SprP, hydrolase PlaB, hydrolase Plak, hydrolase PlbF, lipase TesA, Vif, human interferon alpha-1, alpha-2, alpha-8, alpha-16, alpha-21, human interferon beta, human interferon gamma, murine interferon alpha, murine interferon gamma, IFABP, Cas2, affibody protein ZA3, nisin, corticotropin release factor, amyloid-beta peptide, exenatide, Fuzeon/T20, salmon calcitonin, Mab40, Mab42, lipase LipA, SprP, the HIV-1 protein Vif, human calcitonin, Tesamorelin, Teriparatide, BMP-2, Corticorelin ovine triflutate, Linaclotide, Nesiritide, Lucinactant, Bivalirudin, Lepirudin, Thymalfasin, Glatiramer, Glucagon, Aviptadil, Secretin, Thymosin-b4, Teduglutide, GLP-1, GLP-2 and analoga, Plecanatide, Ecallantide, Anakinra, Disiteritide, Lixisenatide, Liraglutide, Semaglutide, Abaloparatide, Goserelin, Lanreotide, Carfilzomib, Enfuvirtide, T-20, Terlipressin, Elcatonin, Afamelanotide, Oxodotreotide, Caspofungin, Colistin, Polymyxin E, Cyclosporine, Dactinomcyin, Lyovac-Cosmegen, Degarelix, Vancomycin, Secretin, Ziconotide, Gonadorelin, Somastatin, Sincalide, Eptifibatid, Vapreotide, Triptorelin, Desmopressin, Lypressin, Atosiban, Pramintide, Pasireotide, Sandostatin, and Icatibant.

The second amino acid sequence may be directly or via a linker sequence linked to the first amino acid sequence. It may be located N- or C-terminally relative to the first amino acid sequence, typically, for example but without limitation, C-terminally. This means that the N-terminus of the second amino acid sequence is linked, optionally via a linker sequence, to the C-terminus of the first amino acid sequence.

However, in other embodiments, the C-terminus of the second amino acid sequence may be fused, optionally via a linker sequence, to the N-terminus of the first amino acid sequence.

In various embodiments, the linker sequence that connects the first and second amino acid sequences is also a peptide sequence and connected to the respective ends of the first and second amino acid sequence via a peptide bond. In various embodiments, the linker sequence may be 1 to 50 amino acids in length, for example 1 to 30 amino acids or 5 to 20 amino acids. As disclosed above, the first amino acid sequence together with said linker sequence may have a maximum length of 250 amino acids. In various embodiments, the linker sequence is also heterologous to the first and the second amino acid sequence and/or does not naturally occur as part of the first and/or second amino acid sequence.

The linker sequence may be functional in that it may provide for easy cleavage and separation of the first and second amino acid. To facilitate such a purpose, it can comprise or consist of a protease recognition and cleavage site. The linker may also comprise both, a linker sequence that serves only as a link and a protease cleavage site. The linker sequence may be a G-rich sequence, for example 4 or 5 consecutive G residues, optionally followed by an S residue.

The term “protease (recognition and) cleavage site” refers to a peptide sequence which can be cleaved by a selected protease thus allowing the separation of peptide or protein sequences which are interconnected by a protease cleavage site. In certain embodiments the protease cleavage site is selected from the group consisting of a Factor Xa, a tobacco edge virus (TEV) protease, a enterokinase, a SUMO Express protease, an Arg-C proteinase, an Asp-N endopeptidases, an Asp-N endopeptidase+N-terminal Glu, a caspase 1, a caspase 2, a caspase 3, a caspase 4, a caspase 5, a caspase 6, a caspase 7, a caspase 8, a caspase 9, a caspase 10, a chymotrypsin-high specificity, a chymotrypsin-low specificity, a clostripain (Clostridiopeptidase B), a glutamyl endopeptidase, a granzyme B, a pepsin, a proline-endopeptidase, a proteinase K, Welqut protease, Clean Cut protease, a staphylococcal peptidase I, a Thrombin, a Trypsin, inteins, SprB or SplA-E from Staphylococcus aureus, and a Thermolysin cleavage site. In various embodiments, it may be a Factor Xa, SprB or TEV protease cleavage site. It can be preferred, in some embodiments, to design the protease recognition site such that as few amino acids as possible of the recognition and cleavage site remain attached to the peptide or protein of interest. In various embodiments, a protease recognition and cleavage site is included, the site being for example a TEV protease recognition and cleavage site. The TEV protease cleavage site typically comprises the amino acid sequence ENLYFQG/S (SEQ ID NO:64) and cleaves between the Gin (Q) and Gly/Ser (G/S) residues. In various embodiments, the P1′ amino acids may be A, M or C instead of G or S.

In various embodiments, the isolated polypeptide may further comprise at least one third amino acid sequence, for example an affinity tag.

The term “affinity tag” as used herein relates to entities which are coupled to a molecule of interest and allow enrichment of the complex between the molecule of interest and the affinity tag using an affinity tag receptor. In certain embodiments affinity tags may be selected from the group consisting of the Strep-tag® or Strep-tag® II, the myc-tag, the FLAG-tag, the His-tag, the small ubiquitin-like modifier (SUMO) tag, the covalent yet dissociable NorpD peptide (CYD) tag, the heavy chain of protein C (HPC) tag, the calmodulin binding peptide (CBP) tag, or the HA-tag or proteins such as Streptavidin binding protein (SBP), maltose binding protein (MBP), and glutathione-S-transferase.

In various embodiments, the isolated polypeptide has relative to a polypeptide comprising a first amino acid sequence not covered by the present definition, for example a first amino acid sequence as set forth in SEQ ID NO:26, an increased renaturation efficiency. “Increased”, as used in this connection, refers to yields of re-naturated polypeptides that are at least 10%, preferably at least 20% higher than those achieved with the reference sequence. Renaturation efficiency may be determined by comparing the levels of (i) renaturation of isolated polypeptides comprising as the first amino acid sequence a reference sequence and (ii) non-hydrolyzed isolated polypeptides comprising as the first amino acid sequence an amino acid sequence as described herein, after expression in form of inclusion bodies, purification and renaturation under otherwise identical conditions.

In various embodiments, the first amino acid sequence comprises as the first, N-terminal amino acid the residue M. If this is not present within the specific sequences disclosed herein, it may be artificially added, if desired, in particular to facilitate expression in a host cell.

The invention further relates to the nucleic acid, in particular the isolated nucleic acid molecule, encoding the polypeptide as described above. The polypeptide comprises the first amino acid sequence and the second amino acid sequence and optionally at least one third amino acid sequence. All of these amino acid sequences are typically linked by peptide bonds and expressed as a single fusion protein. To facilitate said expression, the nucleic acid molecule comprises a first nucleotide sequence encoding the first amino acid sequence and optionally a second, third and further nucleotide sequence encoding the second, third and further amino acid sequence, with said nucleotide sequences being operably linked to allow expression of the single fusion protein comprising all afore-mentioned amino acid sequences.

The term “operably linked” in the context of nucleic acid sequences means that a first nucleic acid sequence is linked to a second nucleic acid sequence such that the first nucleic acid sequence is placed in a functional relationship with the second nucleic acid sequence. For instance, a promoter sequence is operably linked to a coding sequence of a heterologous gene if the promoter can initiate the transcription of the coding sequence. In a further context, a sequence encoding for the first amino acid sequence is linked such to a second amino acid sequence encoding for a peptide or protein of interest, that if the two sequences are translated a single peptide/protein chain is obtained.

In certain embodiments, the above defined nucleic acid molecules may be comprised in a vector, for example a cloning or expression vector. Generally, the nucleic acid molecules of the invention can also be part of a vector or any other kind of cloning vehicle, including, but not limited to a plasmid, a phagemid, a phage, a baculovirus, a cosmid, or an artificial chromosome. Generally, a nucleic acid molecule disclosed in this application may be “operably linked” to a regulatory sequence (or regulatory sequences) to allow expression of this nucleic acid molecule.

Such cloning vehicles can include, besides the regulatory sequences described above and a nucleic acid sequence of the present invention, replication and control sequences derived from a species compatible with the host cell that is used for expression as well as selection markers conferring a selectable phenotype on transformed or transfected cells. Large numbers of suitable cloning vectors are known in the art, and are commercially available.

In certain embodiments the nucleic acid molecules disclosed herein are comprised in a cloning vector. In some embodiments the nucleic acid molecules disclosed herein are comprised in an expression vector. The vectors may comprise regulatory elements for replication and selection markers. In certain embodiments, the selection marker may be selected from the group consisting of genes conferring ampicillin, kanamycin, chloramphenicol, tetracycline, blasticidin, spectinomycin, gentamicin, hygromycin, and zeocin resistance. In various other embodiments, the selection may be carried out using antibiotic-free systems, for example by using toxin/antitoxin systems, cer sequence, triclosan, auxotrophies or the like. Suitable methods are known to those skilled in the art.

The above-described nucleic acid molecule of the present invention, comprising a nucleic acid sequence encoding for a protein of interest, if integrated in a vector, must be integrated such that the peptide or protein of interest can be expressed. Therefore, a vector of the present invention comprises sequence elements which contain information regarding to transcriptional and/or translational regulation, and such sequences are “operably linked” to the nucleotide sequence encoding the polypeptide. An operable linkage in this context is a linkage in which the regulatory sequence elements and the sequence to be expressed are connected in a way that enables gene expression. The precise nature of the regulatory regions necessary for gene expression may vary among species, but in general these regions comprise a promoter which, in prokaryotes, contains both the promoter per se, i.e. DNA elements directing the initiation of transcription, as well as DNA elements which, when transcribed into RNA, will signal the initiation of translation. Such promoter regions normally include 5′ non-coding sequences involved in initiation of transcription and translation, such as the −35/−10 boxes and the Shine-Dalgarno element in prokaryotes or the TATA box, CAAT sequences, and 5′-capping elements in eukaryotes. These regions can also include enhancer or repressor elements as well as translated signal and leader sequences for targeting the native polypeptide to a specific compartment of a host cell.

In addition, the 3′ non-coding sequences may contain regulatory elements involved in transcriptional termination, polyadenylation or the like. If, however, these termination sequences are not satisfactory functional in a particular host cell, then they may be substituted with signals functional in that cell.

In various embodiments, a vector comprising a nucleic acid molecule of the invention can therefore comprise a regulatory sequence, preferably a promoter sequence. In certain embodiments, the promoter is identical or homologous to promoter sequences of the host genome. In such cases endogenous polymerases may be capable to transcribe the nucleic acid molecule sequence comprised in the vector. In various embodiments, the promoter is selected from the group of weak, intermediate and strong promoters, preferably from weak to intermediate promoters.

In another preferred embodiment, a vector comprising a nucleic acid molecule of the present invention comprises a promoter sequence and a transcriptional termination sequence. Suitable promoters for prokaryotic expression are, for example, the araBAD promoter, the tet-promoter, the lacUV5 promoter, the CMV promo tor, the EF1 alpha promotor, the AOX1 promotor, the tac promotor, the T7promoter, or the lac promotor. Examples of promoters useful for expression in eukaryotic cells are the SV40 promoter or the CMV promoter. Furthermore, a nucleic acid molecule of the invention can comprise transcriptional regulatory elements, e.g., repressor elements, which allow regulated transcription and translation of coding sequences comprised in the nucleic acid molecule. Repressor element may be selected from the group consisting of the Lac-, AraC-, or MalR-repressor.

The vector may be effective for prokaryotic or eukaryotic protein expression. In particular, the nucleic acid molecules of the present invention may be comprised in a vector for prokaryotic protein expression. Such vector sequences are constructed such that a sequence of interest can easily be inserted using techniques well known to those skilled in the art. In certain embodiments, the vector is selected from the group consisting of a pET-vector, a pBAD-vector, a pK 184-vector, a pMONO-vector, a pSELECT-vector, pSELECT-Tag-vector, a pVITRO-vector, a pVIVO-vector, a pORF-vector, a pBLAST-vector, a pUO-vector, a pDUO-vector, a pZERO-vector, a pDeNy-vector, a pDRIVE-vector, a pDRIVE-SEAP-vector, a HaloTag®Fusion-vector, a pTARGET™-vector, a Flexi®-vector, a pDEST-vector, a pHIL-vector, a pPIC-vector, a pMET-vector, a pPink-vector, a pLP-vector, a pTOPO-vector, a pBud-vector, a pCEP-vector, a pCMV-vector, a pDisplay-vector, a pEF-vector, a pFL-vector, a pFRT-vector, a pFastBac-vector, a pGAPZ-vector, a plZ/V5-vector, a p3S-vector, a plAR-vector, pSEC, pMS, a pSU2726-vector, a pLenti6-vector, a pMIB-vector, a pOG-vector, a pOpti-vector, a pREP4-vector, a pRSET-vector, a pSCREEN-vector, a pSecTag-vector, a pTEFI-vector, a pTracer-vector, a pTrc-vector, a pUB6-vector, a pVAXI-vector, a pYC2-vector, a pYES2-vector, a pZeo-vector, a pcDNA-vector, a pFLAG-vector, a pTAC-vector, a pT7-vector, a Gateway®-vector, a pQE-vector, a pLEXY-vector, a pRNA-vector, a pPK-vector, a pUMVC-vector, a pLIVE-vector, a pCRUZ-vector, a Duet-vector, and other vectors or derivatives thereof.

The vectors of the present invention may be chosen from the group consisting of high, medium and low copy vectors.

The above described vectors of the present invention may be used for the transformation or transfection of a host cell in order to achieve expression of a peptide or protein which is encoded by an above described nucleic acid molecule and comprised in the vector DNA. Thus, in a further aspect, the present invention also relates to a host cell comprising a vector or nucleic acid molecule as disclosed herein.

Also contemplated herein are host cells, which comprise a nucleic acid molecule as described herein integrated into their genomes. The skilled person is aware of suitable methods for achieving the nucleic acid molecule integration. For example, the molecule may be delivered into the host cells by means of liposome transfer or viral infection and afterwards the nucleic acid molecule may be integrated into the host genome by means of homologous recombination. In certain embodiments, the nucleic acid molecule is integrated at a site in the host genome, which mediates transcription of the peptide or protein of the invention encoded by the nucleic acid molecule. In various embodiments, the nucleic acid molecule further comprises elements which mediate transcription of the nucleic acid molecule once the molecule is integrated into the host genome and/or which serve as selection markers.

In certain embodiments, the nucleic acid molecule of the present invention is transcribed by a polymerase natively encoded in the host genome. In various embodiments, the nucleic acid molecule is transcribed by a RNA-polymerase which is non-native to the host genome. In such embodiments, the nucleic acid molecule of the present invention may further comprise a sequence encoding for a polymerase and/or the host genome may be engineered or the host cell may be infected to comprise a nucleic acid sequence encoding for an exogenous polymerase. The host cell may be specifically chosen as a host cell capable of expressing the gene. In addition or otherwise, in order to produce the isolated polypeptide of the invention, the nucleic acid coding for it can be genetically engineered for expression in a suitable system. Transformation can be performed using standard techniques (Sambrook, J. et al. (2001), Molecular Cloning: A Laboratory Manual, 3^{rd} Ed., Cold Spring Harbor Laboratory Press, Cold Spring Harbor, NY).

Prokaryotic or eukaryotic host organisms comprising such a vector for recombinant expression of the polypeptide as described herein form also part of the present invention. Suitable host cells can be prokaryotic cells. In certain embodiments the host cells are selected from the group consisting of gram positive and gram negative bacteria. In some embodiments, the host cell is a gram negative bacterium, such as E. coli. In certain embodiments, the host cell is E. coli, in particular E. coli BL21 (DE3) or other E. coli K12 or E. coli B834 derivatives. In further embodiments, the host cell is selected from the group consisting of Escherichia coli (E. coli), Pseudomonas, Serratia marcescens, Salmonella, Shigella (and other enterobacteriaceae), Neisseria, Hemophilus, Klebsiella, Proteus, Enterobacter, Helicobacter, Acinetobacter, Moraxella, Helicobacter, Stenotrophomonas, Bdellovibrio, Legionella, acetic acid bacteria, Bacillus, Bacilli, Carynebacterium, Clostridium, Listeria, Streptococcus, Staphylococcus, and Archaea cells. Suitable eukaryotic host cells are among others CHO cells, insect cells, fungi, yeast cells, e.g., Saccharomyces cerevisiae, S. pombe, Pichia pastoris.

In certain embodiments, the host cell is a prokaryotic cell, such as E. coli, in particular E. coli BL21 (DE3), E. coli BL21, E. coli K12, E. coli BLR, E. coli BL21 AI, E. coli BL21 pLysS, E. coli XL1 and E. coli DH5a. Further suitable E. coli strains include, but are not limited to DH1, DH5a, DM1, HB101, JmIOI-110, Rosetta (DE3)pLysS, SURE, TOP10, XLI-Blue, XL2-Blue and XLIO-Blue strains.

The transformed host cells are cultured under conditions suitable for expression of the nucleotide sequence encoding the polypeptide of the invention. In certain embodiments, the cells are cultured under conditions suitable for expression of the nucleotide sequence encoding a polypeptide of the invention.

For producing the recombinant peptide or protein of interest in form of the fusion proteins described herein, a vector of the invention can be introduced into a suitable prokaryotic or eukaryotic host organism by means of recombinant DNA technology (as already outlined above). For this purpose, the host cell is first transformed with a vector comprising a nucleic acid molecule according to the present invention using established standard methods (Sambrook, J. et al. (2001), supra). The host cell is then cultured under conditions, which allow expression of the heterologous DNA and thus the synthesis of the corresponding polypeptide. Subsequently, the polypeptide is recovered (“isolated”) either from the cell or from the cultivation medium.

For expression of the peptides and proteins of the present invention several suitable protocols are known to the skilled person. The expression of a recombinant polypeptide of the present invention may be achieved by the following method comprising: (a) introducing a nucleic acid molecule or vector of the invention into a host cell, wherein the nucleic acid molecule or vector encodes the recombinant polypeptide; and (b) cultivating the host cell in a culture medium under conditions that allow expression of the recombinant polypeptide.

Step (a) may be carried out by using suitable transformation and transfection techniques known to those skilled in the art. These techniques are usually selected based on the type of host cell into which the nucleic acid is to be introduced. In some embodiments, the transformation may be achieved using electroporation or heat shock treatment of the host cell.

Step (b) may include a cultivation step that allows growth of the host cells. Alternatively, such step allowing growth of the host cells and a step that allows expression of the polypeptide may be performed separately in that the cells are first cultivated such that they grow to a desired density and then they are cultivated under conditions that allow expression of the polypeptide. The expression step can however still allow growth of the cells.

The method may further include a step of recovering the expressed polypeptide. The polypeptide may be recovered from the growth medium, if it is secreted, or from the cells or both. Preferably the polypeptide is recovered from the cells. The recovery of the polypeptide may include various purification steps.

Generally, any known culture medium suitable for growth of the selected host may be employed in this method. In various embodiments, the medium is a rich medium or a minimal medium. Also contemplated herein is a method, wherein the steps of growing the cells and expressing the peptide or protein comprise the use of different media. For example, the growth step may be performed using a rich medium which is replaced by a minimal medium in the expression step. In certain cases, the medium is selected from the group consisting of LB medium, TB medium, 2YT medium, synthetical medium and minimal medium.

In various preferred embodiments of the invention, the polypeptide of the invention is not secreted. In such embodiments, it may be expressed in form of inclusion bodies (IBs). In many cases, it may be useful to express the polypeptide of the invention in such an insoluble form, for example in cases where the peptide of interest is rather short, normally soluble and/or subject to proteolytic degradation within the host cell. Production of the peptide in insoluble form both facilitates simple recovery and additionally protects the peptide from the undesirable proteolytic degradation. In such embodiments, the first amino acid sequence may serve as a solubility tag, i.e., an inclusion body (IB) tag, that induces IB formation. Calcium ion (or earth alkaline metal ion) binding to the GG repeat(s) may later catalyze the folding of the fusion polypeptide into the native, active conformation with the calcium ions acting as a folding helper/chaperone. It has been described above, that the inventor has found a variety of first amino acid sequences that increase renaturation efficiency in such methods compared to the respective full length proteins they are derived from or longer fragments thereof. It has further surprisingly found that even comparably short first amino acid sequences can successfully induce proper renaturation of a comparably long second amino acid sequence, with said second amino acid sequence not comprising any such refolding sequence motifs, such as GG repeats.

The terms “inclusion body” or “IB”, as interchangeably used herein, relate to nuclear or cytoplasmic aggregates of substances, for instance proteins. Ibs are undissolved and have a non-unit lipid membrane. In the method of the present invention, the Ibs mainly consist of the fusion protein comprising at least one peptide/protein of interest and the first amino acid sequence, as defined herein.

In various embodiments, in particular where expression of the polypeptide of the invention in form of lbs is desired, the expression of the endogenous ABC transporter gene, the endogenous MFP gene and/or the endogenous OMP gene of the T1 SS or the activity of the corresponding gene products in the host cell is inhibited. In various embodiments, the host cell does not express endogenous ABC transporter, endogenous MFP and/or endogenous OMP of the T1 SS. The host cell may be engineered accordingly.

Methods to inhibit the expression of genes such as their deletion or insertion of nucleotide sequences destroying the integrity of the promoter sequence or the gene itself are known in the art. A preferred gene expression activity after deletion or disruption may be less than 35%, 30%, 25%, 20%, 15%, 10% or 5% of the activity measured in untreated cells. In other various embodiments of the invention, the endogenous ABC transporter, the endogenous MFP and/or the endogenous OMP of the type 1 secretion system are inhibited by antibodies or small molecule inhibitors. In preferred embodiments of the invention, the ABC transporter activity is inhibited by orthovanadate or an ATP homologous inhibitor such as 8-azido-ATP. Such ATP mimetics are known in the art. The preferred protein activity after inhibitor treatment may be less than 35%, 30%, 25%, 20%, 15%, 10% or 5% of the activity measured in untreated cells. In other embodiments of the invention, the transport is inhibited or blocked by the polypeptide of the invention itself, for example by over-expressing it.

In various embodiments of the afore-mentioned methods, the polypeptide of the invention is recovered from the host cells in form of insoluble inclusion bodies, for example by any of the purification methods disclosed herein.

Renaturation may then be effected by exposing the isolated/purified polypeptide to renaturation conditions, typically buffer conditions wherein the so-called refolding buffer comprises at least 0.01, more preferably 0.01-40 mM of alkaline earth metal ions, in particular Ca²⁺. By virtue of the presence of GG repeats in the first amino acid sequence, such treatment triggers refolding of the polypeptide into its functional (native) conformation. Interestingly, the refolding of the first amino acid sequence also induces proper folding of the peptide/polypeptide of interest fused thereto.

The general advantages of expression of a fusion protein in form of inclusion bodies are set forth in greater detail in WO 2014/170430 A1, which is herewith included by reference in its entirety.

In various embodiments, the method also encompasses the purification the recombinant polypeptide, wherein the recombinant polypeptide is purified using a method selected from affinity chromatography, ion exchange chromatography, reverse phase chromatography, size exclusion chromatography, and combinations thereof.

In several embodiments, the method may comprise the treatment of the recombinant polypeptide with a protease suitable for cleavage of a protease cleavage site within the recombinant polypeptide. In preferred embodiments, the recombinant polypeptide is purified and, optionally, renaturated prior to proteolytic cleavage using one or more methods disclosed above. Also after cleavage of the recombinant peptide or protein, the method may comprise a further purification step as defined above. Thus, in some embodiments the recombinant polypeptide is purified, subjected to proteolytic cleavage and the peptide or protein of interest is further purified. In other embodiments, the protease may be co-expressed or added to the cultivation medium or expressed by co-cultivated microorganisms, such that cleavage occurs before purification. However, since renaturation of the peptide or polypeptide of interest is mediated by the first amino acid sequence, such strategy may not be preferred.

In a further aspect, the present invention relates to the use of a vector or nucleic acid molecule as disclosed herein for the expression of a recombinant polypeptide. In some embodiments, the vector is used for the expression and optionally secretion of a recombinant polypeptide. The expression or expression and secretion may be achieved using the method described herein.

A method for expression of a recombinant peptide or protein using the above-described nucleic acid molecules may comprise the steps of:
(a) introducing a nucleic acid molecule or a vector as described above into a suitable host cell, wherein the nucleic acid molecule or vector encodes the recombinant polypeptide; and
(b) cultivating the host cell in a culture medium under conditions that allow expression of the recombinant polypeptide.

The expression of the recombinant polypeptide in step (b) may be in form of inclusion bodies.

The method can further be defined as the other methods of the invention described above. Specifically, the method may further comprise recovering the expressed peptide or protein from the host cell and/or the culture medium. In addition, the host cell may be a prokaryotic cell; and/or the expression may be performed in minimal culture medium; and/or the recombinant polypeptide may be purified using a method selected from affinity chromatography, ion exchange chromatography, reverse phase chromatography, size exclusion chromatography, and combinations thereof; and/or the method may comprises treatment of the recombinant polypeptide with a protease suitable for cleavage of a protease cleavage site within the recombinant polypeptide; and/or the method may comprise a cleavage step followed by purification of the recombinant polypeptide.

In various embodiments, the polypeptide is recovered in form of inclusion bodies and, after purification, exposed to a refolding buffer, wherein the refolding buffer comprises at least 0.01, more preferably 0.01-40 mM of earth alkaline metal ions, such as Ca²⁺. In various embodiments, the methods of the invention thus may also comprise a step of re-solubilizing the peptide/protein and/or reconstituting/refolding it under suitable conditions. Said step is also referred to herein as “renaturing” step.

In still another aspect, the present invention is therefore also directed to methods for renaturing the isolated polypeptide of the invention, wherein the method may comprise contacting, for example in form of re-solubilizing, the isolated polypeptide with a suitable medium, typically an aqueous medium comprising earth alkaline metal ions, for example the refolding buffer disclosed above. Such a medium allows binding of earth alkaline metal ions to the GG repeats (comprised in the first amino acid sequence) and induce conformational changes that in turn then facilitate refolding/renaturation of the fused heterologous peptide or polypeptide (second amino acid sequence).

In still another aspect, the present invention also relates to the use of an isolated polypeptide of the invention for facilitating the production of a recombinant peptide or protein. Said recombinant peptide of protein is part of the isolated polypeptide (in form of the second amino acid sequence), which is expressed as a fusion protein. In such embodiments, the first amino acid sequences disclosed herein may be used to facilitate renaturation/refolding of said recombinant peptide or polypeptide. Said recombinant peptide or protein is identical with the peptide or polypeptide of interest as referred to herein and may thus consist of the second amino acid sequence disclosed herein. As stated above, said use is preferably directed to recombinant polypeptides expressed as fusion proteins in a suitable expression system.

### EXAMPLES

### Materials and Methods

Expression host: Escherichia coli BL21 (DE3) (Novagen)

All oligonucleotides were purchased from Microsynth Seqlab GmbH.

Codon optimized nucleotide sequences encoding peptides were purchased from Thermo Fisher Scientific. All enzymes were purchased from NEB, Clontech, Invitrogen or Fermentas.

### Expression Protocol

1. Transformation of chemically competent cells with an expression vector encoding for a first amino acid sequence that is a GG repeat-containing fragment of a T1SS allocrite, as defined below, and, optionally, the peptide of interest (as a fusion protein) and plating of the transformed cells on LB agar plates comprising suitable antibiotic(s) for selection of the transformed cells.
2. Incubation of the agar plates over night at 37° C.
3. Inoculation of 2YT medium comprising antibiotics with a single colony from the agar plate for an overnight culture.
4. Incubation at 37° C. and shaking of the culture over night.
5. Inoculation of the main culture comprising 2×YT medium (16 g tryptone/peptone from casein (Roth, #8952.2), 10 g yeast extract (Roth, #2363.2), 5 g NaCl (Roth, #3957.1), ad 1 L demineralized water with the overnight culture resulting in an OD600 of 0.01-0.2 (flasks with baffles)
6. Incubation of the culture at 37° C. at different rpm
7. Induction of the expression the peptide or protein of interest with 1 mM IPTG at an OD600 of 0.4-1.0.
8. Incubation of the cultures for 3 hrs.
9. Culture samples were taken at 0 hrs and 3 hrs post induction and centrifuged for 10 min., 13,000×g, RT.
10. Cell samples were resuspended in water to adjust an OD of 5.0, mixed 4:1 with 5×SDS loading dye and heated (95° C., 10 min).
11. 20 μL samples were loaded on 15% SDS-PAGE gels and SDS-PAGE analysis was performed at 160 V for about 45 min.

### Example 1: Cloning of HlyA1 Fragments and Variants

The cloning of the various plasmids with HlyA1 truncations is based on the parental plasmid pSU-HlyA1 (SEQ ID NO: 108).

DNA fragments were amplified by Q5 High-Fidelity DNA Polymerase (according to the NEB protocol), digested with DpnI (according to the NEB protocol) and purified with SpeedBeads (DeAngelis M., Wang D. and Hawkins, T. 1995. Solid-phase reversible immobilization for the isolation of PCR products. Nucleic Acids Res. 23: 4742-4743.).

Plasmids were Created Using Two Cloning Strategies:
Cloning strategy 1: The amplification of a DNA fragment and linearization by phosphorylation and ligation (protocol according to NEB). Example: Primer 1 (SEQ ID NO:109) and Primer 2 (SEQ ID NO: 110) were used to generate construct 1.
Cloning strategy 2: The amplification of one or more DNA fragments with 15 bp complementary 5′ elongations which are linearized via Gibson reactions (Gibson, D. et al., 2009. Enzymatic assembly of DNA molecules up to several hundred kilobases. Nature Methods, 6: 343-5.). Example: Primer 3 (SEQ ID NO: 111) and Primer 4 (SEQ ID NO: 112) were used to amplify the plasmid backbone for construct 2. Primer 5 (SEQ ID NO:113) and primer 6 (SEQ ID NO:114) were used to amplify the DNA insert for construct 2.

### Example 2: Optimized HlyA1 Fragments for Renaturation

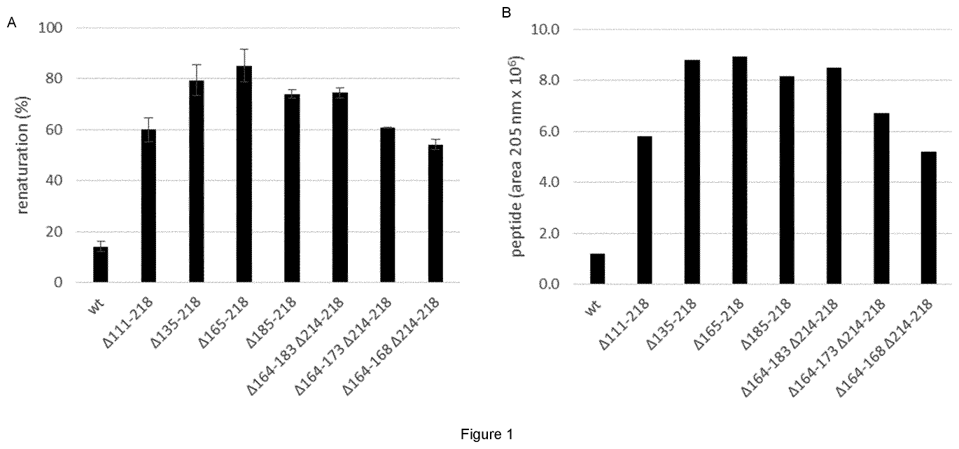

Fusion constructs of HlyA1 wt (SEQ ID NO:26) and deletion variants thereof (SEQ ID Nos. 27-33) as first amino acid sequence with a peptide of interest (SEQ ID NO: 115) C-terminally fused thereto via a linker comprising a 5 amino acid long G rich sequence (SEQ ID NO: 116) and a TEV protease site (SEQ ID NO:64) were designed. Fusion constructs were expressed as inclusion bodies (Ibs) in E. coli BL21 (DE3), Ibs were prepared via BugBuster Kit and the Ibs dissolved in 6 M GuHCl (1:4 w/v). After solubilization overnight, 1 mM stock solution of each construct was set in 6 M GuHCl. Renaturation was performed at room temperature for 20 min setting a final concentration of 0.05 mM in a total volume of 500 μL renaturation buffer (10 mM Tris-HCl, 120 mM NaCl, pH 7.3 supplemented with 20 mM CaCl₂)). The renaturation efficiency was determined in the cleared supernatant by UV/Vis spectroscopy (FIG. 1A). TEV-cleavage-reactions were performed for 3 h at 30° C. using the renatured protein sample and purified TEV-protease (1:25 molar ratio). The amount and identity of produced peptide was determined via HPLC/MS (FIG. 1B).

All truncated variants del111-218 (SEQ ID NO:22), del135-218 (SEQ ID NO:23), del165-218 (SEQ ID NO:24), del185-218 (SEQ ID NO:25), del164-183+del 214-218 (SEQ ID NO:26), del164-173+del 214-218 (SEQ ID NO:27), del164-168+del214-218 (SEQ ID NO:28), with the indicated SEQ ID Nos referring to the first amino acid sequence, show an increased renaturation efficiency compared to the HlyA1 wt (first amino acid: SEQ ID NO:26) construct (FIG. 1A). Almost quantitative TEV-cleavage occurred in all cases under the chosen reaction conditions. Therefore, the amount of peptide obtained (FIG. 1B) strictly depends on the renaturation efficiency (FIG. 1A).

The highest renaturation efficiency over all variants was observed for the variants Δ165-218 (first amino acid sequence: SEQ ID NO:29). The variants Δ135-218 (first amino acid sequence: SEQ ID NO:28), Δ185-218 (first amino acid sequence: SEQ ID NO:30) and Δ164-183 Δ214-218 (first amino acid sequence: SEQ ID NO:31) also show very high renaturation efficiencies. In all variants increased peptide yields due to better renaturation was observed.

Further HlyA1 fragments generated by deletion of amino acids 135-218 (SEQ ID NO:34), 130-218 (SEQ ID NO:35), 125-218 (SEQ ID NO:36), 120-218 (SEQ ID NO:37), 115-218 (SEQ ID NO:38), 110-218 (SEQ ID NO:39), 105-218 (SEQ ID NO:40), 95-218 (SEQ ID NO:42), 85-218 (SEQ ID NO:44), 45-218 (SEQ ID NO:52), and 40-218 (SEQ ID NO:53) and fused via a linker sequence (SEQ ID NO: 116) and a TEV protease site (SEQ ID NO:64) to a peptide of interest (67 amino acids long, pl 5.69; Mw 7.6 kDa). The results of the renaturation experiments are shown in Table 1 below.

TABLE 1

| | | Renaturation |
|---|---|---|
| | | efficiency (%) |
| Nr. | First amino acid sequence | NanoDrop-based |
| 1 | SEQ ID NO: 34 | 31.6 |
| 2 | SEQ ID NO: 35 | 38.4 |
| 3 | SEQ ID NO: 36 | 38.8 |
| 4 | SEQ ID NO: 37 | 32.6 |
| 5 | SEQ ID NO: 38 | 36.1 |
| 6 | SEQ ID NO: 39 | 24.6 |
| 7 | SEQ ID NO: 40 | 20.8 |
| 8 | SEQ ID NO: 42 | 22.3 |
| 9 | SEQ ID NO: 44 | 24.9 |
| 10 | SEQ ID NO: 52 | 26.2 |
| 11 | SEQ ID NO: 53 | 26.9 |
| 12 | SEQ ID NO: 24 | 54.3 |
| 13 | SEQ ID NO: 26 (control) | 25.8 |

It can be seen from the data in Table 1 that all truncated fusion constructs provided for similar or better renaturation efficiency compared to a longer control construct (first amino acid sequence SEQ ID NO:26). It needs to be noted that for short first amino acid sequence even renaturation efficiencies lower than the control may still be advantageous, as the proportion of the peptide/polypeptide of interest in the construct is significantly higher.

Further HlyA1 fragments generated by deletion of amino acids 120-218 (SEQ ID NO:155), 123-218 (SEQ ID NO:152), 127-218 (SEQ ID NO:148), 128-218 (SEQ ID NO:147), 129-218 (SEQ ID NO:146), 130-218 (SEQ ID NO:145), 131-218 (SEQ ID NO:144), 132-218 (SEQ ID NO:143), 135-218 (SEQ ID NO:140), 136-218 (SEQ ID NO:139), 137-218 (SEQ ID NO:138), 138-218 (SEQ ID NO:137), 139-218 (SEQ ID NO: 136), 140-218 (SEQ ID NO:135), 141-218 (SEQ ID NO:134), 142-218 (SEQ ID NO:133), 143-218 (SEQ ID NO:132), 144-218 (SEQ ID NO:131), 145-218 (SEQ ID NO:130), 146-218 (SEQ ID NO:129), 147-218 (SEQ ID NO: 128), 148-218 (SEQ ID NO:127), 149-218 (SEQ ID NO:126), 150-218 (SEQ ID NO:125), 151-218 (SEQ ID NO:124), 152-218 (SEQ ID NO:123), 153-218 (SEQ ID NO:122), 154-218 (SEQ ID NO:121), 155-218 (SEQ ID NO:120), 156-218 (SEQ ID NO:119), 157-218 (SEQ ID NO:118), and 158-218 (SEQ ID NO:117) and fused via a linker sequence (SEQ ID NO:116) and a TEV protease site (SEQ ID NO:64) to a peptide of interest (SEQ ID NO:115). The results of the renaturation experiments are shown in Table 2 below

TABLE 2

| Construct | Construct | | Renaturation | First amino |
|---|---|---|---|---|
| No. | size (kDa) | A260/280 | (%) | acid sequence |
| 1 | 20.5 | 0.55 | 83.7 | SEQ ID NO: 136 |
| 2 | 20.63 | 0.56 | 86.8 | SEQ ID NO: 135 |
| 3 | 20.7 | 0.56 | 87.2 | SEQ ID NO: 134 |
| 4 | 20.81 | 0.55 | 87.2 | SEQ ID NO: 133 |
| 5 | 20.94 | 0.54 | 88.3 | SEQ ID NO: 132 |
| 6 | 21.1 | 0.55 | 85.8 | SEQ ID NO: 131 |
| 7 | 21.23 | 0.57 | 85.7 | SEQ ID NO: 130 |
| 8 | 21.36 | 0.54 | 85.1 | SEQ ID NO: 129 |
| 9 | 21.52 | 0.55 | 88.6 | SEQ ID NO: 128 |
| 10 | 21.63 | 0.54 | 85.6 | SEQ ID NO: 127 |
| 11 | 21.74 | 0.54 | 87.6 | SEQ ID NO: 126 |
| 12 | 21.87 | 0.55 | 88.5 | SEQ ID NO: 125 |
| 13 | 21.94 | 0.55 | 87.2 | SEQ ID NO: 124 |
| 14 | 22.03 | 0.58 | 84.4 | SEQ ID NO: 123 |
| 15 | 22.19 | 0.56 | 82.2 | SEQ ID NO: 122 |
| 16 | 22.29 | 0.55 | 84.1 | SEQ ID NO: 121 |
| 17 | 22.46 | 0.54 | 76.6 | SEQ ID NO: 120 |
| 18 | 22.51 | 0.52 | 81.2 | SEQ ID NO: 119 |
| 19 | 22.63 | 0.53 | 83.0 | SEQ ID NO: 118 |
| 20 | 22.74 | 0.53 | 87.0 | SEQ ID NO: 117 |
| 21 | 18.33 | 0.54 | 67.6 | SEQ ID NO: 155 |
| 22 | 18.7 | 0.54 | 77.8 | SEQ ID NO: 152 |
| 23 | 19.2 | 0.55 | 83.1 | SEQ ID NO: 148 |
| 24 | 19.33 | 0.54 | 73.4 | SEQ ID NO: 147 |
| 25 | 19.42 | 0.55 | 78.2 | SEQ ID NO: 146 |
| 26 | 19.47 | 0.53 | 73.1 | SEQ ID NO: 145 |
| 27 | 19.63 | 0.56 | 73.4 | SEQ ID NO: 144 |
| 28 | 19.74 | 0.54 | 77.5 | SEQ ID NO: 143 |
| 29 | 20.06 | 0.60 | 87.4 | SEQ ID NO: 140 |
| 30 | 20.17 | 0.54 | 76.8 | SEQ ID NO: 139 |
| 31 | 20.26 | 0.53 | 77.7 | SEQ ID NO: 138 |
| 32 | 20.37 | 0.53 | 76.3 | SEQ ID NO: 137 |
| 33 (control) | | | 15.0 | SEQ ID NO: 26 |
| | | | | (control) |

Further constructs were made with another peptide of interest (SEQ ID NO:344). The HlyA fragments had the amino acid sequences set forth in SEQ ID Nos. 283 and 284. The results are set forth in Table 3.

TABLE 3

| | 1st amino acid | 2nd amino acid | Renaturation |
|---|---|---|---|
| Construct No. | sequence | sequence | (%) |
| P2102 | SEQ ID NO: 283 | SEQ ID NO: 344 | 26.9 |
| P2101 | SEQ ID NO: 284 | SEQ ID NO: 344 | 26.2 |
| Control | SEQ ID NO: 26 | SEQ ID NO: 344 | 25.0 |

These results show that the shorter fragments of HlyA comprising 2 or 3 GG repeats, namely the one with the amino acid sequences of SEQ ID NO:283 and 284 fully retain the renaturation ability of the much longer fragment of SEQ ID NO:26. It was found that fragments of SEQ ID NO:26 that comprise longer C-terminal extensions beyond the GG repeat region show significantly decreased renaturation performance comparted to the shorter ones limited to the GG repeat region. This demonstrates that the core GG repeat region is capable and sufficient, and in some embodiments even better suited (due to being shorter and thus beneficial for expression yields and/or having higher renaturation activity), for expressing, purifying and renaturing a peptide or polypeptide of interest.

### Example 3: RTX Protein Fragments for Renaturation

Fusion constructs of different RTX protein fragments (SEQ ID Nos: 57-59, 370 (RTX1); 65-66, 371 (RTX7), 70-71 (RTX8), 229-233 (RTX12), 236-240 (RTX18), 242-245 (RTX22), 247-250 (RTX30), 253-255 (RTX33), 257-260 (RTX28), 262-268 (RTX24), 270-273 (RTX19), 275-277 (RTX16), and 279-280 (RTX26)) as first amino acid sequences with a peptide of interest (SEQ ID NO:115) C-terminally fused thereto via a linker comprising a 5 amino acid long G rich sequence (SEQ ID NO:116) and a TEV protease site (SEQ ID NO:64) were designed. Fusion constructs were expressed as inclusion bodies (IBs) in E. coli BL21 (DE3), Ibs were prepared via BugBuster Kit and the Ibs dissolved in 6 M GuHCl (1:4 w/V). After solubilization overnight, 1 mM stock solution of each construct was set in 6 M GuHCl. Renaturation was performed at room temperature for 20 min setting a final concentration of 0.05 mM in a total volume of 500 μL renaturation buffer. The renaturation efficiency was determined in the cleared supernatant by UV/Vis spectroscopy as described in Example 2 above. In all constructs solubilization and renaturation was successfully achieved. The expression was rated as low (-), medium (*), good (**), very good (***) and excellent (****). Results are shown in Table 4.

TABLE 4

| | 1st amino acid | 2nd amino acid | Renaturation | |
|---|---|---|---|---|
| Construct | sequence | sequence | (%) | Expression |
| RTX12-P2111 | SEQ ID NO: 229 | SEQ ID NO: 115 | 65.3¹ | *** |
| RTX12-P2106 | SEQ ID NO: 230 | SEQ ID NO: 115 | 49.6¹ | * |
| RTX12-P2108 | SEQ ID NO: 231 | SEQ ID NO: 115 | 100.0¹ | *** |
| RTX12-P2109 | SEQ ID NO: 232 | SEQ ID NO: 115 | 92.2¹ | *** |
| RTX12-P2110 | SEQ ID NO: 233 | SEQ ID NO: 115 | 70.1¹ | **** |
| RTX12-P969 | SEQ ID NO: 342 | SEQ ID NO: 115 | 30.9¹ | |
| (control) | | | | |
| RTX18-P2141 | SEQ ID NO: 236 | SEQ ID NO: 115 | 101.9 | *** |
| RTX18-P2140 | SEQ ID NO: 237 | SEQ ID NO: 115 | 49.5 | *** |
| RTX18-P2142 | SEQ ID NO: 238 | SEQ ID NO: 115 | 38.1 | *** |
| RTX18-P2139 | SEQ ID NO: 239 | SEQ ID NO: 115 | 22.2 | *** |
| RTX18-P2143 | SEQ ID NO: 240 | SEQ ID NO: 115 | 21.0 | *** |
| RTX22-P2189 | SEQ ID NO: 242 | SEQ ID NO: 115 | 94.7 | nd |
| RTX22-P2197 | SEQ ID NO: 243 | SEQ ID NO: 115 | 78.3 | ** |
| RTX22-P2187 | SEQ ID NO: 244 | SEQ ID NO: 115 | 74.2 | nd |
| RTX22-P2188 | SEQ ID NO: 245 | SEQ ID NO: 115 | 84.7 | nd |
| RTX30-P2244 | SEQ ID NO: 247 | SEQ ID NO: 115 | 89.6 | nd |
| RTX30-P2241 | SEQ ID NO: 248 | SEQ ID NO: 115 | 19.0 | *** |
| RTX30-P2242 | SEQ ID NO: 249 | SEQ ID NO: 115 | 30.0 | *** |
| RTX30-P2243 | SEQ ID NO: 250 | SEQ ID NO: 115 | 30.6 | *** |
| RTX33-P2256 | SEQ ID NO: 253 | SEQ ID NO: 115 | 73.4 | nd |
| RTX33-P2257 | SEQ ID NO: 254 | SEQ ID NO: 115 | 100.8 | nd |
| RTX33-P2258 | SEQ ID NO: 255 | SEQ ID NO: 115 | 54.1 | nd |
| RTX28-P2233 | SEQ ID NO: 257 | SEQ ID NO: 115 | 57.3 | nd |
| RTX28-P2234 | SEQ ID NO: 258 | SEQ ID NO: 115 | 78.0 | * |
| RTX28-P2236 | SEQ ID NO: 259 | SEQ ID NO: 115 | 75.4 | * |
| RTX28-P2235 | SEQ ID NO: 260 | SEQ ID NO: 115 | 44.8 | * |
| RTX24-P2190 | SEQ ID NO: 262 | SEQ ID NO: 115 | 22.7 | **** |
| RTX24-P2191 | SEQ ID NO: 263 | SEQ ID NO: 115 | 25.6 | *** |
| RTX24-P2192 | SEQ ID NO: 264 | SEQ ID NO: 115 | 15.4 | *** |
| RTX24-P2193 | SEQ ID NO: 265 | SEQ ID NO: 115 | 19.9 | *** |
| RTX24-P2194 | SEQ ID NO: 266 | SEQ ID NO: 115 | 14.2 | *** |
| RTX24-P2195 | SEQ ID NO: 267 | SEQ ID NO: 115 | 13.9 | *** |
| RTX24-P2196 | SEQ ID NO: 268 | SEQ ID NO: 115 | 10.3 | *** |
| RTX19-P2145 | SEQ ID NO: 270 | SEQ ID NO: 115 | 76.5 | nd |
| RTX19-P2144 | SEQ ID NO: 271 | SEQ ID NO: 115 | 39.7 | nd |
| RTX19-P2146 | SEQ ID NO: 272 | SEQ ID NO: 115 | 29.9 | * |
| RTX19-P2147 | SEQ ID NO: 273 | SEQ ID NO: 115 | 20.0 | |
| RTX16-P2123 | SEQ ID NO: 275 | SEQ ID NO: 115 | 83.2 | *** |
| RTX16-P2124 | SEQ ID NO: 276 | SEQ ID NO: 115 | 96.3 | *** |
| RTX16-P2138 | SEQ ID NO: 277 | SEQ ID NO: 115 | 80.9 | *** |
| RTX26-P2230 | SEQ ID NO: 279 | SEQ ID NO: 115 | 72.5 | *** |
| RTX26-P2231 | SEQ ID NO: 280 | SEQ ID NO: 115 | 61.6 | nd |
| RTX26-P1826 | SEQ ID NO: 343 | SEQ ID NO: 115 | 30.4 | nd |
| (control) | | | | |
| RTX8-P2073 | SEQ ID NO: 70 | SEQ ID NO: 115 | 62.7 | * |
| RTX8-P2076 | SEQ ID NO: 71 | SEQ ID NO: 115 | 49.3 | * |
| RTX1-P2061 | SEQ ID NO: 57 | SEQ ID NO: 115 | 31.1 | *** |
| RTX1-P2062 | SEQ ID NO: 370 | SEQ ID NO: 115 | 59.5 | *** |
| RTX1-P2063 | SEQ ID NO: 58 | SEQ ID NO: 115 | 56.0 | *** |
| RTX1-P2065 | SEQ ID NO: 59 | SEQ ID NO: 115 | 52.0 | *** |
| RTX7-P2078 | SEQ ID NO: 371 | SEQ ID NO: 115 | 75.7 | nd |
| RTX7-P2080 | SEQ ID NO: 66 | SEQ ID NO: 115 | 50.2 | nd |
| RTX7-P2079 | SEQ IDNO: 65 | SEQ ID NO: 115 | 43.6 | nd |

nd = not determined
¹= normalized (to highest value above 100% (high value due to interference with slight precipitation))

Fusion constructs of different RTX protein fragments (SEQ ID Nos: 57-59, 71, 229-233, 236, 237, 239, 243, 272, 275-277, 350 and 351) as first amino acid sequences with a different polypeptide of interest (SEQ ID NO:345; antibody fragment) C-terminally fused thereto via a linker comprising a 5 amino acid long G rich sequence (SEQ ID NO:116) and a TEV protease site (SEQ ID NO:64) were designed. Fusion constructs were expressed as inclusion bodies and expression rated and renaturation determined as defined above. The results are shown in Table 5.

TABLE 5

| | 1st amino acid | 2nd amino acid | Renaturation | |
|---|---|---|---|---|
| Construct | sequence | sequence | (%) | Expression |
| RTX12-P2412 | SEQ ID NO: 229 | SEQ ID NO: 345 | 75.7¹ | *** |
| RTX12-P2408 | SEQ ID NO: 230 | SEQ ID NO: 345 | 82.0¹ | *** |
| RTX12-P2409 | SEQ ID NO: 231 | SEQ ID NO: 345 | 100.0¹ | *** |
| RTX12-P2410 | SEQ ID NO: 232 | SEQ ID NO: 345 | 80.5¹ | *** |
| RTX12-P2411 | SEQ ID NO: 233 | SEQ ID NO: 345 | 76.6¹ | *** |
| RTX18-P2418 | SEQ ID NO: 236 | SEQ ID NO: 345 | 77.7 | *** |
| RTX18-P2417 | SEQ ID NO: 237 | SEQ ID NO: 345 | 83.3 | *** |
| RTX18-P2416 | SEQ ID NO: 239 | SEQ ID NO: 345 | 10.8 | ** |
| RTX22-P2427 | SEQ ID NO: 243 | SEQ ID NO: 345 | 51.6 | ** |
| RTX16-P2123 | SEQ ID NO: 275 | SEQ ID NO: 345 | 54.6¹ | *** |
| RTX16-P2124 | SEQ ID NO: 276 | SEQ ID NO: 345 | 100.0¹ | *** |
| RTX16-P2138 | SEQ ID NO: 277 | SEQ ID NO: 345 | 36.0¹ | *** |
| RTX19-P2421 | SEQ ID NO: 272 | SEQ ID NO: 345 | 10.7 | *** |
| RTX8-P2407 | SEQ ID NO: 71 | SEQ ID NO: 345 | 19.4 | * |
| RTX21-P2426 | SEQ ID NO: 350 | SEQ ID NO: 345 | 89.9 | *** |
| RTX21-P2425 | SEQ ID NO: 351 | SEQ ID NO: 345 | 54.8 | *** |
| RTX1-P2400 | SEQ ID NO: 57 | SEQ ID NO: 345 | 25.8 | *** |
| RTX1-P2401 | SEQ ID NO: 58 | SEQ ID NO: 345 | 52.0 | *** |
| RTX1-P2402 | SEQ ID NO: 59 | SEQ ID NO: 345 | 45.1 | *** |

¹= normalized (to highest value above 100% (high value due to interference with slight precipitation))

Further designed were fragments of RTX12 (SEQ ID Nos 156, 157, 158 and 159) and C-terminally fused via a linker comprising a 5 amino acid long G rich sequence (SEQ ID NO: 116) and a TEV protease site (SEQ ID NO:64) to different peptides of interest (SEQ ID Nos. 160-164) in the following combinations: SEQ ID NO: 156+SEQ ID NO: 116+SEQ ID NO:64+SEQ ID NO: 160 (renaturation >90%); SEQ ID NO: 157+SEQ ID NO: 116+SEQ ID NO:64+SEQ ID NO: 161 (renaturation 78.9%); SEQ ID NO: 157+SEQ ID NO: 116+SEQ ID NO:64+SEQ ID NO: 162 (renaturation >90%); SEQ ID NO: 158+SEQ ID NO: 116+SEQ ID NO:64+SEQ ID NO: 163 (renaturation 75.7%); SEQ ID NO: 159+SEQ ID NO: 116+SEQ ID NO:64+a peptide of interest (67 amino acids long, pl 5.69; Mw 7.6 kDa) (renaturation >90%); SEQ ID NO: 158+SEQ ID NO: 116+SEQ ID NO:64+SEQ ID NO: 164 (renaturation >90%).

Fusion constructs of different RTX protein fragments (SEQ ID Nos: 57-59, 229-233, 236, 237, 239, 240, 243, 248-250, 258-260, 262-266, 275-277, 292, 350, 351, 365 and 366) as first amino acid sequences with two further peptides of interest (SEQ ID NO:361 und 362) C-terminally fused thereto via a linker comprising a 5 amino acid long G rich sequence (SEQ ID NO: 116) and a TEV protease site (SEQ ID NO:64) were designed. Fusion constructs were expressed as inclusion bodies and expression rated and renaturation determined as defined above. The results are shown in Table 6.

TABLE 6

| | 1st amino acid | 2nd amino acid | Renaturation | |
|---|---|---|---|---|
| Construct | sequence | sequence | (%) | Expression |
| RTX12-P2464 | SEQ ID NO: 230 | SEQ ID NO: 361 | 16.9¹ | ** |
| RTX12-P2465 | SEQ ID NO: 231 | SEQ ID NO: 361 | 50.0¹ | *** |
| RTX12-P2466 | SEQ ID NO: 232 | SEQ ID NO: 361 | 45.5¹ | *** |
| RTX12-P2467 | SEQ ID NO: 233 | SEQ ID NO: 361 | 100.0¹ | **** |
| RTX12-P2468 | SEQ ID NO: 229 | SEQ ID NO: 361 | 18.9¹ | ** |
| RTX12-P2526 | SEQ ID NO: 230 | SEQ ID NO: 362 | 47.0¹ | * |
| RTX12-P2527 | SEQ ID NO: 231 | SEQ ID NO: 362 | 50.0¹ | ** |
| RTX12-P2528 | SEQ ID NO: 232 | SEQ ID NO: 362 | 100.0¹ | ** |
| RTX12-P2529 | SEQ ID NO: 233 | SEQ ID NO: 362 | 45.0¹ | ** |
| RTX16-P2531 | SEQ ID NO: 275 | SEQ ID NO: 362 | 88.0 | ** |
| RTX16-P2532 | SEQ ID NO: 276 | SEQ ID NO: 362 | 100.0 | ** |
| RTX16-P2533 | SEQ ID NO: 277 | SEQ ID NO: 362 | 91.0 | ** |
| RTX18-P2472 | SEQ ID NO: 239 | SEQ ID NO: 361 | 90.0¹ | ** |
| RTX18-P2473 | SEQ ID NO: 237 | SEQ ID NO: 361 | 61.0¹ | ** |
| RTX18-P2474 | SEQ ID NO: 236 | SEQ ID NO: 361 | 70.0¹ | ** |
| RTX18-P2475 | SEQ ID NO: 238 | SEQ ID NO: 361 | 23.0¹ | ** |
| RTX18-P2476 | SEQ ID NO: 240 | SEQ ID NO: 361 | 100.0¹ | ** |
| RTX18-P2547 | SEQ ID NO: 239 | SEQ ID NO: 362 | 29.0 | * |
| RTX18-P2548 | SEQ ID NO: 237 | SEQ ID NO: 362 | 96.0 | * |
| RTX18-P2549 | SEQ ID NO: 236 | SEQ ID NO: 362 | 74.0 | * |
| RTX18-P2551 | SEQ ID NO: 240 | SEQ ID NO: 362 | 78.0 | * |
| RTX24-P2501 | SEQ ID NO: 262 | SEQ ID NO: 361 | 23.0¹ | **** |
| RTX24-P2502 | SEQ ID NO: 263 | SEQ ID NO: 361 | 13.0¹ | **** |
| RTX24-P2503 | SEQ ID NO: 264 | SEQ ID NO: 361 | 68.0¹ | *** |
| RTX24-P2504 | SEQ ID NO: 265 | SEQ ID NO: 361 | 54.0¹ | **** |
| RTX24-P2505 | SEQ ID NO: 266 | SEQ ID NO: 361 | 100.0¹ | *** |
| RTX22-P2508 | SEQ ID NO: 243 | SEQ ID NO: 361 | 100.0¹ | * |
| RTX28-P2511 | SEQ ID NO: 258 | SEQ ID NO: 361 | 48.0 | *** |
| RTX28-P2512 | SEQ ID NO: 259 | SEQ ID NO: 361 | 20.0 | *** |
| RTX28-P2513 | SEQ ID NO: 260 | SEQ ID NO: 361 | 34.0 | *** |
| RTX30-P2514 | SEQ ID NO: 248 | SEQ ID NO: 361 | 41.8¹ | *** |
| RTX30-P2515 | SEQ ID NO: 249 | SEQ ID NO: 361 | 62.7¹ | ** |
| RTX30-P2516 | SEQ ID NO: 250 | SEQ ID NO: 361 | 100.0¹ | *** |
| RTX31-P2517 | SEQ ID NO: 365 | SEQ ID NO: 361 | 100.0¹ | ** |
| RTX31-P2518 | SEQ ID NO: 292 | SEQ ID NO: 361 | 25.5¹ | ** |
| RTX32-P2521 | SEQ ID NO: 366 | SEQ ID NO: 361 | 100.0¹ | *** |
| RTX21-P2556 | SEQ ID NO: 350 | SEQ ID NO: 362 | 86.0 | * |
| RTX21-P2557 | SEQ ID NO: 351 | SEQ ID NO: 362 | 89.0 | * |
| RTX1-P2456 | SEQ ID NO: 57 | SEQ ID NO: 361 | 100.0¹ | *** |
| RTX1-P2457 | SEQ ID NO: 58 | SEQ ID NO: 361 | 51.8¹ | *** |
| RTX1-P2458 | SEQ ID NO: 59 | SEQ ID NO: 361 | 44.0¹ | *** |
| RTX1-P2495 | SEQ ID NO: 57 | SEQ ID NO: 362 | 100.0¹ | *** |
| RTX1-P2496 | SEQ ID NO: 58 | SEQ ID NO: 362 | 100.0¹ | *** |
| RTX1-P2497 | SEQ ID NO: 59 | SEQ ID NO: 362 | 87.6¹ | *** |

n.d. = not determined
¹= normalized (to highest value above 100% (high value due to interference with slight precipitation))

These results show that the renaturation efficiency is not dependent on the fused peptide or polypeptide but can be observed for a variety of different peptides/polypeptides of interest.

### Example 4: Artificial GG Repeat Peptides for Renaturation

A fusion construct of an artificial GG repeat sequence that comprises 3 RTX12 GG repeat core sequences (SEQ ID NO:235) directly linked by peptide bonds with an additional flanking sequence of 26 aa length on the N-terminus (SEQ ID NO:363) as first amino acid sequences with a peptide of interest (SEQ ID NO: 115) C-terminally fused thereto via a linker comprising a 5 amino acid long G rich sequence (SEQ ID NO: 116) and a TEV protease site (SEQ ID NO:64) was designed. The fusion construct was expressed as inclusion bodies (IBs) in E. coli BL21 (DE3), IBs were prepared via BugBuster Kit and the IBs dissolved in 6 M GuHCl (1:4 w/V). After solubilization overnight, 1 mM stock solution of the construct was set in 6 M GuHCl. Renaturation was performed at room temperature for 20 min setting a final concentration of 0.05 mM in a total volume of 500 μL renaturation buffer. The renaturation efficiency was determined in the cleared supernatant by UV/Vis spectroscopy as described in Examples 2 and 3 above. The renaturation efficiency was 78.4%, expression was very high, yielding tight inclusion bodies with the purity and quality of the renatured peptide being very high.

All documents cited herein, are hereby incorporated by reference in their entirety. The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group. Further embodiments of the invention will become apparent from the following claims.

1. An isolated polypeptide comprising a first and a second amino acid sequence, wherein:
(a) the first amino acid sequence is (a1) 30 to 200 amino acids in length, (a2) comprises at least one GG repeat sequence of the general consensus sequence GGxGxDxUx, wherein each x can independently be any amino acid and U is a hydrophobic, large amino acid selected from the group consisting of F, I, L, M, W, and Y, (a3) does not comprise a C-terminal secretion signal sequence and/or the C-terminal sequence TTSA (SEQ ID NO:21), and (a4) is not SEQ ID NO:228; and
(b) the second amino acid sequence is at least one peptide or polypeptide of interest, and
(c) wherein the first and second amino acid sequence are heterologous to each other.

2. The isolated polypeptide of claim 1, wherein the first amino acid sequence comprises at least 2, at least 3, at least 4, at least 5, or at least 6 GG repeat sequences of the general consensus sequence GGxGxDxUx.

3. The isolated polypeptide of claim 1, wherein the first amino acid sequence comprises a general structure, from N- to C-terminus, of:
(i) GGR1-Linker-GGR2; or
(ii) GGR1-Linker-GGR2-Linker-GGR3; or
(iii) GGR1-Linker-GGR2-Linker-GGR3-Linker-GGR4; or
(iv) GGR1-Linker-GGR2-Linker-GGR3-Linker-GGR4-Linker-GGR5; or
(v) GGR1-Linker-GGR2-Linker-GGR3-Linker-GGR4-Linker-GGR5-Linker-GGR6; or
(vi) GGR1-Linker-GGR2-Linker-GGR3-Linker-GGR4-Linker-GGR5-Linker-GGR6-Linker-GGR7; or
(vii) GGR1-Linker-GGR2-Linker-GGR3-Linker-GGR4-Linker-GGR5-Linker-GGR6-Linker-GGR7-Linker-GGR8;
wherein GGR1, GGR2, GGR3, GGR4, GGR5, GGR6, GGR7, and GGR8 are each independently a GG repeat of the consensus sequence GGxGxDxUx, wherein each x can independently be any amino acid and U is a hydrophobic, large amino acid selected from the group consisting of F, W, Y, I, L, and M, and wherein each “Linker” is independently a peptide bond or an amino acid sequence of 1 to 25 amino acids, 1 to 20 amino acids, or 1 to 15 amino acids in length.

4. The isolated polypeptide of claim 3, wherein the general structure is flanked by an additional N- and/or C-terminal amino acid sequence that may be 1 to 100 amino acids in length.

5. (canceled)

6. The isolated polypeptide of claim 1, wherein the consensus sequence of the GG repeat sequences is GGX¹GX²DX³UX⁴, wherein X¹ is selected from the group consisting of G, A, L, V, I, M, F, S, T, Q, Y, K, R, D, E, N, and Y; X² is selected from the group consisting of N, D, A, G, S, H, T, E, M, R, and H; X³ is selected from the group consisting of A, K, V, L, I, F, M, R, Y, S, L, T, V, Q, N, D, E, and H; and X⁴ is selected from the group consisting of W, K, R, Y, V, L, T, H, D, A, M, E, F, I, S, N, and Q.

7. The isolated polypeptide of claim 1, wherein the GG repeat sequence is selected from the group consisting of GGAGNDSYF (SEQ ID NO:8), GGAGNDIIY (SEQ ID NO:10), GGAGADTFV (SEQ ID NO:12), GGAGNDLME (SEQ ID NO:14), GGAGNDIIR (SEQ ID NO:18), GGAGDDTFV (SEQ ID NO:20), GGAGNDYLN (SEQ ID NO:23), GGAGADVLS (SEQ ID NO: 63), GGAGSDYLS (SEQ ID NO:165), GGAGADQLF (SEQ ID NO:166), GGAGDDTTV (SEQ ID NO:167), GGAGADDLT (SEQ ID NO:168), GGAGADNFI (SEQ ID NO:169), GGAGNDEVH (SEQ ID NO:170), GGAGNDYLS (SEQ ID NO:171), GGAGNDSLF (SEQ ID NO:172), GGAGFDILI (SEQ ID NO:173), GGAGNDVAF (SEQ ID NO:174), GGAGNNDIYH (SEQ ID NO:175), GGAGADSFV (SEQ ID NO:176), GGAGADALY (SEQ ID NO:177), GGAGSDAIV (SEQ ID NO:178), GGAGEDTFR (SEQ ID NO:179), GGAGHDRLA (SEQ ID NO:180), GGAGADTFV (SEQ ID NO:181), GGAGDDQLS (SEQ ID NO:182), GGAGDDVLE (SEQ ID NO:183), GGAGTDHLD (SEQ ID NO:184), GGAGNDRID (SEQ ID NO:185), GGAGADQLW (SEQ ID NO:186), GGAGNDTFV (SEQ ID NO:187), GGAGGDLLD (SEQ ID NO:188), GGAGEDSFR (SEQ ID NO:189), GGAGNDLME (SEQ ID NO:190), GGAGMDALH (SEQ ID NO:191), GGAGTDTLV (SEQ ID NO:192), GGAGADTLY (SEQ ID NO:193), GGAGADELT (SEQ ID NO:194), GGDGADRIS (SEQ ID NO:17), GGAGADRLD (SEQ ID NO:368), GGDGNDKLI (SEQ ID NO:22), GGDGDDELQ (SEQ ID NO:24), GGDGNDVLL (SEQ ID NO:195), GGDGNDSLV (SEQ ID NO:367), GGDGADLLF (SEQ ID NO:196), GGDGTDFLL (SEQ ID NO:197), GGEGDDLLK (SEQ ID NO:2), GGEGHDFVS (SEQ ID NO:198), GGEGDDRVY (SEQ ID NO:199), GGEGADLLF (SEQ ID NO:200), GGEGRDSLY (SEQ ID NO:227), GGEGNDHLR (SEQ ID NO:201), GGEGADRLI (SEQ ID NO:202), GGFGNDEVN (SEQ ID NO:203), GGGGDDIIV (SEQ ID NO:6), GGGGGDTLW (SEQ ID NO:11), GGGGHDRMQ (SEQ ID NO:19), GGGGSDIMR (SEQ ID NO:204), GGGGNDILI (SEQ ID NO:205), GGGGNDRLE (SEQ ID NO:206), GGGGSDMFV (SEQ ID NO:207), GGKGNDKLY (SEQ ID NO:1), GGKGDDYLE (SEQ ID NO:7), GGLGDDHLV (SEQ ID NO:15), GGLGSDVLD (SEQ ID NO:208), GGLGSDQLF (SEQ ID NO:209), GGLGADTLI (SEQ ID NO:210), GGLGSDAFA (SEQ ID NO:358), GGMGADELT (SEQ ID NO:211), GGNGDDQLY (SEQ ID NO:21), GGNGVDLAN (SEQ ID NO:369), GGQGNDVFV (SEQ ID NO:13), GGQGRDQLH (SEQ ID NO:212), GGRGSDLLI (SEQ ID NO:16), GGRGSDIFA (SEQ ID NO:213), GGRGSDLLD (SEQ ID NO:214), GGSGNDLLI (SEQ ID NO:9), GGSGNDRLI (SEQ ID NO:215), GGSGNDRLD (SEQ ID NO:216), GGSGNDDLS (SEQ ID NO:217), GGSGDDRYQ (SEQ ID NO:218), GGSGSDTFV (SEQ ID NO:219), GGTGNDRLW (SEQ ID NO:4), GGTGADIFV (SEQ ID NO:5), GGTGNDLVS (SEQ ID NO:220), GGTGGDTLS (SEQ ID NO:221), GGTGHDTLI (SEQ ID NO:222), GGTGSDRLV (SEQ ID NO:223), GGTGNDTYI (SEQ ID NO:224), GGTGRDVFL (SEQ ID NO:225), GGVGADTMT (SEQ ID NO:226), and GGYGNDIYR (SEQ ID NO:3).

8. The isolated polypeptide of claim 3, wherein
a) GGR1 is GGKGNDKLY (SEQ ID NO:1); GGR2 is GGEGDDLLK (SEQ ID NO:2); and GGR3 is GGYGNDIYR (SEQ ID NO:3);
b) GGR1 is GGTGNDRLW (SEQ ID NO:4); GGR2 is GGAGADVLS (SEQ ID NO:63); and GGR3 is GGTGADIFV (SEQ ID NO:5);
c) GGR1 is GGGGDDIIV (SEQ ID NO:6); GGR2 is GGKGDDYLE (SEQ ID NO:7); and GGR3 is GGAGNDSYF (SEQ ID NO:8);
d) GGR1 is GGSGNDLLI (SEQ ID NO:9); GGR2 is GGAGNDIIY (SEQ ID NO: 10); GGR3 is GGGGGDTLW (SEQ ID NO:11); and GGR4 is GGAGADTFV (SEQ ID NO:12);
e) GGR1 is GGQGNDVFV (SEQ ID NO:13) and GGR2 is GGAGNDLME (SEQ ID NO:14);
f) GGR1 is GGLGDDHLV (SEQ ID NO:15); GGR2 is GGRGSDLLI (SEQ ID NO:16); GGR3 is GGDGADRIS (SEQ ID NO:17); GGR4 is GGAGNDIIR (SEQ ID NO:18); GGR5 is GGGGHDRMQ (SEQ ID NO:19); and GGR6 is GGAGDDTFV (SEQ ID NO:20);
g) GGR1 is SEQ ID NO:21; GGR2 is SEQ ID NO:22; and GGR3 is SEQ ID NO: 24;
h) GGR1 is SEQ ID NO:221; GGR2 is SEQ ID NO: 195; GGR3 is SEQ ID NO: 168; GGR4 is SEQ ID NO:226; and GGR5 is SEQ ID NO: 169;
i) GGR1 is SEQ ID NO: 170; GGR2 is SEQ ID NO:227; GGR3 is SEQ ID NO: 208; GGR4 is SEQ ID NO: 171; GGR5 is SEQ ID NO:209; GGR6 is SEQ ID NO: 196; and GGR7 is SEQ ID NO:213; or
j) GGR1 is SEQ ID NO: 198; GGR2 is SEQ ID NO:203; GGR3 is SEQ ID NO: 199; GGR4 is SEQ ID NO:220; GGR5 is SEQ ID NO: 165; GGR6 is SEQ ID NO: 166; GGR7 is SEQ ID NO:200; and GGR8 is SEQ ID NO:358.

9. The isolated polypeptide of claim 8, wherein the first amino acid sequence comprises two or more sets of the GG repeat sequence combinations a)-i).

10. (canceled)

11. The isolated polypeptide of claim 1, wherein the polypeptide comprises at least two GG repeats and the at least two GG repeats are directly linked by a peptide bond.

12. The isolated polypeptide of claim 11, wherein the polypeptide comprises at least three GG repeats and the at least three GG repeats are directly linked to each other by a peptide bond.

13. The isolated polypeptide of claim 1, wherein the first amino acid sequence is 30 to 150 amino acids in length.

14. The isolated polypeptide of claim 1, wherein the first amino acid sequence comprises the amino acid sequence set forth in SEQ ID NO: 235, 241, 246, 251, 252, 256, 261, 269, 274, 278, 282, 289, 291, 293, 295, 297, 298, 300, 359, 360, or 364.

15. The isolated polypeptide of claim 1, wherein the first amino acid sequence has the amino acid sequence set forth in any one of SEQ ID NOs: 34-62, 65-105, 117-159, 229-300, 346-351, 359-360, 364-366, and 370-371, or a variant thereof, wherein said variant has at least 80% sequence identity with the respective reference amino acid sequence as set forth in SEQ ID NOs: 34-62, 65-105, 117-159, 229-300, 346-351, 359-360, 364-366, and 370-371, with the proviso that the GG consensus sequences comprised therein are invariable.

16. (canceled)

17. The isolated polypeptide of claim 1, wherein the second amino acid sequence is 10 to 1000 amino acids in length.

18. (canceled)

19. The isolated polypeptide of claim 1, wherein the second amino acid sequence is linked directly or via a linker sequence to the N- or C-terminal end of the first amino acid sequence, the linker sequence being 1 to 30 amino acids in length and comprising a protease recognition and cleavage site.

20-21. (canceled)

22. A nucleic acid encoding a polypeptide comprising a first and a second amino acid sequence, wherein:
(a) the first amino acid sequence is (a1) 30 to 200 amino acids in length, (a2) comprises at least one GG repeat sequence of the general consensus sequence GGxGxDxUx, wherein each x can independently be any amino acid and U is a hydrophobic, large amino acid selected from the group consisting of F, I, L, M, W, and Y, (a3) does not comprise a C-terminal secretion signal sequence and/or the C-terminal sequence TTSA (SEQ ID NO:21), and (a4) is not SEQ ID NO:228; and
(b) the second amino acid sequence is at least one peptide or polypeptide of interest, and
(c) wherein the first and second amino acid sequence are heterologous to each other.

23-26. (canceled)

27. A method for the production of a polypeptide of claim 1, comprising
(1) cultivating a host cell comprising a nucleic acid molecule encoding a polypeptide comprising a first and a second amino acid sequence under conditions that allow the expression of the polypeptide; wherein:
(a) the first amino acid sequence is (a1) 30 to 200 amino acids in length, (a2) comprises at least one GG repeat sequence of the general consensus sequence GGxGxDxUx, wherein each x can independently be any amino acid and U is a hydrophobic, large amino acid selected from the group consisting of F, I, L, M, W, and Y, (a3) does not comprise a C-terminal secretion signal sequence and/or the C-terminal sequence TTSA (SEQ ID NO:21), and (a4) is not SEQ ID NO:228;
(b) the second amino acid sequence is at least one peptide or polypeptide of interest, and
(c) wherein the first and second amino acid sequence are heterologous to each other;
(2) isolating the expressed polypeptide from the host cell; and
(3) renaturing the isolated polypeptide.

28. The method of claim 27, wherein the expressed polypeptide in the host cell is in the form of inclusion bodies.

29-30. (canceled)

31. The isolated polypeptide of claim 6, wherein X¹ is selected from the group consisting of G, A, E, S, T, Q, L, R, and D; X² is selected from the group consisting of N, D, A, S, and H; X³ is selected from the group consisting of T, R, V, L, S, I, A, Y, Q, D, and H; and X⁴ is selected from the group consisting of V, L, I, F, S, R, N, Y, and T.


SEQUENCE LISTING (371 sequences)

SEQ ID NO: 1 - protein, 9 aa, Artificial
  GG repeat
GGKGNDKLY

SEQ ID NO: 2 - protein, 9 aa, Artificial
  GG repeat
GGEGDDLLK

SEQ ID NO: 3 - protein, 9 aa, Artificial
  GG repeat
GGYGNDIYR

SEQ ID NO: 4 - protein, 9 aa, Artificial
  GG repeat
GGTGNDRLW

SEQ ID NO: 5 - protein, 9 aa, Artificial
  GG repeat
GGTGADIFV

SEQ ID NO: 6 - protein, 9 aa, Artificial
  GG repeat
GGGGDDIIV

SEQ ID NO: 7 - protein, 9 aa, Artificial
  GG repeat
GGKGDDYLE

SEQ ID NO: 8 - protein, 9 aa, Artificial
  GG repeat
GGAGNDSYF

SEQ ID NO: 9 - protein, 9 aa, Artificial
  GG repeat
GGSGNDLLI

SEQ ID NO: 10 - protein, 9 aa, Artificial
  GG repeat
GGAGNDIIY

SEQ ID NO: 11 - protein, 9 aa, Artificial
  GG repeat
GGGGGDTLW

SEQ ID NO: 12 - protein, 9 aa, Artificial
  GG repeat
GGAGADTFV

SEQ ID NO: 13 - protein, 9 aa, Artificial
  GG repeat
GGQGNDVFV

SEQ ID NO: 14 - protein, 9 aa, Artificial
  GG repeat
GGAGNDLME

SEQ ID NO: 15 - protein, 9 aa, Artificial
  GG repeat
GGLGDDHLV

SEQ ID NO: 16 - protein, 9 aa, Artificial
  GG repeat
GGRGSDLLI

SEQ ID NO: 17 - protein, 9 aa, Artificial
  GG repeat
GGDGADRIS

SEQ ID NO: 18 - protein, 9 aa, Artificial
  GG repeat
GGAGNDIIR

SEQ ID NO: 19 - protein, 9 aa, Artificial
  GG repeat
GGGGHDRMQ

SEQ ID NO: 20 - protein, 9 aa, Artificial
  GG repeat
GGAGDDTFV

SEQ ID NO: 21 - protein, 9 aa, Artificial
  GG repeat
GGNGDDQLY

SEQ ID NO: 22 - protein, 9 aa, Artificial
  GG repeat
GGDGNDKLI

SEQ ID NO: 23 - protein, 9 aa, Artificial
  GG repeat
GGAGNDYLN

SEQ ID NO: 24 - protein, 9 aa, Artificial
  GG repeat
GGDGDDELQ

SEQ ID NO: 25 - protein, 4 aa, Artificial
  Hly A C-terminal sequence
TTSA

SEQ ID NO: 26 - protein, 218 aa, Artificial
  Fragment of HlyA (HlyA1)
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKASYVYGNDALAYGSQGDLNPLINEISKIISAAGSFDVKEERTAASLLQLSGNASDFSYGRNSITLTTSA

SEQ ID NO: 27 - protein, 110 aa, Artificial
  HlyA1 fragment del111-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFE

SEQ ID NO: 28 - protein, 134 aa, Artificial
  HlyA1 fragment del135-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITP

SEQ ID NO: 29 - protein, 164 aa, Artificial
  HlyA1 fragment 165-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKASYVYGNDALAYGSQ

SEQ ID NO: 30 - protein, 184 aa, Artificial
  HlyA1 fragment 185-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKASYVYGNDALAYGSQGDLNPLINEISKIISAAGSF

SEQ ID NO: 31 - protein, 193 aa, Artificial
  HlyA1 fragment del164-183 del214-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKASYVYGNDALAYGSFDVKEERTAASLLQLSGNASDFSYGRNSIT

SEQ ID NO: 32 - protein, 203 aa, Artificial
  HlyA1 fragment del164-173 del214-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKASYVYGNDALAYGSISKIISAAGSFDVKEERTAASLLQLSGNASDFSYGRNSIT

SEQ ID NO: 33 - protein, 208 aa, Artificial
  HlyA1 fragment del164-168 del214-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKASYVYGNDALAYGSPLINEISKIISAAGSFDVKEERTAASLLQLSGNASDFSYGRNSIT

SEQ ID NO: 34 - protein, 134 aa, Artificial; identical to SEQ ID NO: 28
  HlyA1 truncation del135-218

SEQ ID NO: 35 - protein, 129 aa, Artificial
  HlyA1 truncation del130-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSG

SEQ ID NO: 36 - protein, 124 aa, Artificial
  HlyA1 truncation del125-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQI

SEQ ID NO: 37 - protein, 119 aa, Artificial
  HlyA1 truncation del120-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNH

SEQ ID NO: 38 - protein, 114 aa, Artificial
  HlyA1 truncation del115-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESG

SEQ ID NO: 39 - protein, 109 aa, Artificial
  HlyA1 truncation del110-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWF

SEQ ID NO: 40 - protein, 104 aa, Artificial
  HlyA1 truncation del105-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGIT

SEQ ID NO: 41 - protein, 99 aa, Artificial
  HlyA1 truncation del100-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGH

SEQ ID NO: 42 - protein, 94 aa, Artificial
  HlyA1 truncation del95-218
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNV

SEQ ID NO: 43 - protein, 89 aa, Artificial
  HlyA1 truncation del90-218
GNSLAKNVLFGGKGNDKLYGSEGADLLDGGEGDDLLKGGYGNDIYRYLSGYGHHIIDDDGGKEDKLSLADIDFRDVAFKREGNDLIMYK

SEQ ID NO: 44 - protein, 84 aa, Artificial
  HlyA1 truncation del85-218
GNSLAKNVLFGGKGNDKLYGSEGADLLDGGEGDDLLKGGYGNDIYRYLSGYGHHIIDDDGGKEDKLSLADIDFRDVAFKREGND

SEQ ID NO: 45 - protein, 79 aa, Artificial
  HlyA1 truncation del80-218
GNSLAKNVLFGGKGNDKLYGSEGADLLDGGEGDDLLKGGYGNDIYRYLSGYGHHIIDDDGGKEDKLSLADIDFRDVAFK

SEQ ID NO: 46 - protein, 74 aa, Artificial
  HlyA1 truncation del75-218
GNSLAKNVLFGGKGNDKLYGSEGADLLDGGEGDDLLKGGYGNDIYRYLSGYGHHIIDDDGGKEDKLSLADIDFR

SEQ ID NO: 47 - protein, 69 aa, Artificial
  HlyA1 truncation del70-218
GNSLAKNVLFGGKGNDKLYGSEGADLLDGGEGDDLLKGGYGNDIYRYLSGYGHHIIDDDGGKEDKLSLA

SEQ ID NO: 48 - protein, 64 aa, Artificial
  HlyA1 truncation del65-218
GNSLAKNVLFGGKGNDKLYGSEGADLLDGGEGDDLLKGGYGNDIYRYLSGYGHHIIDDDGGKED

SEQ ID NO: 49 - protein, 59 aa, Artificial
  HlyA1 truncation del60-218
GNSLAKNVLFGGKGNDKLYGSEGADLLDGGEGDDLLKGGYGNDIYRYLSGYGHHIIDDD

SEQ ID NO: 50 - protein, 54 aa, Artificial
  HlyA1 truncation del55-218
GNSLAKNVLFGGKGNDKLYGSEGADLLDGGEGDDLLKGGYGNDIYRYLSGYGHH

SEQ ID NO: 51 - protein, 49 aa, Artificial
  HlyA1 truncation del50-218
GNSLAKNVLFGGKGNDKLYGSEGADLLDGGEGDDLLKGGYGNDIYRYLS

SEQ ID NO: 52 - protein, 44 aa, Artificial
  HlyA1 truncation del 45-218
GNSLAKNVLFGGKGNDKLYGSEGADLLDGGEGDDLLKGGYGNDI

SEQ ID NO: 53 - protein, 39 aa, Artificial
  HlyA1 truncation del 40-218
GNSLAKNVLFGGKGNDKLYGSEGADLLDGGEGDDLLKGG

SEQ ID NO: 54 - protein, 26 aa, Artificial
  HlyA motif
GGKGNDKLYSEGADLLDGGEGDDLLK

SEQ ID NO: 55 - protein, 18 aa, Artificial
  HlyA motif
GGEGDDLLKGGYGNDIYR

SEQ ID NO: 56 - protein, 36 aa, Artificial
  HlyA motif
GGKGNDKLYGSEGADLLDGGEGDDLLKGGYGNDIYR

SEQ ID NO: 57 - protein, 170 aa, Artificial
  RTX1 fragment
NVKLDLVDPVASGGTTASEPAPTLQPATITGTSGANTLKGTTGSDVILGLGGADKLYGRAGADQLDGGTGNDRLWGGAGADVLSGGTGADIFVFDAPFVGAVDRITDFSPKDDVIYLQNSIFTGLRAAGAMSNAAFHAGTKAHDATDRVIYDGETGALYYDADGTGAMAA

SEQ ID NO: 58 - protein, 160 aa, Artificial
  RTX 1 fragment
NVKLDLVDPVASGGTTASEPAPTLQPATITGTSGANTLKGTTGSDVILGLGGADKLYGRAGADQLDGGTGNDRLWGGAGADVLSGGTGADIFVFDAPFVGAVDRITDFSPKDDVIYLQNSIFTGLRAAGAMSNAAFHAGTKAHDATDRVIYDGETGALYY

SEQ ID NO: 59 - protein, 150 aa, Artificial
  RTX1 fragment
NVKLDLVDPVASGGTTASEPAPTLQPATITGTSGANTLKGTTGSDVILGLGGADKLYGRAGADQLDGGTGNDRLWGGAGADVLSGGTGADIFVFDAPFVGAVDRITDFSPKDDVIYLQNSIFTGLRAAGAMSNAAFHAGTKAHDATDRVI

SEQ ID NO: 60 - protein, 27 aa, Artificial
  RTX1 GG repeat box
GGTGNDRLWGGAGADVLSGGTGADIFV

SEQ ID NO: 61 - protein, 18 aa, Artificial
  RTX1 GG repeats
GGTGNDRLWGGAGADVLS

SEQ ID NO: 62 - protein, 18 aa, Artificial
  RTX1 GG repeats
GGAGADVLSGGTGADIFV

SEQ ID NO: 63 - protein, 9 aa, Artificial
  GG repeat
GGAGADVLS

SEQ ID NO: 64 - protein, 7 aa, Artificial
  TEV protease site
  MISC_FEATURE (7)..(7): Xaa is G, S, A, M or C, preferably G or S
ENLYFQX

SEQ ID NO: 65 - protein, 127 aa, Artificial
  RTX7 fragment
DLYDQATGKGLSESWISDRAQMLSWMIQANTQDISTLPAITGDGLATIYSDWTSGKEVILRNDPIQSQHKVIFGRDSDQRDLIMGDNFSDRLYGGGGDDIIVGGKGDDYLEGGAGNDSYFINTGDGT

SEQ ID NO: 66 - protein, 136 aa, Artificial
  RTX7 fragment
DLYDQATGKGLSESWISDRAQMLSWMIQANTQDISTLPAITGDGLATIYSDWTSGKEVILRNDPIQSQHKVIFGRDSDQRDLIMGDNFSDRLYGGGGDDIIVGGKGDDYLEGGAGNDSYFINTGDGTDTIEDKQGD

SEQ ID NO: 67 - protein, 27 aa, Artificial
  RTX7 GG repeats
GGGGDDIIVGGKGDDYLEGGAGNDSYF

SEQ ID NO: 68 - protein, 18 aa, Artificial
  RTX7 GG repeats
GGGGDDIIVGGKGDDYLE

SEQ ID NO: 69 - protein, 18 aa, Artificial
  RTX7 GG repeats
GGKGDDYLEGGAGNDSYF

SEQ ID NO: 70 - protein, 120 aa, Artificial
  RTX8 fragment
QKINLNEGGFSDVGGLVGNVSIAHGVTVENAIGGSGNDLLIGNAAANILEGGAGNDIIYGGGGGDTLWGGAGADTFVYGAAADSTFDAPDWIMDFVSGEDKIDLTGIPQFASGGATLNFV

SEQ ID NO: 71 - protein, 130 aa, Artificial
  RTX8 fragment
QKINLNEGGFSDVGGLVGNVSIAHGVTVENAIGGSGNDLLIGNAAANILEGGAGNDIIYGGGGGDTLWGGAGADTFVYGAAADSTFDAPDWIMDFVSGEDKIDLTGIPQFASGGATLNFVSGFTGHAGDA

SEQ ID NO: 72 - protein, 45 aa, Artificial
  RTX8 GG repeat box
GGSGNDLLIGNAAANILEGGAGNDIIYGGGGGDTLWGGAGADTFV

SEQ ID NO: 73 - protein, 27 aa, Artificial
  RTX8 GG repeats
GGAGNDIIYGGGGGDTLWGGAGADTFV

SEQ ID NO: 74 - protein, 36 aa, Artificial
  RTX8 GG repeats
GGSGNDLLIGNAAANILEGGAGNDIIYGGGGGDTLW

SEQ ID NO: 75 - protein, 27 aa, Artificial
  RTX8 GG repeats
GGSGNDLLIGNAAANILEGGAGNDIIY

SEQ ID NO: 76 - protein, 18 aa, Artificial
  RTX8 GG repeats
GGGGGDTLWGGAGADTFV

SEQ ID NO: 77 - protein, 216 aa, Artificial
  RTX24 fragment
GGYNIILGGQGSNTLDLQQSVKNYSFASDGAGTLYLRDANGGISMTRDIGAIQSKEPGFLWGLFKDDVIHQVTDQGLKAGGQLTQYASSVRGDAGDNVLKAHAGGDWLFGLDGNDHLIGGQGNDVFVGGAGNDLMEAGGGNNTFLFTGHFGQDRILGYQGGDKLVFMGVSGVLPDQDYRAHASSSGNDTVLTFGQDSVTLVGVSLEHLNGSGIVLA

SEQ ID NO: 78 - protein, 137 aa, Artificial
  RTX24 fragment
GGQLTQYASSVRGDAGDNVLKAHAGGDWLFGLDGNDHLIGGQGNDVFVGGAGNDLMEAGGGNNTFLFTGHFGQDRILGYQGGDKLVFMGVSGVLPDQDYRAHASSSGNDTVLTFGQDSVTLVGVSLEHLNGSGIVLA

SEQ ID NO: 79 - protein, 47 aa, Artificial
  RTX24 GG repeat box
GGQGNDVFVGGAGNDLMEAGGGNNTFLFTGHFGQDRILGYQGGDKLV

SEQ ID NO: 80 - protein, 18 aa, Artificial
  RTX24 GG repeats
GGQGNDVFVGGAGNDLME

SEQ ID NO: 81 - protein, 140 aa, Artificial
  RTX12 fragment
IAATTKLGGGADEADYHTSLAAVKVDGGLGDDHLVGGRGSDLLIGGDGADRISGGAGNDIIRGGGGHDRMQGGAGDDTFVIGVGDLVTGDNQSDHIIDFRGAGSASGGEQDLLIFTGFSAGSKLVLDHDMGNTPTLQLYQ

SEQ ID NO: 82 - protein, 120 aa, Artificial
  RTX12 fragment
IAATTKLGGGADEADYHTSLAAVKVDGGLGDDHLVGGRGSDLLIGGDGADRISGGAGNDIIRGGGGHDRMQGGAGDDTFVIGVGDLVTGDNQSDHIIDFRGAGSASGGEQDLLIFTGFSA

SEQ ID NO: 83 - protein, 140 aa, Artificial
  RTX12 fragment
EQLPSAQVSSDHDASITRITIAATTKLGGGADEADYHTSLAAVKVDGGLGDDHLVGGRGSDLLIGGDGADRISGGAGNDIIRGGGGHDRMQGGAGDDTFVIGVGDLVTGDNQSDHIIDFRGAGSASGGEQDLLIFTGFSA

SEQ ID NO: 84 - protein, 150 aa, Artificial
  RTX12 fragment
EQLPSAQVSSDHDASITRITIAATTKLGGGADEADYHTSLAAVKVDGGLGDDHLVGGRGSDLLIGGDGADRISGGAGNDIIRGGGGHDRMQGGAGDDTFVIGVGDLVTGDNQSDHIIDFRGAGSASGGEQDLLIFTGFSAGSKLVLDHDM

SEQ ID NO: 85 - protein, 160 aa, Artificial
  RTX12 fragment
EQLPSAQVSSDHDASITRITIAATTKLGGGADEADYHTSLAAVKVDGGLGDDHLVGGRGSDLLIGGDGADRISGGAGNDIIRGGGGHDRMQGGAGDDTFVIGVGDLVTGDNQSDHIIDFRGAGSASGGEQDLLIFTGFSAGSKLVLDHDMGNTPTLQLYQ

SEQ ID NO: 86 - protein, 130 aa, Artificial
  RTX12 fragment
IAATTKLGGGADEADYHTSLAAVKVDGGLGDDHLVGGRGSDLLIGGDGADRISGGAGNDIIRGGGGHDRMQGGAGDDTFVIGVGDLVTGDNQSDHIIDFRGAGSASGGEQDLLIFTGFSAGSKLVLDHDM

SEQ ID NO: 87 - protein, 54 aa, Artificial
  RTX12 GG repeats
GGLGDDHLVGGRGSDLLIGGDGADRISGGAGNDIIRGGGGHDRMQGGAGDDTFV

SEQ ID NO: 88 - protein, 45 aa, Artificial
  RTX12 GG repeats
GGLGDDHLVGGRGSDLLIGGDGADRISGGAGNDIIRGGGGHDRMQ

SEQ ID NO: 89 - protein, 36 aa, Artificial
  RTX12 GG repeats
GGLGDDHLVGGRGSDLLIGGDGADRISGGAGNDIIR

SEQ ID NO: 90 - protein, 27 aa, Artificial
  RTX12 GG repeats
GGLGDDHLVGGRGSDLLIGGDGADRIS

SEQ ID NO: 91 - protein, 18 aa, Artificial
  RTX12 GG repeats
GGLGDDHLVGGRGSDLLI

SEQ ID NO: 92 - protein, 45 aa, Artificial
  RTX12 GG repeats
GGRGSDLLIGGDGADRISGGAGNDIIRGGGGHDRMQGGAGDDTFV

SEQ ID NO: 93 - protein, 36 aa, Artificial
  RTX12 GG repeats
GGDGADRISGGAGNDIIRGGGGHDRMQGGAGDDTFV

SEQ ID NO: 94 - protein, 27 aa, Artificial
  RTX12 GG repeats
GGAGNDIIRGGGGHDRMQGGAGDDTFV

SEQ ID NO: 95 - protein, 18 aa, Artificial
  RTX12 GG repeats
GGGGHDRMQGGAGDDTFV

SEQ ID NO: 96 - protein, 36 aa, Artificial
  RTX12 GG repeats
GGRGSDLLIGGDGADRISGGAGNDIIRGGGGHDRMQ

SEQ ID NO: 97 - protein, 27 aa, Artificial
  RTX12 GG repeats
GGRGSDLLIGGDGADRISGGAGNDIIR

SEQ ID NO: 98 - protein, 18 aa, Artificial
  RTX12 GG repeats
GGLGDDHLVGGRGSDLLI

SEQ ID NO: 99 - protein, 27 aa, Artificial
  RTX12 GG repeats
GGDGADRISGGAGNDIIRGGGGHDRMQ

SEQ ID NO: 100 - protein, 18 aa, Artificial
  RTX12 GG repeats
GGDGADRISGGAGNDIIR

SEQ ID NO: 101 - protein, 18 aa, Artificial
  RTX12 GG repeats
GGAGNDIIRGGGGHDRMQ

SEQ ID NO: 102 - protein, 36 aa, Artificial
  HlyA GG repeats
GGNGDDQLYGGDGNDKLIGGAGNNYLNGGDGDDELQ

SEQ ID NO: 103 - protein, 18 aa, Artificial
  HlyA GG repeats
GGNGDDQLYGGDGNDKLI

SEQ ID NO: 104 - protein, 27 aa, Artificial
  HlyA GG repeats
GGDGNDKLIGGAGNNYLNGGDGDDELQ

SEQ ID NO: 105 - protein, 84 aa, Artificial
  Hly GG repeats
GGNGDDQLYGGDGNDKLIGGAGNNYLNGGDGDDELQVQGNSLAKNVLFGGKGNDKLYGSEGADLLDGGEGDDLLKGGYGNDIYR

SEQ ID NO: 106 - protein, 8 aa, Artificial
  G rich sequence motif
GGKEDKLS

SEQ ID NO: 107 - protein, 9 aa, Artificial
  G rich sequence motif
GGAGNNYLN

SEQ ID NO: 108 - DNA, 4545 bp, Artificial
  pSU HlyA1 vector
tgattacgaattcgagctcccaaaactcatgttggtaaagtatcagaatttcatggaggtaaaattgataaacagttagcgaataaaatttttaaacaatatcaccacgagttaataactgaagtaaaaagaaagtcagattttaatttttcattaactggttaagaggtaattaaatgggaaattctcttgcaaaaaatgtattattcggtggaaaaggtaatgacaagctgtacggcagtgagggggcagatctgcttgatggtggagagggggatgatctcctgaaaggcggatatggtaatgatatttatcgttatctgtcaggatatggtcatcatattattgatgatgatggggggaaagaggataaactcagtttggctgatattgatttccgggatgtggccttcaagcgagaaggtaatgacctcatcatgtataaagctgaaggtaatgttctttccattggtcataaaaatggtattacattcaggaactggtttgaaaaagagtcaggtgatatctctaatcaccagatagagcagatttttgataaaagtggccggataatcacacctgattcccttaaaaaggcacttgagtatcaacagcgtaataataaggcaagttatgtgtatgggaatgatgcattagcctatggaagtcagggtgatcttaatccattaattaatgaaatcagcaaaatcatttcagctgcaggtagcttcgatgttaaagaggaaagaactgcagcttctttattgcagttgtccggtaatgccagtgatttttcatatggacggaactcaataaccctgaccacatcagcataatatattaatttaaatgatagcaatcttactgggctgtgccacataagattgctattttttttggagtcataatggattcttgtcataaaattgattatgggttatacgccctggagattttagcccaataccataacgtctctgttaacccggaagaaattaaacatagatttgatacagacgggacaggtctgggattaacgtcatggttgcttgctgcgaaatctttagaactaaaggtaaaacaggtaaaaaaaacaattgatcgatgataagctgtcaaacatgagaattacaacttatatcgtatggggctgacttcaggtgctacatttgaagagataaattgcactgaaatcctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgccgttaacggattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgccgttaacggccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaca

SEQ ID NO: 109 - DNA, 41 bp, Artificial
  oligonucleotide primer
catttaattacctcttaaccagttaatgaaaaattaaaatc

SEQ ID NO: 110 - DNA, 21 bp, Artificial
  oligonucleotide primer
aggaactggtttgaaaaagag

SEQ ID NO: 111 - DNA, 33 bp, Artificial
  oligonucleotide primer
caaactgagtttatcatcaagcagatctgcccc

SEQ ID NO: 112 - DNA, 22 bp, Artificial
  oligonucleotide primer
ttagcctatggaagtcagggtg

SEQ ID NO: 113 - DNA, 34 bp, Artificial
  oligonucleotide primer
gataaactcagtttggctgatattgatttccggg

SEQ ID NO: 114 - DNA, 34 bp, Artificial
  oligonucleotide primer
acttccataggctaatttaagggaatcaggtgtg

SEQ ID NO: 115 - protein, 34 aa, Artificial
  peptide of interest
SVSEIQLMHNLGKHLNSMERVEWLRKKLQDVHNF

SEQ ID NO: 116 - protein, 5 aa, Artificial
  linker sequence
GGGGS

SEQ ID NO: 117 - protein, 157 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKASYVYGND

SEQ ID NO: 118 - protein, 156 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKASYVYGN

SEQ ID NO: 119 - protein, 155 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKASYVYG

SEQ ID NO: 120 - protein, 154 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKASYVY

SEQ ID NO: 121 - protein, 153 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKASYV

SEQ ID NO: 122 - protein, 152 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKASY

SEQ ID NO: 123 - protein, 151 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKAS

SEQ ID NO: 124 - protein, 150 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNKA

SEQ ID NO: 125 - protein, 149 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNNK

SEQ ID NO: 126 - protein, 148 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRNN

SEQ ID NO: 127 - protein, 147 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQRN

SEQ ID NO: 128 - protein, 146 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQR

SEQ ID NO: 129 - protein, 145 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQQ

SEQ ID NO: 130 - protein, 144 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEYQ

SEQ ID NO: 131 - protein, 143 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALEY

SEQ ID NO: 132 - protein, 142 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKALE

SEQ ID NO: 133 - protein, 141 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKAL

SEQ ID NO: 134 - protein, 140 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKKA

SEQ ID NO: 135 - protein, 139 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLKK

SEQ ID NO: 136 - protein, 138 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSLK

SEQ ID NO: 137 - protein, 137 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDSL

SEQ ID NO: 138 - protein, 136 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPDS

SEQ ID NO: 139 - protein, 135 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIITPD

SEQ ID NO: 140 - protein, 134 aa, Artificial; identical to SEQ ID NO: 28
  HlyA fragment

SEQ ID NO: 141 - protein, 133 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRIIT

SEQ ID NO: 142 - protein, 132 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRII

SEQ ID NO: 143 - protein, 131 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGRI

SEQ ID NO: 144 - protein, 130 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKSGR

SEQ ID NO: 145 - protein, 129 aa, Artificial; identical to SEQ ID NO: 35
  HlyA fragment

SEQ ID NO: 146 - protein, 128 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDKS

SEQ ID NO: 147 - protein, 127 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFDK

SEQ ID NO: 148 - protein, 126 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIFD

SEQ ID NO: 149 - protein, 125 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQIF

SEQ ID NO: 150 - protein, 124 aa, Artificial; identical to SEQ ID NO: 36
  HlyA fragment

SEQ ID NO: 151 - protein, 123 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIEQ

SEQ ID NO: 152 - protein, 122 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQIE

SEQ ID NO: 153 - protein, 121 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQI

SEQ ID NO: 154 - protein, 120 aa, Artificial
  HlyA fragment
GNSLAKNVLFGGKGNDKLYGSEGADLLDGGEGDDLLKGGYGNDIYRYLSGYGHHIIDDDGGKEDKLSLADIDFRDVAFKREGNDLIMYKAEGNVLSIGHKNGITFRNWFEKESGDISNHQ

SEQ ID NO: 155 - protein, 119 aa, Artificial; identical to SEQ ID NO: 37
  HlyA fragment

SEQ ID NO: 156 - protein, 175 aa, Artificial
  RTX12 fragment
EQLPSAQVSSDHDASITRITIAATTKLGGGADEADYHTSLAAVKVDGGLGDDHLVGGRGSDLLIGGDGADRISGGAGNDIIRGGGGHDRMQGGAGDDTFVIGVGDLVTGDNQSDHIIDFRGAGSASGGEQDLLIFTGFSAGSKLVLDHDMGNTPTLQLYQVYNGASLVGSLLIQT

SEQ ID NO: 157 - protein, 190 aa, Artificial
  RTX12 fragment
EQLPSAQVSSDHDASITRITIAATTKLGGGADEADYHTSLAAVKVDGGLGDDHLVGGRGSDLLIGGDGADRISGGAGNDIIRGGGGHDRMQGGAGDDTFVIGVGDLVTGDNQSDHIIDFRGAGSASGGEQDLLIFTGFSAGSKLVLDHDMGNTPTLQLYQVYNGASLVGSLLIQTNTAAHLTSGDYLFQA

SEQ ID NO: 158 - protein, 115 aa, Artificial
  RTX12 fragment
EQLPSAQVSSDHDASITRITIAATTKLGGGADEADYHTSLAAVKVDGGLGDDHLVGGRGSDLLIGGDGADRISGGAGNDIIRGGGGHDRMQGGAGDDTFVIGVGDLVTGDNQSDH

SEQ ID NO: 159 - protein, 145 aa, Artificial
  RTX12 fragment
EQLPSAQVSSDHDASITRITIAATTKLGGGADEADYHTSLAAVKVDGGLGDDHLVGGRGSDLLIGGDGADRISGGAGNDIIRGGGGHDRMQGGAGDDTFVIGVGDLVTGDNQSDHIIDFRGAGSASGGEQDLLIFTGFSAGSKLV

SEQ ID NO: 160 - protein, 175 aa, Artificial
  peptide of interest AG_L2
PLLPFYPPDEALEIGLELNSSALPPTEGGGGSGGGGSVILPQAPSGPSYATYLQPAQAQMLTPPGGGGSGGGGSDKPLRRNNSYTSYIMAICGMPLDSFRAGGGGSGGGGSEVIQTSKYYMRDVIAIESAWLLELAPHGGGGSGGGGSEHIHRAGGLFVADAIQVGFGRIGKHFW

SEQ ID NO: 161 - protein, 115 aa, Artificial
  peptide of interest BMP-2
MQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 162 - protein, 86 aa, Artificial
  peptide of interest glargine
FVNQHLCGSHLVEALYLVCGERGFFYTPKTRREAEDLQVGQVELGGGPGAGSLQPLALEGSLQARGIVEQCCTSICSLYQLENYCG

SEQ ID NO: 163 - protein, 191 aa, Artificial [sequence omitted]

SEQ ID NO: 164 - protein, 86 aa, Artificial
  peptide of interest LisPro
FVNQHLCGSHLVEALYLVCGERGFFYTKPTRREAEDLQVGQVELGGGPGAGSLQPLALEGSLQARGIVEQCCTSICSLYQLENYCN

SEQ ID NO: 165 - protein, 9 aa, Artificial
  GG repeat
GGAGSDYLS

SEQ ID NO: 166 - protein, 9 aa, Artificial
  GG repeat
GGAGADQLF

SEQ ID NO: 167 - protein, 9 aa, Artificial
  GG repeat
GGAGDDTTV

SEQ ID NO: 168 - protein, 9 aa, Artificial
  GG repeat
GGAGADDLT

SEQ ID NO: 169 - protein, 9 aa, Artificial
  GG repeat
GGAGADNFI

SEQ ID NO: 170 - protein, 9 aa, Artificial
  GG repeat
GGAGNDEVH

SEQ ID NO: 171 - protein, 9 aa, Artificial
  GG repeat
GGAGNDYLS

SEQ ID NO: 172 - protein, 9 aa, Artificial
  GG repeat
GGAGNDSLF

SEQ ID NO: 173 - protein, 9 aa, Artificial
  GG repeat
GGAGFDILI

SEQ ID NO: 174 - protein, 9 aa, Artificial
  GG repeat
GGAGNDVAF

SEQ ID NO: 175 - protein, 10 aa, Artificial
  GG repeat
GGAGNNDIYH

SEQ ID NO: 176 - protein, 9 aa, Artificial
  GG repeat
GGAGADSFV

SEQ ID NO: 177 - protein, 9 aa, Artificial
  GG repeat
GGAGADALY

SEQ ID NO: 178 - protein, 9 aa, Artificial
  GG repeat
GGAGSDAIV

SEQ ID NO: 179 - protein, 9 aa, Artificial
  GG repeat
GGAGEDTFR

SEQ ID NO: 180 - protein, 9 aa, Artificial
  GG repeat
GGAGHDRLA

SEQ ID NO: 181 - protein, 9 aa, Artificial
  GG repeat
GGAGADTFV

SEQ ID NO: 182 - protein, 9 aa, Artificial
  GG repeat
GGAGDDQLS

SEQ ID NO: 183 - protein, 9 aa, Artificial
  GG repeat
GGAGDDVLE

SEQ ID NO: 184 - protein, 9 aa, Artificial
  GG repeat
GGAGTDHLD

SEQ ID NO: 185 - protein, 9 aa, Artificial
  GG repeat
GGAGNDRID

SEQ ID NO: 186 - protein, 9 aa, Artificial
  GG repeat
GGAGADQLW

SEQ ID NO: 187 - protein, 9 aa, Artificial
  GG repeat
GGAGNDTFV

SEQ ID NO: 188 - protein, 9 aa, Artificial
  GG repeat
GGAGGDLLD

SEQ ID NO: 189 - protein, 9 aa, Artificial
  GG repeat
GGAGEDSFR

SEQ ID NO: 190 - protein, 9 aa, Artificial
  GG repeat
GGAGNDLME

SEQ ID NO: 191 - protein, 9 aa, Artificial
  GG repeat
GGAGMDALH

SEQ ID NO: 192 - protein, 9 aa, Artificial
  GG repeat
GGAGTDTLV

SEQ ID NO: 193 - protein, 9 aa, Artificial
  GG repeat
GGAGADTLY

SEQ ID NO: 194 - protein, 9 aa, Artificial
  GG repeat
GGAGADELT

SEQ ID NO: 195 - protein, 9 aa, Artificial
  GG repeat
GGDGNDVLL

SEQ ID NO: 196 - protein, 9 aa, Artificial
  GG repeat
GGDGADLLF

SEQ ID NO: 197 - protein, 9 aa, Artificial
  GG repeat
GGDGTDFLL

SEQ ID NO: 198 - protein, 9 aa, Artificial
  GG repeat
GGEGHDFVS

SEQ ID NO: 199 - protein, 9 aa, Artificial
  GG repeat
GGEGDDRVY

SEQ ID NO: 200 - protein, 9 aa, Artificial
  GG repeat
GGEGADLLF

SEQ ID NO: 201 - protein, 9 aa, Artificial
  GG repeat
GGEGNDHLR

SEQ ID NO: 202 - protein, 9 aa, Artificial
  GG repeat
GGEGADRLI

SEQ ID NO: 203 - protein, 9 aa, Artificial
  GG repeat
GGFGNDEVN

SEQ ID NO: 204 - protein, 9 aa, Artificial
  GG repeat
GGGGSDIMR

SEQ ID NO: 205 - protein, 9 aa, Artificial
  GG repeat
GGGGNDILI

SEQ ID NO: 206 - protein, 9 aa, Artificial
  GG repeat
GGGGNDRLE

SEQ ID NO: 207 - protein, 9 aa, Artificial
  GG repeat
GGGGSDMFV

SEQ ID NO: 208 - protein, 9 aa, Artificial
  GG repeat
GGLGSDVLD

SEQ ID NO: 209 - protein, 9 aa, Artificial
  GG repeat
GGLGSDQLF

SEQ ID NO: 210 - protein, 9 aa, Artificial
  GG repeat
GGLGADTLI

SEQ ID NO: 211 - protein, 9 aa, Artificial
  GG repeat
GGMGADELT

SEQ ID NO: 212 - protein, 9 aa, Artificial
  GG repeat
GGQGRDQLH

SEQ ID NO: 213 - protein, 9 aa, Artificial
  GG repeat
GGRGSDIFA

SEQ ID NO: 214 - protein, 9 aa, Artificial
  GG repeat
GGRGSDLLD

SEQ ID NO: 215 - protein, 9 aa, Artificial
  GG repeat
GGSGNDRLI

SEQ ID NO: 216 - protein, 9 aa, Artificial
  GG repeat
GGSGNDRLD

SEQ ID NO: 217 - protein, 9 aa, Artificial
  GG repeat
GGSGNDDLS

SEQ ID NO: 218 - protein, 9 aa, Artificial
  GG repeat
GGSGDDRYQ

SEQ ID NO: 219 - protein, 9 aa, Artificial
  GG repeat
GGSGSDTFV

SEQ ID NO: 220 - protein, 9 aa, Artificial
  GG repeat
GGTGNDLVS

SEQ ID NO: 221 - protein, 9 aa, Artificial
  GG repeat
GGTGGDTLS

SEQ ID NO: 222 - protein, 9 aa, Artificial
  GG repeat
GGTGHDTLI

SEQ ID NO: 223 - protein, 9 aa, Artificial
  GG repeat
GGTGSDRLV

SEQ ID NO: 224 - protein, 9 aa, Artificial
  GG repeat
GGTGNDTYI

SEQ ID NO: 225 - protein, 9 aa, Artificial
  GG repeat
GGTGRDVFL

SEQ ID NO: 226 - protein, 9 aa, Artificial
  GG repeat
GGVGADTMT

SEQ ID NO: 227 - protein, 9 aa, Artificial
  GG repeat
GGEGRDSLY

SEQ ID NO: 228 - protein, 66 aa, Artificial
  HlyA fragment
GNSLAKNVLFGGKGNDKLYGSEGADLLDGGEGDDLLKGGYGNDIYRYLSGYGHHIIDDDGGKEDKL

SEQ ID NO: 229 - protein, 130 aa, Artificial; identical to SEQ ID NO: 86
  RTX12 Fragment P2111

SEQ ID NO: 230 - protein, 140 aa, Artificial; identical to SEQ ID NO: 81
  RTX12 Fragment P2106

SEQ ID NO: 231 - protein, 140 aa, Artificial; identical to SEQ ID NO: 83
  RTX12 Fragment P2108

SEQ ID NO: 232 - protein, 150 aa, Artificial; identical to SEQ ID NO: 84
  RTX12 Fragment P2109

SEQ ID NO: 233 - protein, 160 aa, Artificial; identical to SEQ ID NO: 85
  RTX12 Fragment P2110

SEQ ID NO: 234 - protein, 120 aa, Artificial; identical to SEQ ID NO: 82
  RTX12 Fragment P2107

SEQ ID NO: 235 - protein, 54 aa, Artificial; identical to SEQ ID NO: 87
  RTX12 GG repeat core

SEQ ID NO: 236 - protein, 150 aa, Artificial
  RTX18 Fragment P2141
VVGATNFAGTGNGVGNTLTGGTGGDTLSGLGGADVLNGGDGNDVLLGGAGADDLTGGVGADTMTGGAGADNFIYTNVDQSTAALRDLIIDFGATDDIDLGAIDANAGTVANNAFSFIGTAAFGANATGQLRYSFVGANTLVEASNDADAV

SEQ ID NO: 237 - protein, 140 aa, Artificial
  RTX18 Fragment P2140
VVGATNFAGTGNGVGNTLTGGTGGDTLSGLGGADVLNGGDGNDVLLGGAGADDLTGGVGADTMTGGAGADNFIYTNVDQSTAALRDLIIDFGATDDIDLGAIDANAGTVANNAFSFIGTAAFGANATGQLRYSFVGANTL

SEQ ID NO: 238 - protein, 140 aa, Artificial
  RTX18 Fragment P2142
VQVQSLLNAFTLAANFENLLVVGATNFAGTGNGVGNTLTGGTGGDTLSGLGGADVLNGGDGNDVLLGGAGADDLTGGVGADTMTGGAGADNFIYTNVDQSTAALRDLIIDFGATDDIDLGAIDANAGTVANNAFSFIGTA

SEQ ID NO: 239 - protein, 170 aa, Artificial
  RTX18 Fragment P2139
VQVQSLLNAFTLAANFENLLVVGATNFAGTGNGVGNTLTGGTGGDTLSGLGGADVLNGGDGNDVLLGGAGADDLTGGVGADTMTGGAGADNFIYTNVDQSTAALRDLIIDFGATDDIDLGAIDANAGTVANNAFSFIGTAAFGANATGQLRYSFVGANTLVEASNDADAV

SEQ ID NO: 240 - protein, 150 aa, Artificial
  RTX18 Fragment P2143
VQVQSLLNAFTLAANFENLLVVGATNFAGTGNGVGNTLTGGTGGDTLSGLGGADVLNGGDGNDVLLGGAGADDLTGGVGADTMTGGAGADNFIYTNVDQSTAALRDLIIDFGATDDIDLGAIDANAGTVANNAFSFIGTAAFGANATGQL

SEQ ID NO: 241 - protein, 54 aa, Artificial
  RTX18 GG repeat core
GGTGGDTLSGLGGADVLNGGDGNDVLLGGAGADDLTGGVGADTMTGGAGADNFI

SEQ ID NO: 242 - protein, 168 aa, Artificial
  RTX22 Fragment P2189
RVFGSAGSDLVFGEQGNDFVGAGEGNDFASGGAGNDEVHGESGDDYVFGDEGDDQLFGGEGRDSLYGGLGSDVLDGDAGDDLLAGEQGDDVLMGGAGNDYLSGGLGSDQLFGGDGADLLFGNAGNDSLSGGRGSDIFAYGRGDGQDVIRDFTVGGSERDVIAFNGGVF

SEQ ID NO: 243 - protein, 183 aa, Artificial
  RTX22 FRagment P2197
RVFGSAGSDLVFGEQGNDFVGAGEGNDFASGGAGNDEVHGESGDDYVFGDEGDDQLFGGEGRDSLYGGLGSDVLDGDAGDDLLAGEQGDDVLMGGAGNDYLSGGLGSDQLFGGDGADLLFGNAGNDSLSGGRGSDIFAYGRGDGQDVIRDFTVGGSERDVIAFNGGVFTSFAAVQAATQQLGA

SEQ ID NO: 244 - protein, 163 aa, Artificial
  RTX22 Fragment P2187
GAGEGNDFASGGAGNDEVHGESGDDYVFGDEGDDQLFGGEGRDSLYGGLGSDVLDGDAGDDLLAGEQGDDVLMGGAGNDYLSGGLGSDQLFGGDGADLLFGNAGNDSLSGGRGSDIFAYGRGDGQDVIRDFTVGGSERDVIAFNGGVFTSFAAVQAATQQLGA

SEQ ID NO: 245 - protein, 178 aa, Artificial
  RTX22 Fragment P2188
GAGEGNDFASGGAGNDEVHGESGDDYVFGDEGDDQLFGGEGRDSLYGGLGSDVLDGDAGDDLLAGEQGDDVLMGGAGNDYLSGGLGSDQLFGGDGADLLFGNAGNDSLSGGRGSDIFAYGRGDGQDVIRDFTVGGSERDVIAFNGGVFTSFAAVQAATQQLGADAVITVGAGDSLTLQ

SEQ ID NO: 246 - protein, 108 aa, Artificial
  RTX22 GG repeat core
GGAGNDEVHGESGDDYVFGDEGDDQLFGGEGRDSLYGGLGSDVLDGDAGDDLLAGEQGDDVLMGGAGNDYLSGGLGSDQLFGGDGADLLFGNAGNDSLSGGRGSDIFA

SEQ ID NO: 247 - protein, 162 aa, Artificial
  RTX30 Fragment P2244
RGGGAADRMIGGTGNDTYIVDHAGDEVVERAGEGLDRVCASVSHQLAAHVERLMLTGTAAINGAGNGLANVLWGNGAANRLSGGGGNDRLEGRDGSDVLNGGTGHDTLIGGAGHDRLAGGTGSDRLVGGSGNDRLDGGIDRDVLTGGAGADTFVFAAGYGRD

SEQ ID NO: 248 - protein, 187 aa, Artificial
  RTX30 Fragment P2241
LYGFGGNDTLRGGGAADRMIGGTGNDTYIVDHAGDEVVERAGEGLDRVCASVSHQLAAHVERLMLTGTAAINGAGNGLANVLWGNGAANRLSGGGGNDRLEGRDGSDVLNGGTGHDTLIGGAGHDRLAGGTGSDRLVGGSGNDRLDGGIDRDVLTGGAGADTFVFAAGYGRDVITDFRVGNGGDRLE

SEQ ID NO: 249 - protein, 172 aa, Artificial
  RTX30 Fragment P2242
LYGFGGNDTLRGGGAADRMIGGTGNDTYIVDHAGDEVVERAGEGLDRVCASVSHQLAAHVERLMLTGTAAINGAGNGLANVLWGNGAANRLSGGGGNDRLEGRDGSDVLNGGTGHDTLIGGAGHDRLAGGTGSDRLVGGSGNDRLDGGIDRDVLTGGAGADTFVFAAGYGRD

SEQ ID NO: 250 - protein, 202 aa, Artificial
  RTX30 Fragment P2243
LYGFGGNDTLRGGGAADRMIGGTGNDTYIVDHAGDEVVERAGEGLDRVCASVSHQLAAHVERLMLTGTAAINGAGNGLANVLWGNGAANRLSGGGGNDRLEGRDGSDVLNGGTGHDTLIGGAGHDRLAGGTGSDRLVGGSGNDRLDGGIDRDVLTGGAGADTFVFAAGYGRDVITDFRVGNGGDRLEIHDYAAPLELRQVGG

SEQ ID NO: 251 - protein, 72 aa, Artificial
  RTX30 GG repeat core
GGGGNDRLEGRDGSDVLNGGTGHDTLIGGAGHDRLAGGTGSDRLVGGSGNDRLDGGIDRDVLTGGAGADTFV

SEQ ID NO: 252 - protein, 144 aa, Artificial
  RTX30 GG repeat core
GGTGNDTYIVDHAGDEVVERAGEGLDRVCASVSHQLAAHVERLMLTGTAAINGAGNGLANVLWGNGAANRLSGGGGNDRLEGRDGSDVLNGGTGHDTLIGGAGHDRLAGGTGSDRLVGGSGNDRLDGGIDRDVLTGGAGADTFV

SEQ ID NO: 253 - protein, 187 aa, Artificial
  RTX33 Fragment P2256
YGGAKDDDMDGGAGDDQLSGEAGSDILHGGSGNDDLSGNAGDDLLKGGEGNDHLRGGQGRDQLHGEAGDDRLEGGAGDDVLEGGAGTDHLDGGSGDDRYQFGRGAGADTITDTAGSADELTIGANVATDQLWLRRVGQNLEVSIIGTADKCTINNWYAGSANRVERFSTSAGNALLNSQFDALVSAM

SEQ ID NO: 254 - protein, 157 aa, Artificial
  RTX33 Fragment P2257
YGGAKDDDMDGGAGDDQLSGEAGSDILHGGSGNDDLSGNAGDDLLKGGEGNDHLRGGQGRDQLHGEAGDDRLEGGAGDDVLEGGAGTDHLDGGSGDDRYQFGRGAGADTITDTAGSADELTIGANVATDQLWLRRVGQNLEVSIIGTADKCTINNWY

SEQ ID NO: 255 - protein, 172 aa, Artificial
  RTX33 Fragment P2255
YGGAKDDDMDGGAGDDQLSGEAGSDILHGGSGNDDLSGNAGDDLLKGGEGNDHLRGGQGRDQLHGEAGDDRLEGGAGDDVLEGGAGTDHLDGGSGDDRYQFGRGAGADTITDTAGSADELTIGANVATDQLWLRRVGQNLEVSIIGTADKCTINNWYAGSANRVERFSTSAG

SEQ ID NO: 256 - protein, 90 aa, Artificial
  RTX33 GG repeat core
GGAGDDQLSGEAGSDILHGGSGNDDLSGNAGDDLLKGGEGNDHLRGGQGRDQLHGEAGDDRLEGGAGDDVLEGGAGTDHLDGGSGDDRYQ

SEQ ID NO: 257 - protein, 151 aa, Artificial
  RTX28 Fragment P2233
STSSRDSSTVFTVEETGTYYIVEGSWSEDAPGDGWSDAVPEGSDYKLNVSVEFPPEPVRPGEAGRDRLVGGAGGDLLDGGLAADVLLGGAGEDSFRFSTALGAGNVDRILDFEVGEDLILLDRAIFAEAGGAGTLSFGAFHRSPSGAAQDA

SEQ ID NO: 258 - protein, 156 aa, Artificial
  RTX28 Fragment P2234
VLDSEGRIVAENDDGGGDPGSTSSRDSSTVFTVEETGTYYIVEGSWSEDAPGDGWSDAVPEGSDYKLNVSVEFPPEPVRPGEAGRDRLVGGAGGDLLDGGLAADVLLGGAGEDSFRFSTALGAGNVDRILDFEVGEDLILLDRAIFAEAGGAGTLS

SEQ ID NO: 259 - protein, 171 aa, Artificial
  RTX28 Fragment P2236
VLDSEGRIVAENDDGGGDPGSTSSRDSSTVFTVEETGTYYIVEGSWSEDAPGDGWSDAVPEGSDYKLNVSVEFPPEPVRPGEAGRDRLVGGAGGDLLDGGLAADVLLGGAGEDSFRFSTALGAGNVDRILDFEVGEDLILLDRAIFAEAGGAGTLSFGAFHRSPSGAAQDA

SEQ ID NO: 260 - protein, 186 aa, Artificial
  RTX28 Fragment P2235
VLDSEGRIVAENDDGGGDPGSTSSRDSSTVFTVEETGTYYIVEGSWSEDAPGDGWSDAVPEGSDYKLNVSVEFPPEPVRPGEAGRDRLVGGAGGDLLDGGLAADVLLGGAGEDSFRFSTALGAGNVDRILDFEVGEDLILLDRAIFAEAGGAGTLSFGAFHRSPSGAAQDASDRILYDTDSGFLSY

SEQ ID NO: 261 - protein, 27 aa, Artificial
  RTX28 GG repeat core
GGAGGDLLDGGLAADVLLGGAGEDSFR

SEQ ID NO: 262 - protein, 166 aa, Artificial
  RTX24 Fragment P2190
VKNYSFASDGAGTLYLRDANGGISMTRDIGAIQSKEPGFLWGLFKDDVIHQVTDQGLKAGGQLTQYASSVRGDAGDNVLKAHAGGDWLFGLDGNDHLIGGQGNDVFVGGAGNDLMEAGGGNNTFLFTGHFGQDRILGYQGGDKLVFMGVSGVLPDQDYRAHASSSG

SEQ ID NO: 263 - protein, 181 aa, Artificial
  RTX24 Fragment P2191
VKNYSFASDGAGTLYLRDANGGISMTRDIGAIQSKEPGFLWGLFKDDVIHQVTDQGLKAGGQLTQYASSVRGDAGDNVLKAHAGGDWLFGLDGNDHLIGGQGNDVFVGGAGNDLMEAGGGNNTFLFTGHFGQDRILGYQGGDKLVFMGVSGVLPDQDYRAHASSSGNDTVLTFGQDSVTLV

SEQ ID NO: 264 - protein, 136 aa, Artificial
  RTX24 Fragment P2192
VKNYSFASDGAGTLYLRDANGGISMTRDIGAIQSKEPGFLWGLFKDDVIHQVTDQGLKAGGQLTQYASSVRGDAGDNVLKAHAGGDWLFGLDGNDHLIGGQGNDVFVGGAGNDLMEAGGGNNTFLFTGHFGQDRIL

SEQ ID NO: 265 - protein, 151 aa, Artificial
  RTX24 Fragment P2193
VKNYSFASDGAGTLYLRDANGGISMTRDIGAIQSKEPGFLWGLFKDDVIHQVTDQGLKAGGQLTQYASSVRGDAGDNVLKAHAGGDWLFGLDGNDHLIGGQGNDVFVGGAGNDLMEAGGGNNTFLFTGHFGQDRILGYQGGDKLVFMGVSG

SEQ ID NO: 266 - protein, 186 aa, Artificial
  RTX24 Fragment P2194
GGYNIILGGQGSNTLDLQQSVKNYSFASDGAGTLYLRDANGGISMTRDIGAIQSKEPGFLWGLFKDDVIHQVTDQGLKAGGQLTQYASSVRGDAGDNVLKAHAGGDWLFGLDGNDHLIGGQGNDVFVGGAGNDLMEAGGGNNTFLFTGHFGQDRILGYQGGDKLVFMGVSGVLPDQDYRAHASSSG

SEQ ID NO: 267 - protein, 156 aa, Artificial
  RTX24 Fragment P2195
GGYNIILGGQGSNTLDLQQSVKNYSFASDGAGTLYLRDANGGISMTRDIGAIQSKEPGFLWGLFKDDVIHQVTDQGLKAGGQLTQYASSVRGDAGDNVLKAHAGGDWLFGLDGNDHLIGGQGNDVFVGGAGNDLMEAGGGNNTFLFTGHFGQDRIL

SEQ ID NO: 268 - protein, 171 aa, Artificial
  RTX24 Fragment P2196
GGYNIILGGQGSNTLDLQQSVKNYSFASDGAGTLYLRDANGGISMTRDIGAIQSKEPGFLWGLFKDDVIHQVTDQGLKAGGQLTQYASSVRGDAGDNVLKAHAGGDWLFGLDGNDHLIGGQGNDVFVGGAGNDLMEAGGGNNTFLFTGHFGQDRILGYQGGDKLVFMGVSG

SEQ ID NO: 269 - protein, 18 aa, Artificial
  RTX24 GG repeat core
GGQGNDVFVGGAGNDLME

SEQ ID NO: 270 - protein, 160 aa, Artificial
  RTX19 Fragment P2145
VFVTPEFTGSILGNADLDGTALYGSTLNDLLSSGAGDQTLFGDAGDDFLEGGAGMDALHGEDGNDFLVWDDTDSTIDGGAGTDTLVLFEEDYLLNLSGLSGTVLTDIEAIDITGDGAVTMQLTVQDVLDLSSTTDQLLIHGNSDDVVQTAAASWAQGADQ

SEQ ID NO: 271 - protein, 130 aa, Artificial
  RTX19 Fragment P2144
ALYGSTLNDLLSSGAGDQTLFGDAGDDFLEGGAGMDALHGEDGNDFLVWDDTDSTIDGGAGTDTLVLFEEDYLLNLSGLSGTVLTDIEAIDITGDGAVTMQLTVQDVLDLSSTTDQLLIHGNSDDVVQTA

SEQ ID NO: 272 - protein, 140 aa, Artificial
  RTX19 Fragment P2146
VFVTPEFTGSILGNADLDGTALYGSTLNDLLSSGAGDQTLFGDAGDDFLEGGAGMDALHGEDGNDFLVWDDTDSTIDGGAGTDTLVLFEEDYLLNLSGLSGTVLTDIEAIDITGDGAVTMQLTVQDVLDLSSTTDQLLIH

SEQ ID NO: 273 - protein, 120 aa, Artificial
  RTX19 Fragment P2147
ALYGSTLNDLLSSGAGDQTLFGDAGDDFLEGGAGMDALHGEDGNDFLVWDDTDSTIDGGAGTDTLVLFEEDYLLNLSGLSGTVLTDIEAIDITGDGAVTMQLTVQDVLDLSSTTDQLLIH

SEQ ID NO: 274 - protein, 36 aa, Artificial
  RTX19 GG repeat core
GGAGMDALHGEDGNDFLVWDDTDSTIDGGAGTDTLV

SEQ ID NO: 275 - protein, 160 aa, Artificial
  RTX16 Fragment P2123
TENADEGIDLVQSSTGWTLGANVEHLELTGTANTRGTGNDLDNVITGNSGKNYLAGEEGEDILLGGAGADTLYGGMGADELTGGTGRDVFLYNNSSESGITEGAWDVITDFTAGFDRLDLSGIDANTGIAGNQSFSSDILGSGDAFSEAGQLRFDSGSGV

SEQ ID NO: 276 - protein, 130 aa, Artificial
  RTX16 Fragment P2124
ANVEHLELTGTANTRGTGNDLDNVITGNSGKNYLAGEEGEDILLGGAGADTLYGGMGADELTGGTGRDVFLYNNSSESGITEGAWDVITDFTAGFDRLDLSGIDANTGIAGNQSFSSDILGSGDAFSEAG

SEQ ID NO: 277 - protein, 140 aa, Artificial
  RTX19 Fragment P2138
TENADEGIDLVQSSTGWTLGANVEHLELTGTANTRGTGNDLDNVITGNSGKNYLAGEEGEDILLGGAGADTLYGGMGADELTGGTGRDVFLYNNSSESGITEGAWDVITDFTAGFDRLDLSGIDANTGIAGNQSFSSDIL

SEQ ID NO: 278 - protein, 27 aa, Artificial
  RTX16 GG repeat core
GGAGADTLYGGMGADELTGGTGRDVFL

SEQ ID NO: 279 - protein, 171 aa, Artificial
  RTX26 Fragment P2230
NYSTRAGDTTYGFNSNTGRDFYSASSSSDKLVFSVWDGGGNDTLDFSGFTQNQKINLNEASFSDVGGMVGNVSIAQGVTVENAIGGSGNDLLIGNNAANVLKGGAGNDIIYGAGGADQLWGGSGSDTFVFAASTDSKPGAADQIMDFVSGLDKIDLTGITKGAGLHFVNAF

SEQ ID NO: 280 - protein, 136 aa, Artificial
  RTX26 Fragment P2231
FYSASSSSDKLVFSVWDGGGNDTLDFSGFTQNQKINLNEASFSDVGGMVGNVSIAQGVTVENAIGGSGNDLLIGNNAANVLKGGAGNDIIYGAGGADQLWGGSGSDTFVFAASTDSKPGAADQIMDFVSGLDKIDL

SEQ ID NO: 281 - protein, 186 aa, Artificial
  RTX26 Fragment P2232
NYSTRAGDTTYGFNSNTGRDFYSASSSSDKLVFSVWDGGGNDTLDFSGFTQNQKINLNEASFSDVGGMVGNVSIAQGVTVENAIGGSGNDLLIGNNAANVLKGGAGNDIIYGAGGADQLWGGSGSDTFVFAASTDSKPGAADQIMDFVSGLDKIDLTGITKGAGLHFVNAFTGAAGDAILTTSGGV

SEQ ID NO: 282 - protein, 45 aa, Artificial
  RTX26 GG repeat core
GGSGNDLLIGNNAANVLKGGAGNDIIYGAGGADQLWGGSGSDTFV

SEQ ID NO: 283 - protein, 39 aa, Artificial; identical to SEQ ID NO: 53
  HlyA1 Fragment P2102

SEQ ID NO: 284 - protein, 44 aa, Artificial; identical to SEQ ID NO: 52
  HlyA1 Fragment P2101

SEQ ID NO: 285 - protein, 49 aa, Artificial; identical to SEQ ID NO: 51
  HlyA1 Fragment P2100

SEQ ID NO: 286 - protein, 54 aa, Artificial; identical to SEQ ID NO: 50
  HlyA1 Fragment P2099

SEQ ID NO: 287 - protein, 59 aa, Artificial; identical to SEQ ID NO: 49
  HlyA1 Fragment P2098

SEQ ID NO: 288 - protein, 160 aa, Artificial
  RTX3 Fragment P2070
NDISNKLSGEAGNDTLSGFGGVDYLEGGAGNDSLFGGDGTDFLLGGAGADRLNGGAGFDILIGGAGNDVYAFDVSAFPDSDGLSIDFVSDFERGDLFDFSALDANATLEGDQAFSLVSQWAANGVGQVMVSTYSNVVKAVGALGGVDLGALGWGLGGGVS

SEQ ID NO: 289 - protein, 45 aa, Artificial
  RTX3 GG repeat core
GGAGNDSLFGGDGTDFLLGGAGADRLNGGAGFDILIGGAGNDVYA

SEQ ID NO: 290 - protein, 150 aa, Artificial
  RTX20 Fragment P2165
LTGTSGTDAIHGNIQSEIIDGGAGADALYGGGGNDILIYDDLDYYVDGGAGSDAIVVSGAGITIGGATTQTGHGDVHNIEAVDLTGTGDNTLYLDLNQVLDITDSKQTLLVYGNSGDTLYMNDFNSWNLATTGSMTNPTSSGNTINYNLY

SEQ ID NO: 291 - protein, 36 aa, Artificial
  RTX20 GG repeat core
GGAGADALYGGGGNDILIYDDLDYYVDGGAGSDAIV

SEQ ID NO: 292 - protein, 156 aa, Artificial
  RTX31 Fragment P2250
LLDSAGNIVAQNDDGGGDPGSTSNRDSSLVFVAQETGTYYILEGSWSPTAPGDGWAEAVPAGSTYELNVSVEFPPAPAQPGVSGSDTLIGGRGSDLLDGGLGADTLIGGAGEDTFRFSTALGNGNVDGIREFDVIDDTILLDNLIFESVGGDGALA

SEQ ID NO: 293 - protein, 27 aa, Artificial
  RTX31 GG repeat core
GGRGSDLLDGGLGADTLIGGAGEDTFR

SEQ ID NO: 294 - protein, 171 aa, Artificial
  RTX14 Fragment P2121
ATGNELDNRIMGNAGDNRIDGGAGNDLMIGGAGNDTYVIDSIRDLVTEKVGEGYDTVESKVTWTLGANVEKLVLIGTAAIDGAGNADSNTLIGNSAANRLDGGAGNDRIDGGAGADQLWGGAGNDTFVYTSTHFGKDVIKDFGTGFDVLEFSQAPYGDYASVMSHTQQVGV

SEQ ID NO: 295 - protein, 108 aa, Artificial
  RTX14 GG repeat core
GGAGNDLMIGGAGNDTYVIDSIRDLVTEKVGEGYDTVESKVTWTLGANVEKLVLIGTAAIDGAGNADSNTLIGNSAANRLDGGAGNDRIDGGAGADQLWGGAGNDTFV

SEQ ID NO: 296 - protein, 183 aa, Artificial
  RTX32 Fragment P2252
LNGLAGNDVLHGRAGNDTLIGGDGNDSLVGGAGADRLDGGNGVDLANYYNAALGLVADLYSPVSNTGEAAGDTYLSVERLYGSAFNDSLRGDNIANLLNGLAGNDVLNGRGGNDTLIGGEGADRLIGGGGSDMFVFQTATQSRPAAMDVIDDFARGIDRIDLRSIDASSNLSGDQAFLFIGGN

SEQ ID NO: 297 - protein, 27 aa, Artificial
  RTX32 GG repeat core
GGDGNDSLVGGAGADRLDGGNGVDLAN

SEQ ID NO: 298 - protein, 115 aa, Artificial
  RTX32 GG repeat core
GGDGNDSLVGGAGADRLDGGNGVDLANYYNAALGLVADLYSPVSNTGEAAGDTYLSVERLYGSAFNDSLRGDNIANLLNGLAGNDVLNGRGGNDTLIGGEGADRLIGGGGSDMFV

SEQ ID NO: 299 - protein, 156 aa, Artificial
  RTX29 Fragment P2239
APGTVIEAARGGSGNDRLIGNGVANVLNGGGGSDIMRGGAGNDIYHSDGLDRIVELPGQGIDRVISTASCTLAANVENLTLAGGRSISGTGNGLANVIAGNAAANRLTGGAGADSFVFATALGRGNVDRITDFSVADDTIRLDDAIFRALPRGSLA

SEQ ID NO: 300 - protein, 107 aa, Artificial
  RTX29 GG repeat core
GGSGNDRLIGNGVANVLNGGGGSDIMRGGAGNDIYHSDGLDRIVELPGQGIDRVISTASCTLAANVENLTLAGGRSISGTGNGLANVIAGNAAANRLTGGAGADSFV

SEQ ID NO: 301 - protein, 9 aa, Artificial
  GG repeat-like motif
AGEGNDFAS

SEQ ID NO: 302 - protein, 9 aa, Artificial
  GG repeat-like motif
AGYGRDVIT

SEQ ID NO: 303 - protein, 9 aa, Artificial
  GG repeat-like motif
GAAGDDQVY

SEQ ID NO: 304 - protein, 9 aa, Artificial
  GG repeat-like motif
GAGGADQLW

SEQ ID NO: 305 - protein, 9 aa, Artificial
  GG repeat-like motif
GDAGNDLLF

SEQ ID NO: 306 - protein, 9 aa, Artificial
  GG repeat-like motif
GDDGDDAVS

SEQ ID NO: 307 - protein, 9 aa, Artificial
  GG repeat-like motif
GDAGDDFLE

SEQ ID NO: 308 - protein, 9 aa, Artificial
  GG repeat-like motif
GDAGDDLLA

SEQ ID NO: 309 - protein, 9 aa, Artificial
  GG repeat-like motif
GDEGDDQLF

SEQ ID NO: 310 - protein, 9 aa, Artificial
  GG repeat-like motif
GEAGADQVF

SEQ ID NO: 311 - protein, 9 aa, Artificial
  GG repeat-like motif
GEAGDDRLE

SEQ ID NO: 312 - protein, 9 aa, Artificial
  GG repeat-like motif
GEAGNDTLS

SEQ ID NO: 313 - protein, 9 aa, Artificial
  GG repeat-like motif
GEAGRDRLV

SEQ ID NO: 314 - protein, 9 aa, Artificial
  GG repeat-like motif
GEAGSDILH

SEQ ID NO: 315 - protein, 9 aa, Artificial
  GG repeat-like motif
GEDGNDFLV

SEQ ID NO: 316 - protein, 9 aa, Artificial
  GG repeat-like motif
GEEGEDILL

SEQ ID NO: 317 - protein, 9 aa, Artificial
  GG repeat-like motif
GEQGDDVLM

SEQ ID NO: 318 - protein, 9 aa, Artificial
  GG repeat-like motif
GEQGEDVLL

SEQ ID NO: 319 - protein, 9 aa, Artificial
  GG repeat-like motif
GEQGNDFVG

SEQ ID NO: 320 - protein, 9 aa, Artificial
  GG repeat-like motif
GESGDDYVF

SEQ ID NO: 321 - protein, 9 aa, Artificial
  GG repeat-like motif
GEWGNDFVS

SEQ ID NO: 322 - protein, 9 aa, Artificial
  GG repeat-like motif
GFGGVDYLE

SEQ ID NO: 323 - protein, 9 aa, Artificial
  GG repeat-like motif
GGGAADRMI

SEQ ID NO: 324 - protein, 9 aa, Artificial
  GG repeat-like motif
GGIDRDVLT

SEQ ID NO: 325 - protein, 8 aa, Artificial
  GG repeat-like motif
GGKEDKLS

SEQ ID NO: 326 - protein, 9 aa, Artificial
  GG repeat-like motif
GGLAADVLL

SEQ ID NO: 327 - protein, 9 aa, Artificial
  GG repeat-like motif
GGLGSDAFA

SEQ ID NO: 328 - protein, 9 aa, Artificial
  GG repeat-like motif
GLAGNDVLH

SEQ ID NO: 329 - protein, 9 aa, Artificial
  GG repeat-like motif
GLAGNDVLN

SEQ ID NO: 330 - protein, 9 aa, Artificial
  GG repeat-like motif
GLDGNDHLI

SEQ ID NO: 331 - protein, 9 aa, Artificial
  GG repeat-like motif
GLGGADVLN

SEQ ID NO: 332 - protein, 9 aa, Artificial
  GG repeat-like motif
GNAGADALT

SEQ ID NO: 333 - protein, 9 aa, Artificial
  GG repeat-like motif
GNAGDDLLK

SEQ ID NO: 334 - protein, 9 aa, Artificial
  GG repeat-like motif
GNAGNDSLS

SEQ ID NO: 335 - protein, 9 aa, Artificial
  GG repeat-like motif
GRAGNDTLI

SEQ ID NO: 336 - protein, 9 aa, Artificial
  GG repeat-like motif
GRDGSDVLN

SEQ ID NO: 337 - protein, 9 aa, Artificial
  GG repeat-like motif
GRGGNDTLI

SEQ ID NO: 338 - protein, 9 aa, Artificial
  GG repeat-like motif
GSEGADLLD

SEQ ID NO: 339 - protein, 9 aa, Artificial
  GG repeat-like motif
GTSGTDAIH

SEQ ID NO: 340 - protein, 9 aa, Artificial
  GG repeat-like motif
GVSGSDTLI

SEQ ID NO: 341 - protein, 9 aa, Artificial
  GG repeat-like motif
RGAGADTIT

SEQ ID NO: 342 - protein, 170 aa, Artificial
  RTX12 Fragment 883-1052
IAATTKLGGGADEADYHTSLAAVKVDGGLGDDHLVGGRGSDLLIGGDGADRISGGAGNDIIRGGGGHDRMQGGAGDDTFVIGVGDLVTGDNQSDHIIDFRGAGSASGGEQDLLIFTGFSAGSKLVLDHDMGNTPTLQLYQVYNGASLVGSLLIQTNTAAHLTSGDYLFQA

SEQ ID NO: 343 - protein, 216 aa, Artificial
  RTX26 Fragment 267-482
NYSTRAGDTTYGFNSNTGRDFYSASSSSDKLVFSVWDGGGNDTLDFSGFTQNQKINLNEASFSDVGGMVGNVSIAQGVTVENAIGGSGNDLLIGNNAANVLKGGAGNDIIYGAGGADQLWGGSGSDTFVFAASTDSKPGAADQIMDFVSGLDKIDLTGITKGAGLHFVNAFTGAAGDAILTTSGGVSTLSVDFSGHGVADFLVSTVGQAAVSDIVA

SEQ ID NO: 344 - protein, 67 aa, Artificial
  Peptide of Interest
MCCNRGKNVSIENLHQGFTHIFESTFESTEGVAEYVSHPSHVEYANLFLANLEKVLVIDYKPTTVRV

SEQ ID NO: 345 - protein, 265 aa, Artificial
  Peptide of Interest (antibody fragment)
SGDASDSAEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARPTINSIAPQLWGQGTLVTVSSGGGGSGGGGSGGSALQSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLASVVFGGGTKLTVLGSGSTGENSSGTG

SEQ ID NO: 346 - protein, 160 aa, Artificial
  RTX9 Fragment P2103
EGLDRVCASVSHQLAAHVERLVLTGTAAINGAGNGLANVLWGNGAANRLSGGGGNDRLEGRDGSDVLNGGTGHDTLIGGAGHDRLAGGTGSDRLVGGSGNDRLDGGIDRDVLTGGAGADTFVFAAGYGREVITDFRVGNGGDRLEIHDYAAPLELRQVGG

SEQ ID NO: 347 - protein, 150 aa, Artificial
  RTX9 Fragment P2104
EGLDRVCASVSHQLAAHVERLVLTGTAAINGAGNGLANVLWGNGAANRLSGGGGNDRLEGRDGSDVLNGGTGHDTLIGGAGHDRLAGGTGSDRLVGGSGNDRLDGGIDRDVLTGGAGADTFVFAAGYGREVITDFRVGNGGDRLEIHDYA

SEQ ID NO: 348 - protein, 140 aa, Artificial
  RTX9 Fragment P2105
EGLDRVCASVSHQLAAHVERLVLTGTAAINGAGNGLANVLWGNGAANRLSGGGGNDRLEGRDGSDVLNGGTGHDTLIGGAGHDRLAGGTGSDRLVGGSGNDRLDGGIDRDVLTGGAGADTFVFAAGYGREVITDFRVGNG

SEQ ID NO: 349 - protein, 153 aa, Artificial
  RTX21 Fragment P2171
LVFGAAGDDQVYGEWGNDFVSGGEGHDFVSGGFGNDEVNGDAGNDLLFGDDGDDAVSGGEGDDRVYGGTGNDLVSGEAGADQVFGEQGEDVLLGGAGSDYLSGGAGADQLFGGEGADLLFGNAGADALTGGLGSDAFAFGRGDGQDVIRDFVT

SEQ ID NO: 350 - protein, 171 aa, Artificial
  RTX21 Fragment P2172
YGEWGNDFVSGGEGHDFVSGGFGNDEVNGDAGNDLLFGDDGDDAVSGGEGDDRVYGGTGNDLVSGEAGADQVFGEQGEDVLLGGAGSDYLSGGAGADQLFGGEGADLLFGNAGADALTGGLGSDAFAFGRGDGQDVIRDFVTGGAERDVIAFNNGAFGSFAVQAALRQEGS

SEQ ID NO: 351 - protein, 168 aa, Artificial
  RTX21 Fragment P2170
LVFGAAGDDQVYGEWGNDFVSGGEGHDFVSGGFGNDEVNGDAGNDLLFGDDGDDAVSGGEGDDRVYGGTGNDLVSGEAGADQVFGEQGEDVLLGGAGSDYLSGGAGADQLFGGEGADLLFGNAGADALTGGLGSDAFAFGRGDGQDVIRDFVTGGAERDVIAFNNGAF

SEQ ID NO: 352 - protein, 17 aa, Artificial
  HlyA sequence
GYGHHIIDDDGGKEDKL

SEQ ID NO: 353 - protein, 7 aa, Artificial
  HlyA fragment
GGKEDKL

SEQ ID NO: 354 - protein, 12 aa, Artificial
  HlyA fragment
IIDDDGGKEDKL

SEQ ID NO: 355 - protein, 20 aa, Artificial
  HlyA fragment
YLSGYGHHIIDDDGGKEDKL

SEQ ID NO: 356 - protein, 22 aa, Artificial
  HlyA fragment
YRYLSGYGHHIIDDDGGKEDKL

SEQ ID NO: 357 - protein, 27 aa, Artificial
  HlyA fragment
YGNDIYRYLSGYGHHIIDDDGGKEDKL

SEQ ID NO: 358 - protein, 9 aa, Artificial
  RTX21 GG repeat
GGLGSDAFA

SEQ ID NO: 359 - protein, 99 aa, Artificial
  RTX21 GG repeat core
GGEGHDFVSGGFGNDEVNGDAGNDLLFGDDGDDAVSGGEGDDRVYGGTGNDLVSGEAGADQVFGEQGEDVLLGGAGSDYLSGGAGADQLFGGEGADLLF

SEQ ID NO: 360 - protein, 72 aa, Artificial; identical to SEQ ID NO: 251
  RTX9 GG repeat core

SEQ ID NO: 361 - protein, 21 aa, Artificial
  Peptide of Interest SeqA
RFGRFLRKIRRFRPKVTITIQ

SEQ ID NO: 362 - protein, 18 aa, Artificial
  Peptide of Interest SeqB
KWCFRVCYRGICYRRCRD

SEQ ID NO: 363 - protein, 188 aa, Artificial
  Artificial GG repeat sequence
IAATTKLGGGADEADYHTSLAAVKVDGGLGDDHLVGGRGSDLLIGGDGADRISGGAGNDIIRGGGGHDRMQGGAGDDTFVGGLGDDHLVGGRGSDLLIGGDGADRISGGAGNDIIRGGGGHDRMQGGAGDDTFVGGLGDDHLVGGRGSDLLIGGDGADRISGGAGNDIIRGGGGHDRMQGGAGDDTFV

SEQ ID NO: 364 - protein, 27 aa, Artificial
  RTX1 GG repeat core
GGTGNDRLWGGAGADVLSGGTGADIFV

SEQ ID NO: 365 - protein, 186 aa, Artificial
  RTX31 fragment
LLDSAGNIVAQNDDGGGDPGSTSNRDSSLVFVAQETGTYYILEGSWSPTAPGDGWAEAVPAGSTYELNVSVEFPPAPAQPGVSGSDTLIGGRGSDLLDGGLGADTLIGGAGEDTFRFSTALGNGNVDGIREFDVIDDTILLDNLIFESVGGDGALAFGAFFGSAGGLAHDADDRIIYDTDSGVLSY

SEQ ID NO: 366 - protein, 168 aa, Artificial
  RTX32 fragment
LNGLAGNDVLHGRAGNDTLIGGDGNDSLVGGAGADRLDGGNGVDLANYYNAALGLVADLYSPVSNTGEAAGDTYLSVERLYGSAFNDSLRGDNIANLLNGLAGNDVLNGRGGNDTLIGGEGADRLIGGGGSDMFVFQTATQSRPAAMDVIDDFARGIDRIDLRSIDAS

SEQ ID NO: 367 - protein, 9 aa, Artificial
  GG repeat
GGDGNDSLV

SEQ ID NO: 368 - protein, 9 aa, Artificial
  GG repeat
GGAGADRLD

SEQ ID NO: 369 - protein, 9 aa, Artificial
  GG repeat
GGNGVDLAN

SEQ ID NO: 370 - protein, 130 aa, Artificial
  RTX1 fragment
APTLQPATITGTSGANTLKGTTGSDVILGLGGADKLYGRAGADQLDGGTGNDRLWGGAGADVLSGGTGADIFVFDAPFVGAVDRITDFSPKDDVIYLQNSIFTGLRAAGAMSNAAFHAGTKAHDATDRVI

SEQ ID NO: 371 - protein, 142 aa, Artificial
  RTX7 fragment
VAGADYSQFNLNGELDLYDQATGKGLSESWISDRAQMLSWMIQANTQDISTLPAITGDGLATIYSDWTSGKEVILRNDPIQSQHKVIFGRDSDQRDLIMGDNFSDRLYGGGGDDIIVGGKGDDYLEGGAGNDSYFINTGDGT